(19) United States
(12) Patent Application Publication
(10) Pub. No.: US 2011/0091808 A1
(43) Pub. Date: Apr. 21, 2011

(54) PHOTORESIST COMPOSITION

(75) Inventors: Tatsuro MASUYAMA, Toyonaka-shi (JP); Takashi HIRAOKA, Hannan-shi (JP)

(73) Assignee: SUMITOMO CHEMICAL COMPANY, LIMITED, Tokyo (JP)

(21) Appl. No.: 12/904,765

(22) Filed: Oct. 14, 2010

(30) Foreign Application Priority Data
Oct. 20, 2009 (JP) 2009-241206

(51) Int. Cl. G03F 7/004; C07C 309/17; G03F 7/20

(52) U.S. Cl. 430/270.1; 560/149; 430/325

(57) ABSTRACT

The present invention provides a photoresist composition comprising a compound capable of generating an acid and a base by irradiation, a resin having an acid-labile group and being insoluble or poorly soluble in an aqueous alkali solution but becoming soluble in an aqueous alkali solution by the action of an acid, and an acid generator.

This nonprovisional application claims priority under 35 U.S.C. §119(a) on Patent Application No. 2009-241206 filed in JAPAN on Oct. 20, 2009, the entire contents of which are hereby incorporated by reference.

### FIELD OF THE INVENTION

The present invention relates to a photoresist composition.

### BACKGROUND OF THE INVENTION

A photoresist composition is used for semiconductor microfabrication employing a lithography process.

US 2008/0166660 A1 discloses a photoresist composition comprising a resin having a structural unit derived from 2-ethyl-2-adamantyl methacrylate, a structural unit derived from 3-hydroxy-1-adamantyl methacrylate, a structural unit derived from 2-(5-oxo-4-oxatricyclo[4.2.1.0^{3,7}]nonan-2-yloxy)-2-oxoethyl methacrylate and a structural unit derived from α-methacryloyloxy-γ-butyrolactone, an acid generator comprising triphenylsulfonium 4-oxoadamantan-1-yloxycarbonyl(difluoro)methanesulfonate, a basic compound comprising 2,6-diisopropylaniline and solvents.

### SUMMARY OF THE INVENTION

The present invention is to provide a photoresist composition.

The present invention relates to the followings:

<1> A photoresist composition comprising a compound capable of generating an acid and a base by irradiation, a resin having an acid-labile group and being insoluble or poorly soluble in an aqueous alkali solution but becoming soluble in an aqueous alkali solution by the action of an acid, and an acid generator;
<2> The photoresist composition according to <1>, wherein the base generated by irradiation to the compound capable of generating an acid and a base by irradiation is a base represented by the formula (IB):

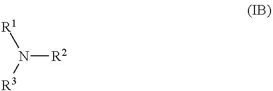

wherein R¹, R² and R³ each independently represent C1-C12 hydrocarbon group which can have one or more substituents, and two or three selected from the group consisting of R¹, R² and R³ can be bonded each other to form a ring together with the nitrogen atom to which they are bonded;
<3> The photoresist composition according to <1> or <2>, wherein the acid generated by irradiation to the compound capable of generating an acid and a base by irradiation is an acid represented by the formula (IA):

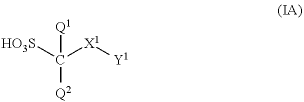

wherein Q¹ and Q² each independently represent a fluorine atom or a C1-C6 perfluoroalkyl group, X¹ represents a single bond or a C1-C17 divalent saturated hydrocarbon group in which one or more —CH₂— can be replaced by —O— or —CO—,
Y¹ represents a C1-C36 aliphatic hydrocarbon group which can have one or more substituents, a C3-C36 saturated cyclic hydrocarbon group which can have one or more substituents, or a C6-C36 aromatic hydrocarbon group which can have one or more substituents, and one or more —CH₂— in the aliphatic hydrocarbon group, the saturated cyclic hydrocarbon group and the aromatic hydrocarbon group can be replaced by —O— or —CO—;
<4> The photoresist composition according to <1>, wherein the compound capable of generating an acid and a base by irradiation is a salt represented by the formula (ID):

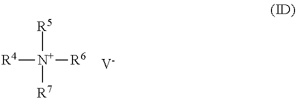

wherein R⁴, R⁵ and R⁶ each independently represent C1-C12 hydrocarbon group which can have one or more substituents, and two or three selected from the group consisting of R⁴, R⁵ and R⁶ can be bonded each other to form a ring together with the nitrogen atom to which they are bonded, R⁷ represents an organic group having an aromatic hydrocarbon group, and V⁻ represents an organic counter anion;
<5> The photoresist composition according to <4>, wherein R⁷ is a benzyl group which can have one or more substituents or a phenylethyl group which can have one or more substituents;
<6> A salt represented by the formula (I-1):

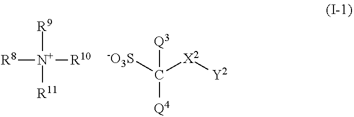

wherein R⁸, R⁹ and R¹⁰ each independently represent C1-C12 hydrocarbon group which can have one or more substituents, and two or three selected from the group consisting of R⁸, R⁹ and R¹⁰ can be bonded each other to form a ring together with the nitrogen atom to which they are bonded, R¹¹ represents a benzyl group which can have one or more substituents or a phenylethyl group which can have one or more substituents, Q³ and Q⁴ each independently represent a fluorine atom or a C1-C6 perfluoroalkyl group, X² represents a single bond or a C1-C17 divalent saturated hydrocarbon group in which one or more —CH₂— can be replaced by —O— or —CO—,
Y² represents a C1-C36 aliphatic hydrocarbon group which can have one or more substituents, a C3-C36 saturated cyclic hydrocarbon group which can have one or more substituents, or a C6-C36 aromatic hydrocarbon group which can have one or more substituents, and one or more —CH₂— in the aliphatic hydrocarbon group, the saturated cyclic hydrocarbon group and the aromatic hydrocarbon group can be replaced by —O— or —CO—;
<7> A process for producing a photoresist pattern comprising the following steps (1) to (5):

(1) a step of applying the photoresist composition according to any one of <1> to <6> on a substrate,

(2) a step of forming a photoresist film by conducting drying,

(3) a step of exposing the photoresist film to radiation,

(4) a step of baking the exposed photoresist film, and

(5) a step of developing the baked photoresist film with an alkaline developer, thereby forming a photoresist pattern.

### DESCRIPTION OF PREFERRED EMBODIMENTS

The photoresist composition of the present invention comprises the following three components:

a compound capable of generating an acid and a base by irradiation (hereinafter, simply referred to as Compound (D))

a resin having an acid-labile group and being insoluble or poorly soluble in an aqueous alkali solution but becoming soluble in an aqueous alkali solution by the action of an acid, and

an acid generator.

First, Compound (D) will be illustrated.

Compound (D) generates an acid and a base by irradiation of radiation such as ultraviolet rays (UV), KrF excimer laser, ArF excimer laser, extreme ultraviolet rays (EUV) and electron beam (EB).

Examples of the base generated from Compound (D) by irradiation include a base represented by the formula (IB):

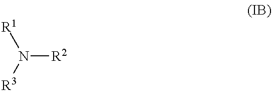

wherein R¹, R² and R³ each independently represent C1-C12 hydrocarbon group which can have one or more substituents, and two or three selected from the group consisting of R¹, R² and R³ can be bonded each other to form a ring together with the nitrogen atom to which they are bonded.

Examples of the C1-C12 hydrocarbon group include a C1-C12 aliphatic hydrocarbon group, a C3-C12 saturated cyclic hydrocarbon group and a C6-C12 aromatic hydrocarbon group. The aliphatic hydrocarbon group may be a linear aliphatic hydrocarbon group and may be a branched chain aliphatic hydrocarbon group. Examples of the linear aliphatic hydrocarbon group include a methyl group, an ethyl group, a propyl group, a butyl group, a pentyl group, a hexyl group, a heptyl group, an octyl group, a nonyl group, a decyl group, an undecyl group and a dodecyl group. Examples of the branched chain aliphatic hydrocarbon group include an isopropyl group, a sec-butyl group, a tert-butyl group, a methylpentyl group, an ethylpentyl group, a methylhexyl group, an ethylhexyl group, a propylhexyl group and a tert-octyl group, and an isopropyl group, a sec-butyl group, a tert-butyl group and an ethylhexyl group are preferable.

Examples of the C3-C12 saturated cyclic hydrocarbon group include the following groups.

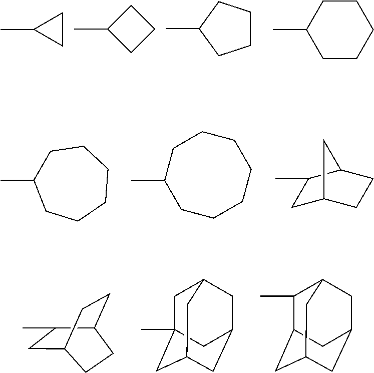

Examples of the C6-C12 aromatic hydrocarbon group include a phenyl group, a biphenyl group, a fluorenyl group, a naphthyl group and an anthryl group.

The C1-C12 hydrocarbon group can have one or more substituents. Examples of the substituent include a halogen atom such as a fluorine atom and a bromine atom, a nitro group, a cyano group, a hydroxyl group, and a C1-C6 hydroxyalkyl group such as a hydroxylmethyl group. The C1-C12 hydrocarbon group can have one or more connecting groups such as —SO₂—, —CO—, —O—CO— and —CO—O—.

Examples of the substituted C1-C12 hydrocarbon group include a halogenated aliphatic hydrocarbon group such as a trifluoromethyl group, a pentafluoroethyl group, a heptafluoropropyl group, and the followings.

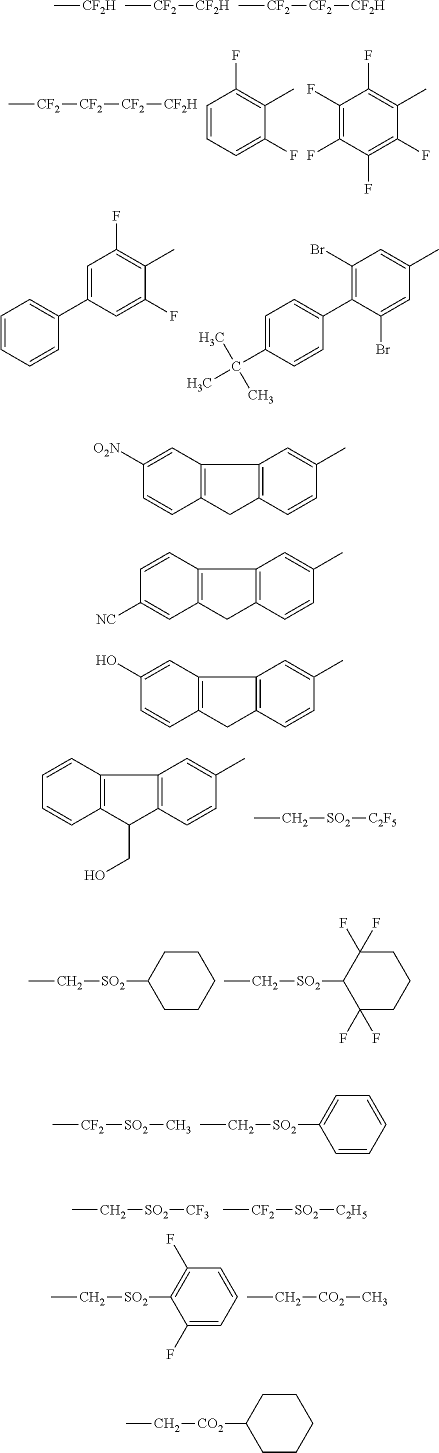

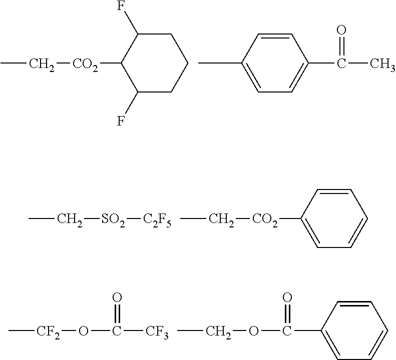

When the base generated by irradiation is the base represented by the formula (IB), Compound (D) is preferably a salt represented by the formula (ID):

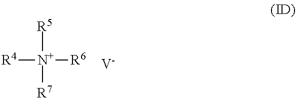

wherein R⁴, R⁵ and R⁶ each independently represent a C1-C12 hydrocarbon group which can have one or more substituents, and two or three selected from the group consisting of R⁴, R⁵ and R⁶ can be bonded each other to form a ring together with the nitrogen atom to which they are bonded, R⁷ represents an organic group having an aromatic hydrocarbon group, and V⁻ represents an organic counter anion.

Examples of the C1-C12 hydrocarbon group include the same as described above. Examples of the substituents of the C1-C12 hydrocarbon group include the same as described above. Examples of the substituted C1-C12 hydrocarbon group include the same as described above.

The aromatic hydrocarbon group can have one or more substituents, and examples thereof include a halogen atom, a hydroxyl group, a cyano group, a C1-C6 alkoxy group, a C1-C6 alkyl group, a 1-C6 hydroxyalkyl group, and a nitro group, and a C1-C6 alkoxy group is preferable and a methoxy group is more preferable. The organic group can have one or more connecting groups such as —SO₂—, —CO—, —O—CO— and —CO—O—.

Examples of the organic group having an aromatic hydrocarbon group include a phenyl group, a biphenyl group, a fluorenyl group, a naphthyl group, an anthryl group, a benzyl group, a phenylethyl group, and the followings.

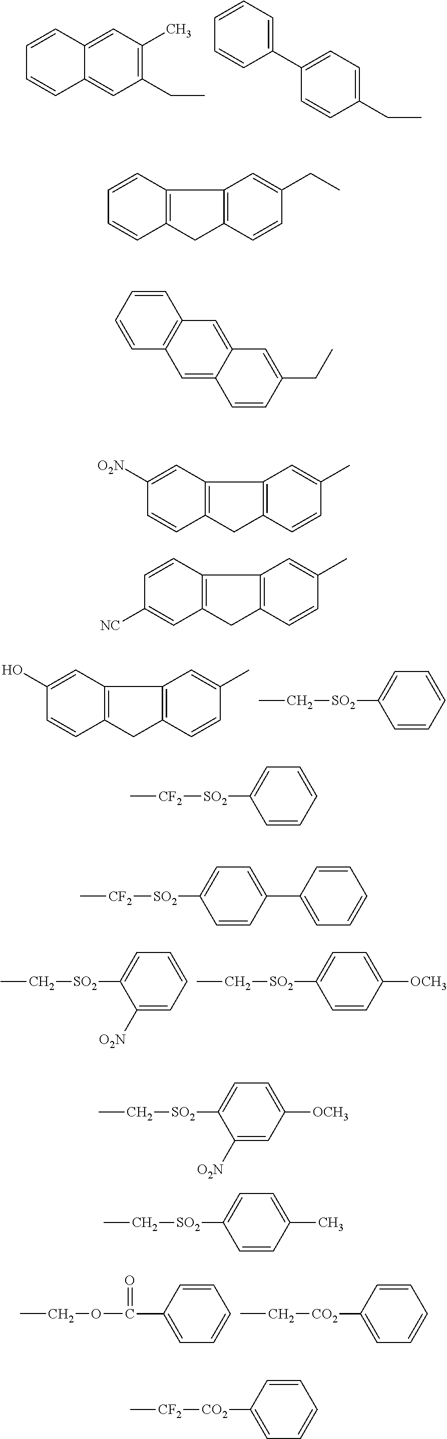

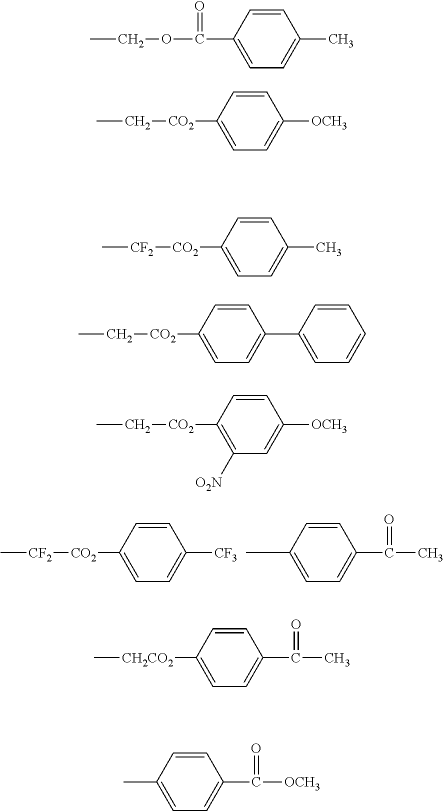

R⁷ is preferably a benzyl group or a phenylethyl group.

Examples of the cation of the salt represented by the formula (ID) include

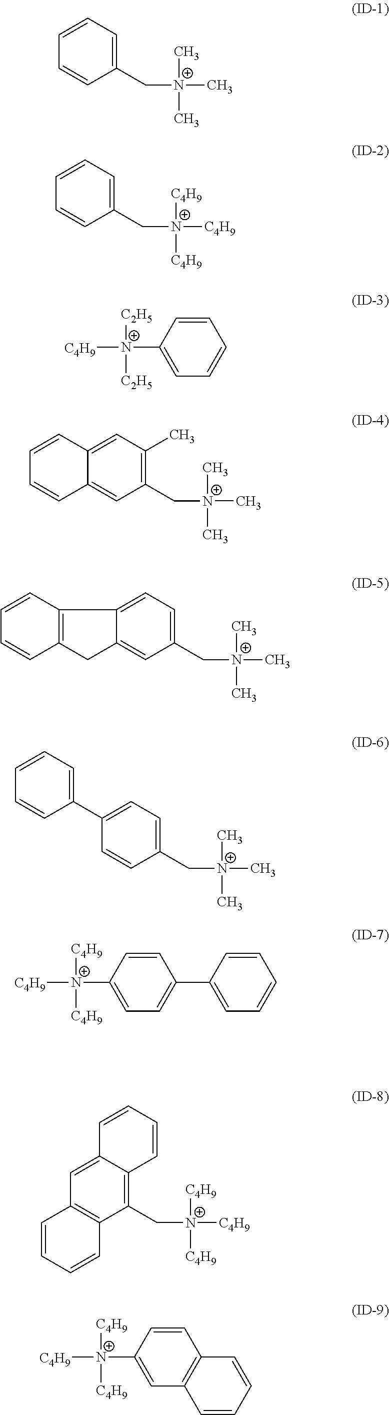

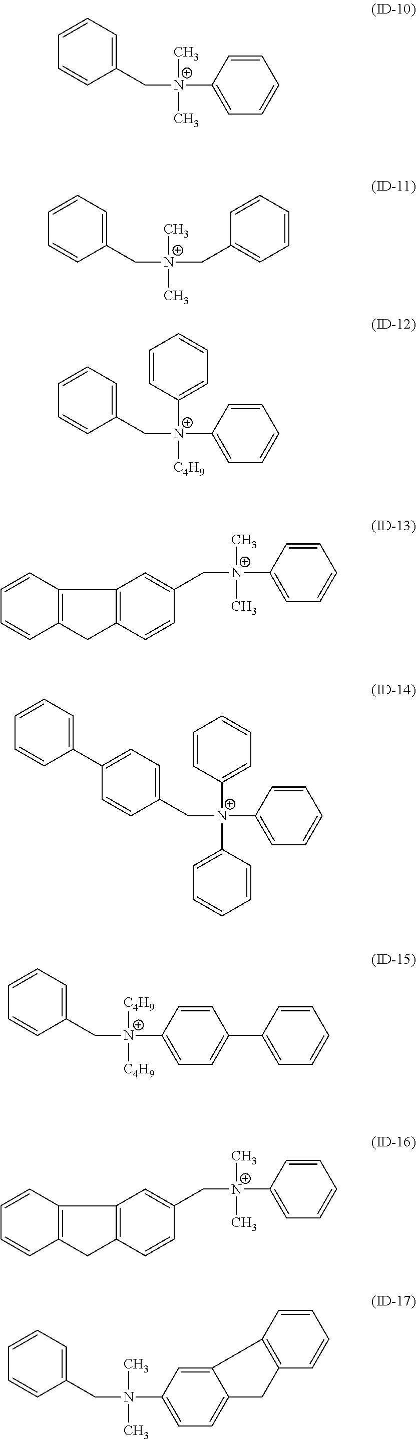

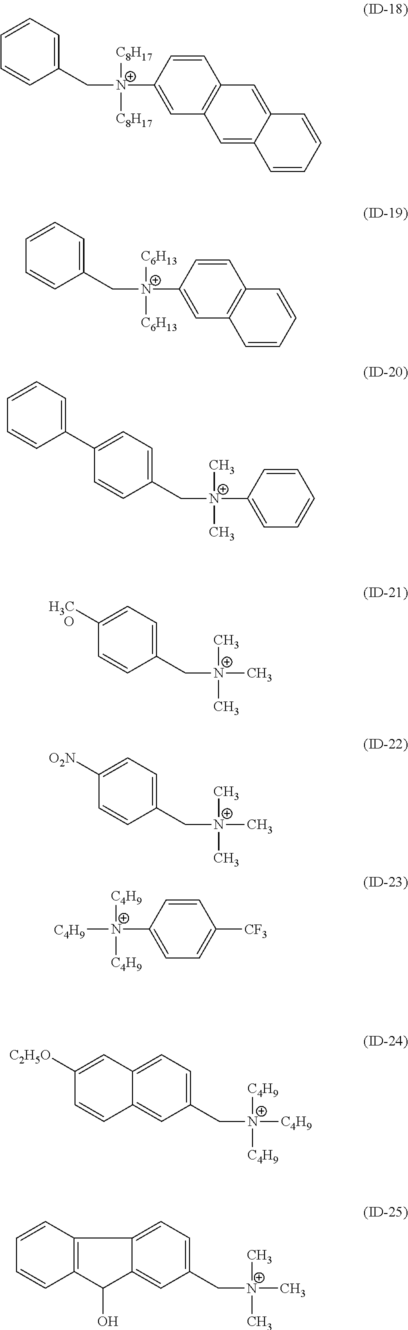

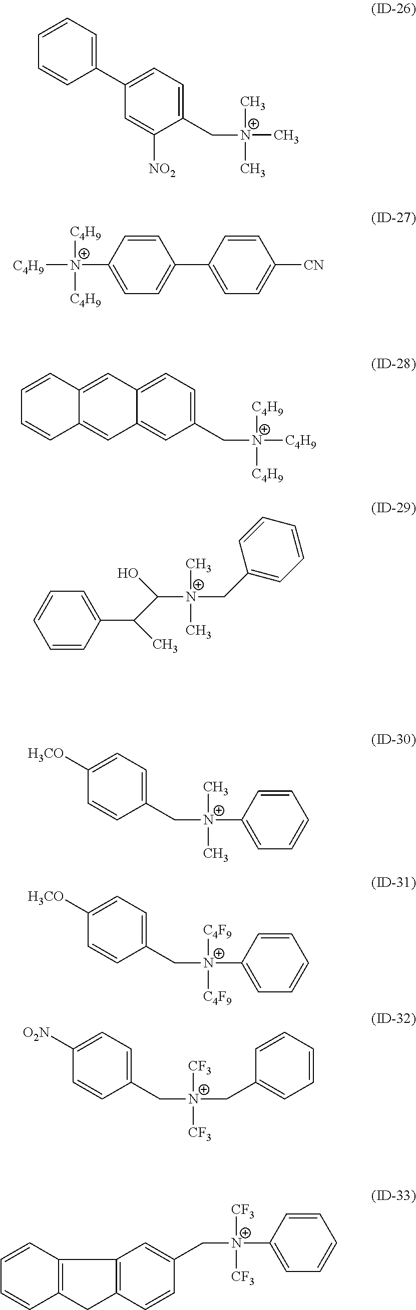

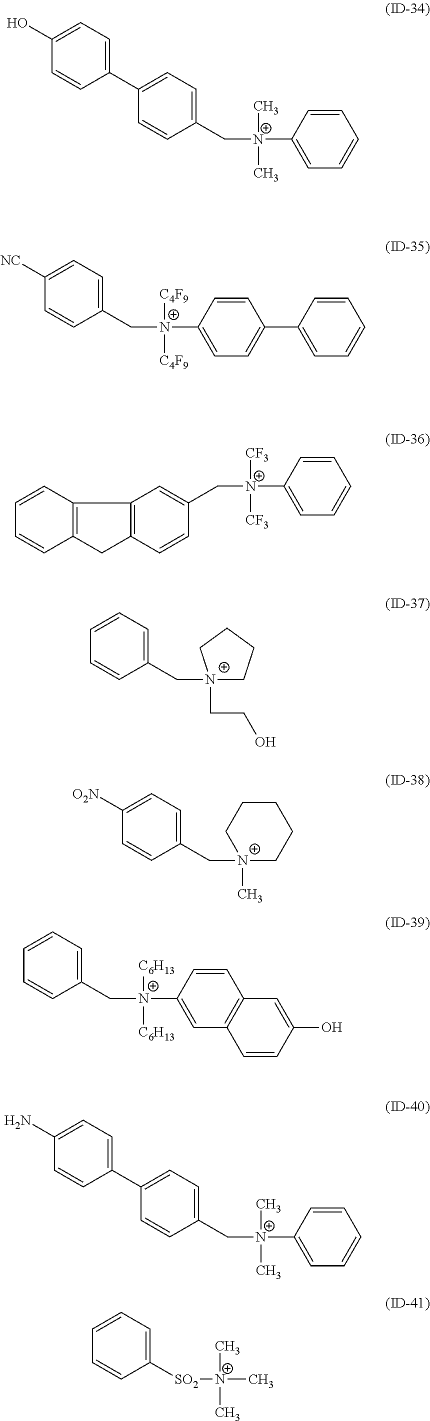

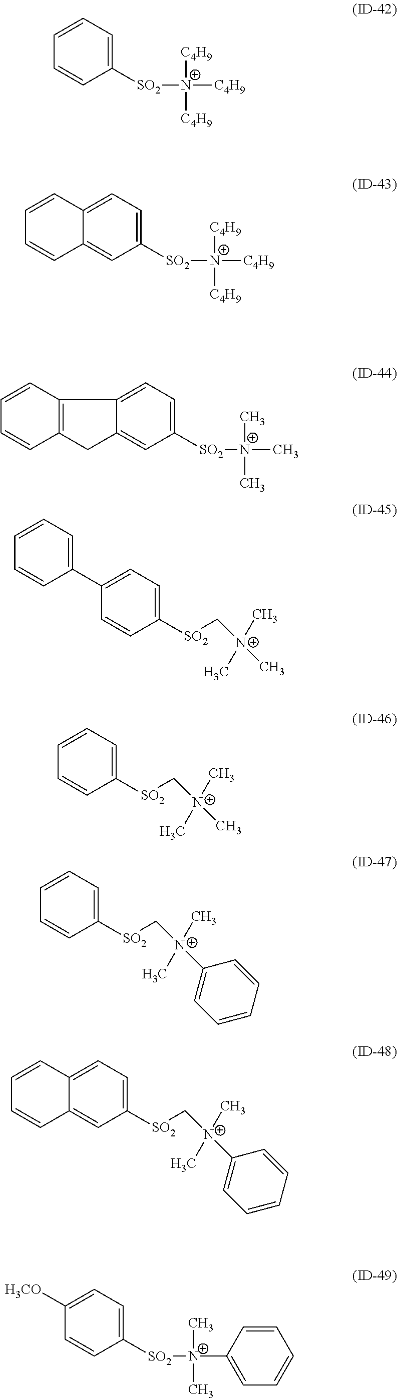

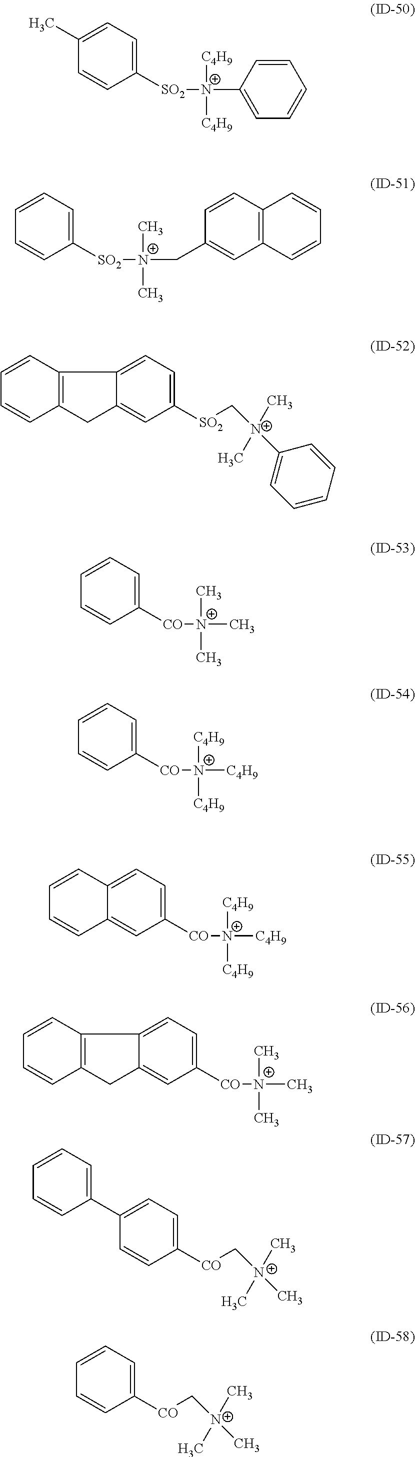

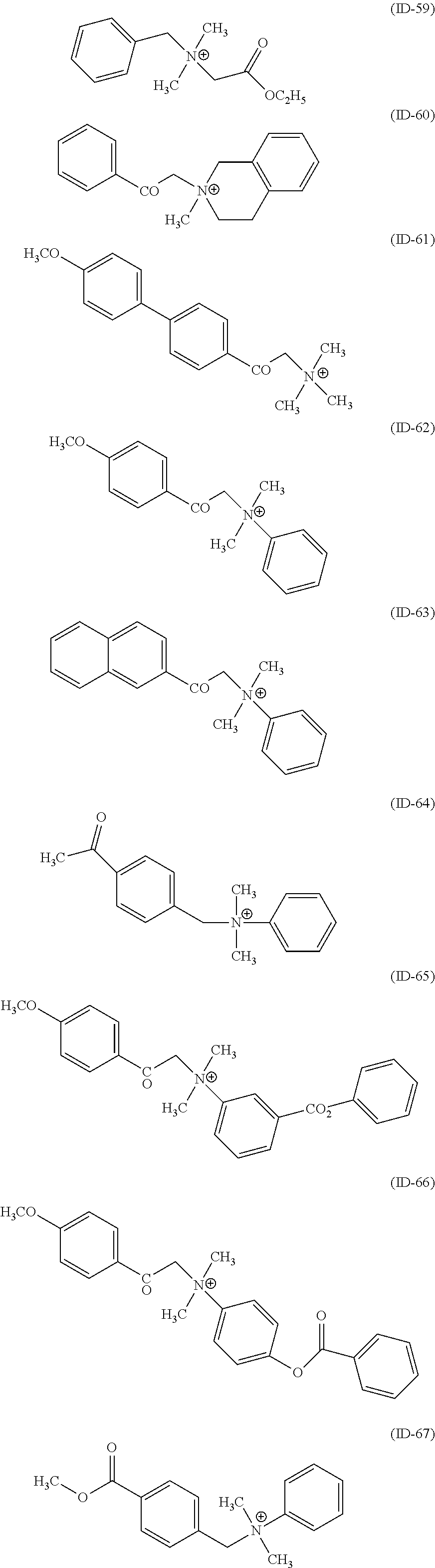

The acid generated by irradiation is preferably an acid represented by the formula (IA):

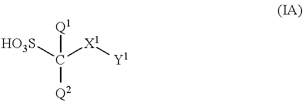

wherein Q¹ and Q² each independently represent a fluorine atom or a C1-C6 perfluoroalkyl group, X¹ represents a single bond or a C1-C17 divalent saturated hydrocarbon group in which one or more —CH₂— can be replaced by —O— or —CO—,
Y¹ represents a C1-C36 aliphatic hydrocarbon group which can have one or more substituents, a C3-C36 saturated cyclic hydrocarbon group which can have one or more substituents, or a C6-C36 aromatic hydrocarbon group which can have one or more substituents, and one or more —CH₂— in the aliphatic hydrocarbon group, the saturated cyclic hydrocarbon group and the aromatic hydrocarbon group can be replaced by —O— or —CO—.

Examples of the C1-C6 perfluoroalkyl group include a trifluoromethyl group, a pentafluoroethyl group, a heptafluoropropyl group, a nonafluorobutyl group, an undecafluoropentyl group and a tridecafluorohexyl group, and a trifluoromethyl group is preferable. Q¹ and Q² each independently preferably represent a fluorine atom or a trifluoromethyl group, and Q¹ and Q² are more preferably fluorine atoms.

Examples of the C1-C17 divalent saturated hydrocarbon group include a C1-C17 alkylene group and a divalent group having an alicyclic divalent hydrocarbon group. Examples of the alkylene group include a linear alkanediyl group such as a methylene group, an ethylene group, a propane-1,3-diyl group, a butane-1,4-diyl group, a pentane-1,5-diyl, a hexane-1,6-diyl group, a heptane-1,7-diyl group, an octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group and a heptadecane-1,17-diyl group,

a branched chain alkanediyl group formed by replacing one or more hydrogen atom of the above-mentioned linear alkanediyl group by a C1-C4 alkyl group, and
a divalent group having an alicyclic divalent hydrocarbon group such as the following groups represented by the formulae (X¹-A) to (X¹-C):

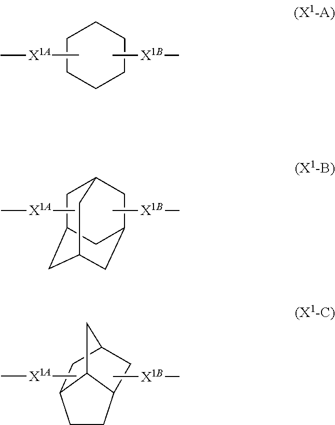

wherein X^{1A} and X^{1B} independently each represent a C1-C6 alkylene group in which one or more —CH₂— can be replaced by —O— or —CO—, with the proviso that total carbon number of the group represented by the formula (X¹-A), (X¹-B) or (X¹-C) is 1 to 17.

Examples of the C1-C17 saturated hydrocarbon group in which one or more —CH₂— are replaced by —O— or —CO— include *—CO—O—X¹⁰—, *—O—X¹¹—CO—O—, *—X¹⁰—O—CO— and *—X¹¹—O—X¹²— wherein X¹⁰ represents a single bond or a C1-C15 alkanediyl group, X¹¹ represents a C1-C15 alkanediyl group, X¹² represents a C1-C15 alkanediyl group, with proviso that total carbon number of X¹¹ and X¹² is 1 to 16 and * represents a binding position to —C(Q¹)(Q²)-.

Examples of X¹ include the followings.

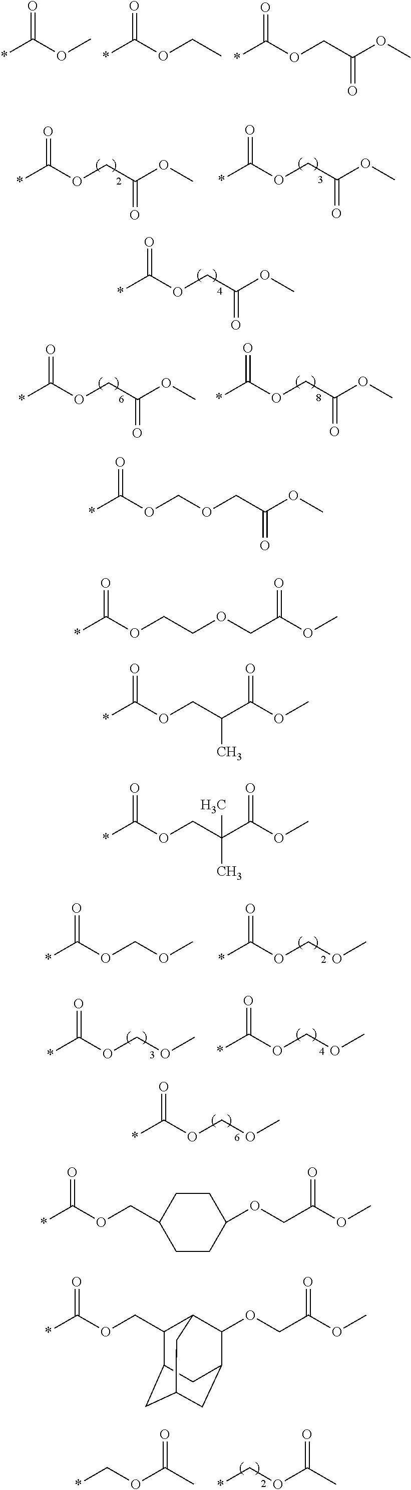

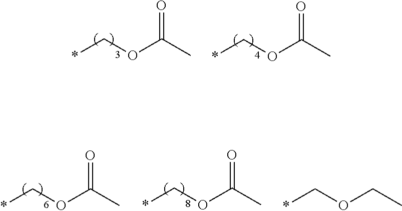

Examples of the substituent of Y¹ include a halogen atom, a C1-C12 aliphatic hydrocarbon group, a C6-C20 aromatic hydrocarbon group, a C7-C21 aralkyl group, a glycidyloxy group and a C2-C4 acyl group. Examples of the halogen atom include a fluorine atom, a chlorine atom, a bromine atom and an iodine atom. Examples of the aliphatic hydrocarbon group include an alkyl group such as a methyl group, an ethyl group, a propyl group, an isopropyl group, a butyl group, a sec-butyl group, a tert-butyl group, a pentyl group, a hexyl group, a heptyl group, a 2-ethylhexyl group, an octyl group, a nonyl group, a decyl group, an undecyl group and a dodecyl group. Examples of the acyl group include an acetyl group and a propionyl group. Examples of the aromatic hydrocarbon group include a phenyl group, a naphthyl group, an anthryl group, a p-methylphenyl group, a p-tert-butylphenyl group and a p-adamantylphenyl group, a tolyl group, a xylyl group, a cumyl group, a mesityl group, a biphenyl group, a phenanthryl group, a 2,6-diethylphenyl group, and a 2-methyl-6-ethylphenyl group. Examples of the aralkyl group include a benzyl group, a phenylethyl group, a phenylpropyl group, a trityl group, a naphthylmethyl group and a naphthylethyl group.

Preferable examples of Y¹ include the groups represented by the formulae (W1) to (W26):

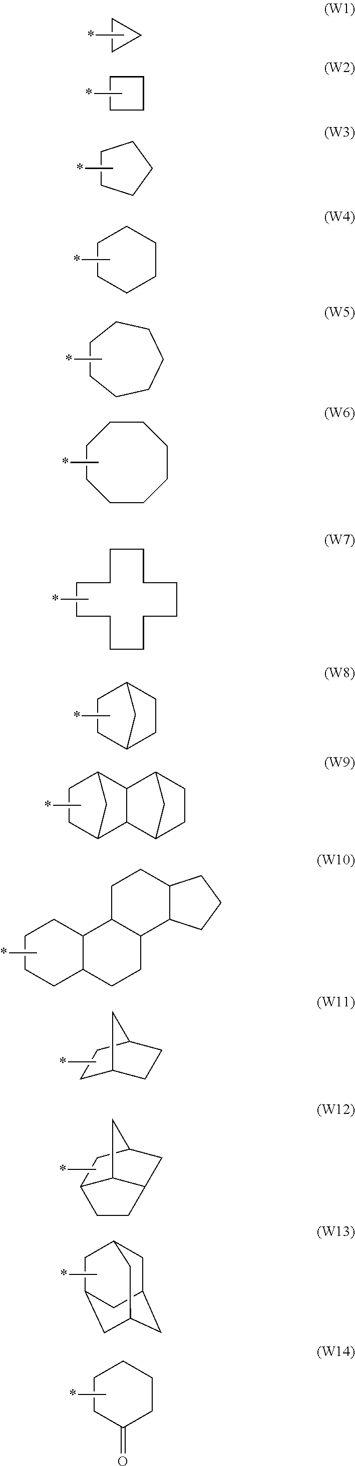

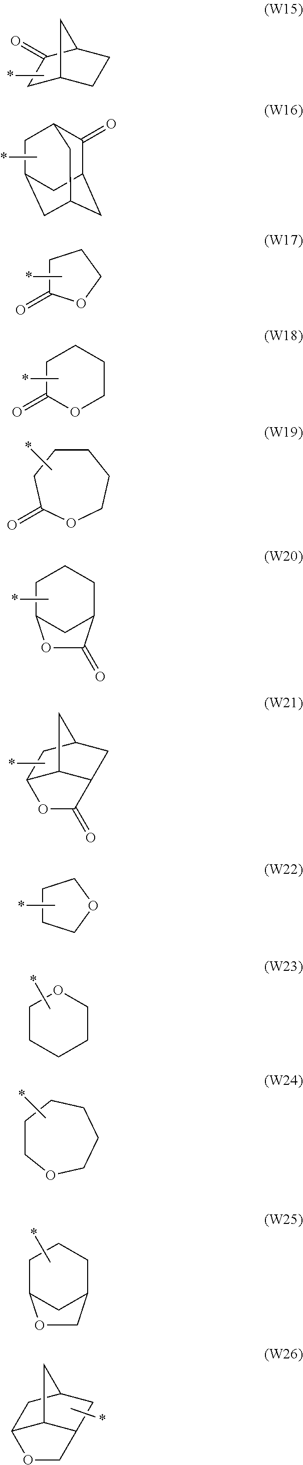

Other examples of Y¹ include a hydrocarbon group having a hydroxyl group or a group having a hydroxyl group other that a group having a lactone structure, a group having a lactone structure, a group having a ketone structure, a group having an aromatic hydrocarbon group or a group having an aromatic ring, and a group having an ether structure.

Y¹ is preferably a group represented by the formula (Y¹-1), (Y¹-2) or (Y¹-3):

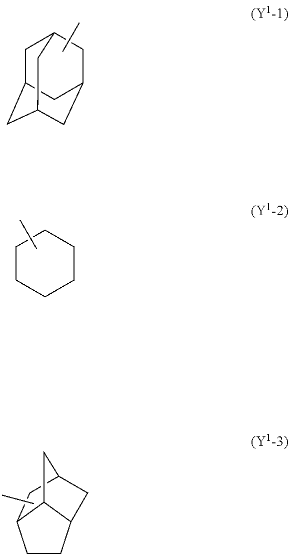

wherein one or more —CH₂— in the ring can be replaced by —O— or —CO—, and the ring can have one or more substituents. Examples of the substituent include the same as described in the substituents of Y¹.

Examples of Y¹ having one or more substituents include the followings:

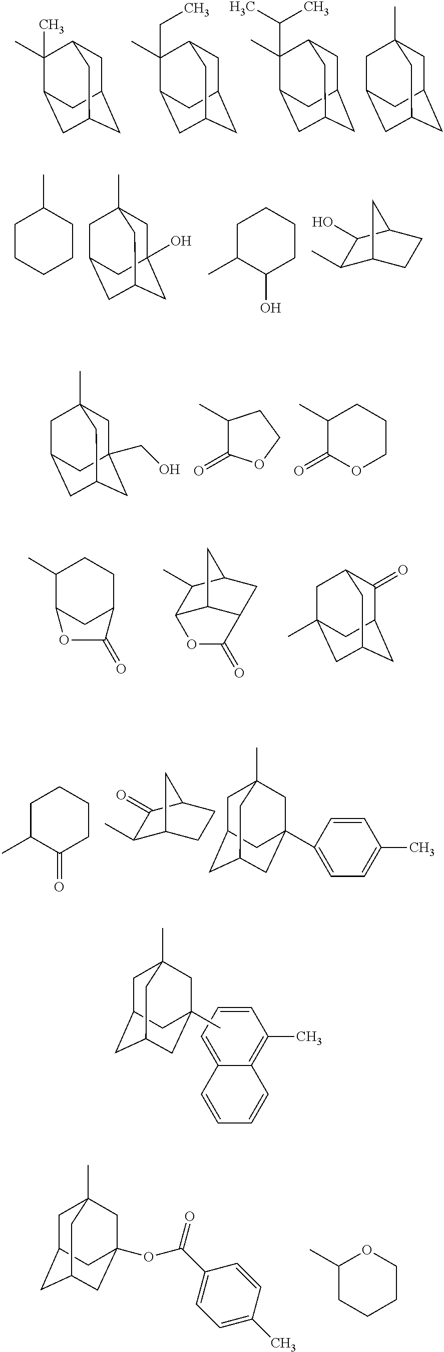

Examples of the acid represented by the formula (IA) include acids represented by the formulae (IIA), (IIB), (IIC) and (IID), and the acids represented by the formulae (IIA) and (IIB) are preferable.

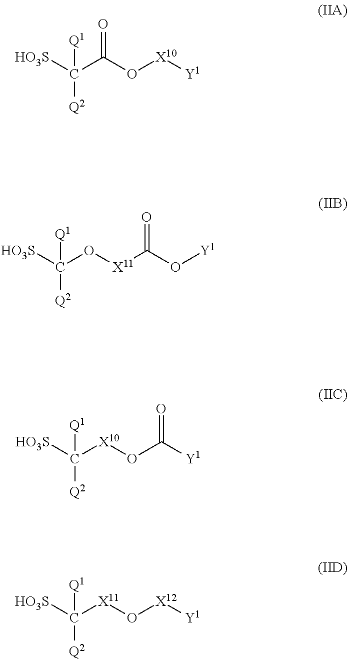

wherein Q¹, Q², X¹⁰, X¹¹, X¹² and Y¹ are the same as defined above.

Examples of the acid represented by the formula (IIA) include the followings.

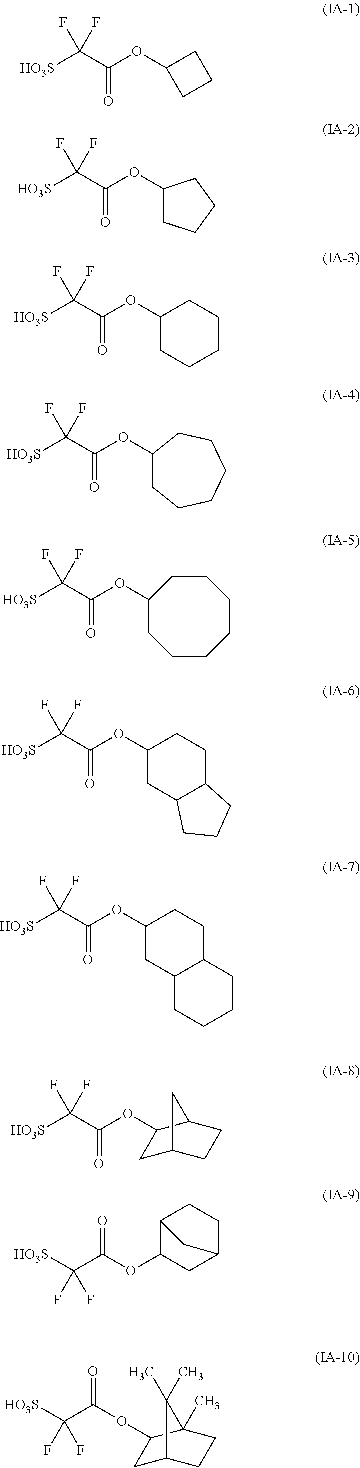

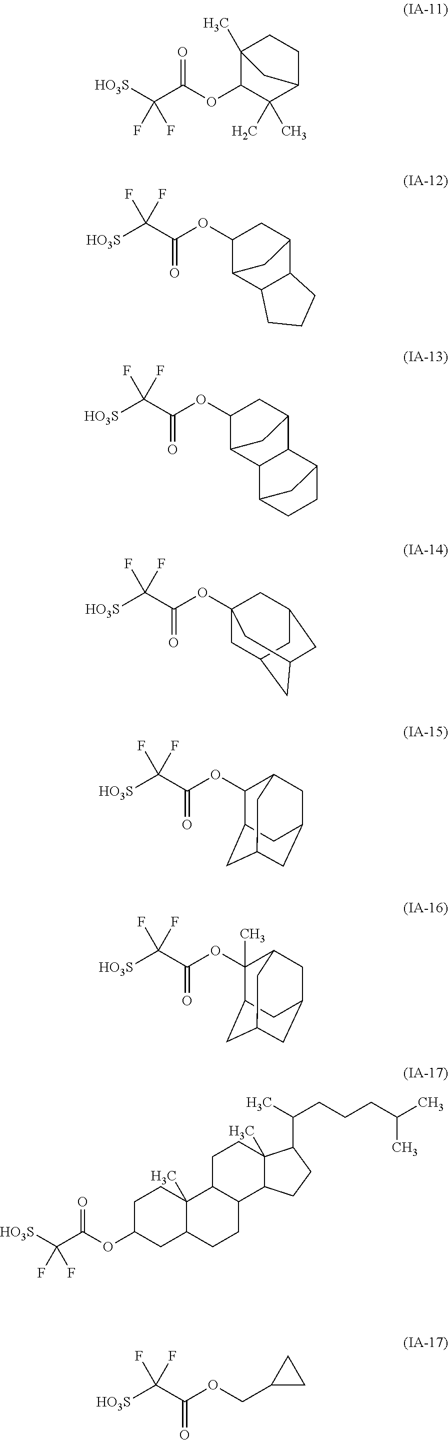

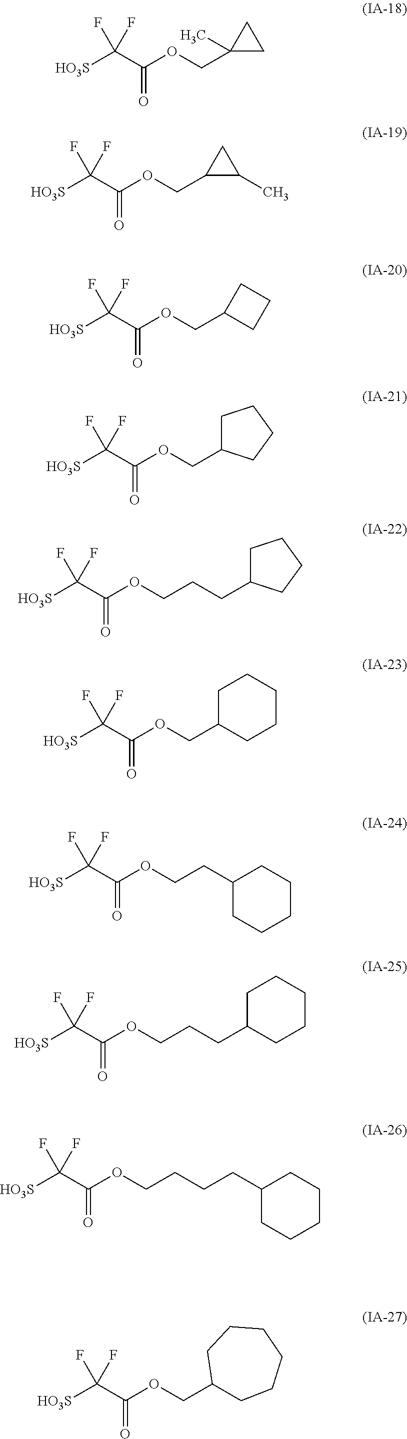

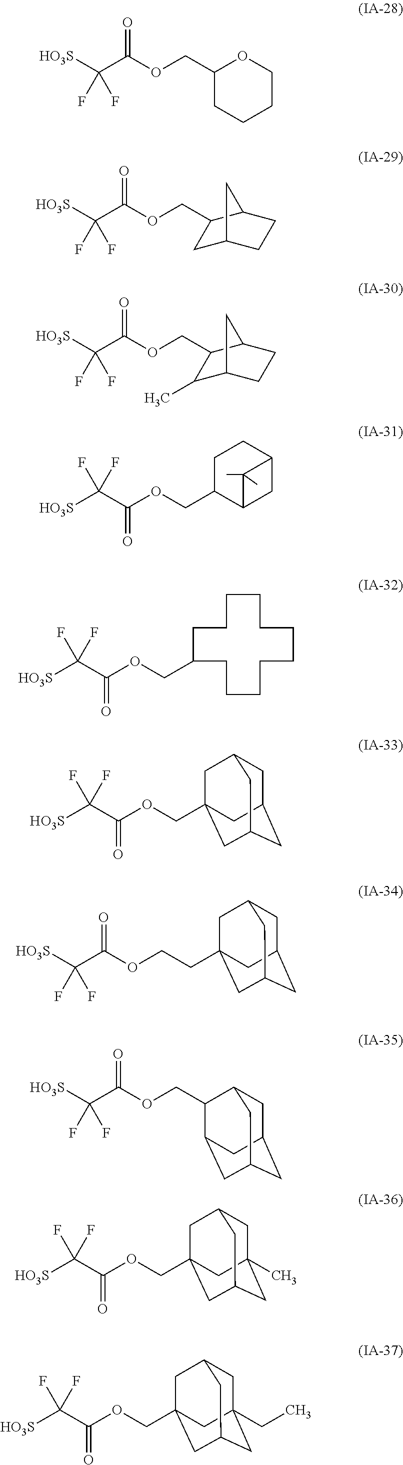

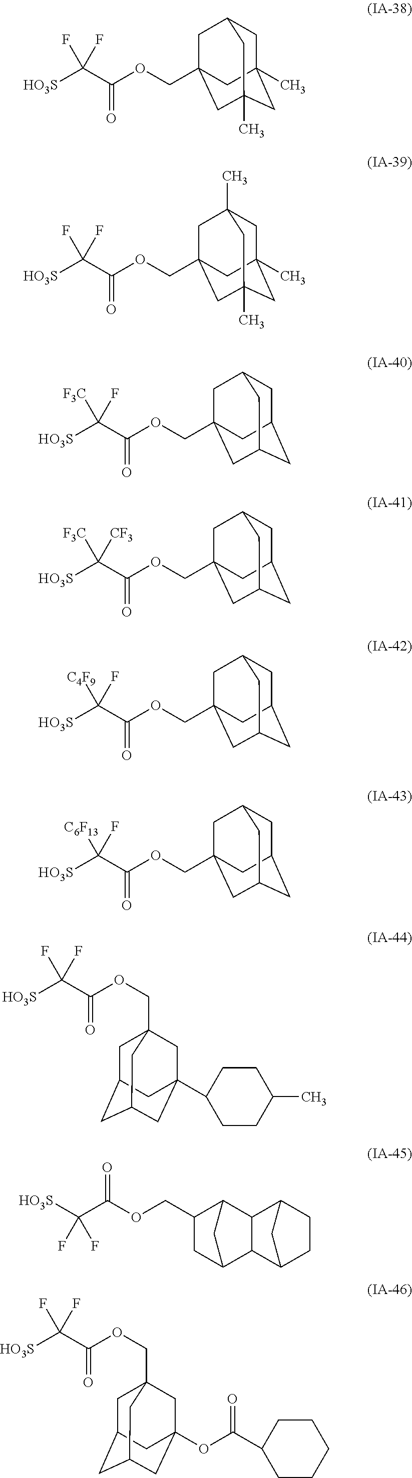

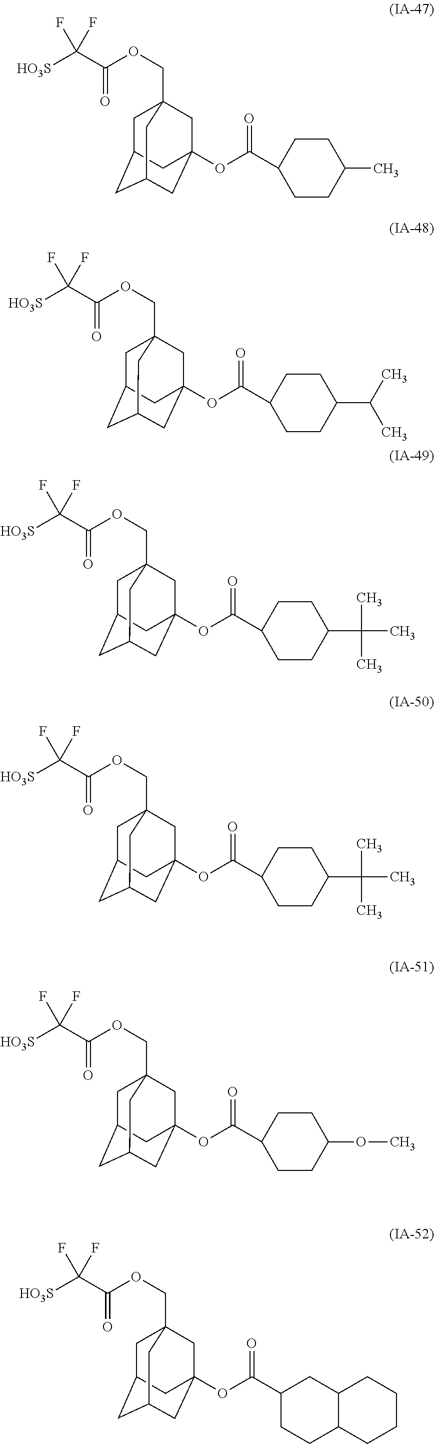

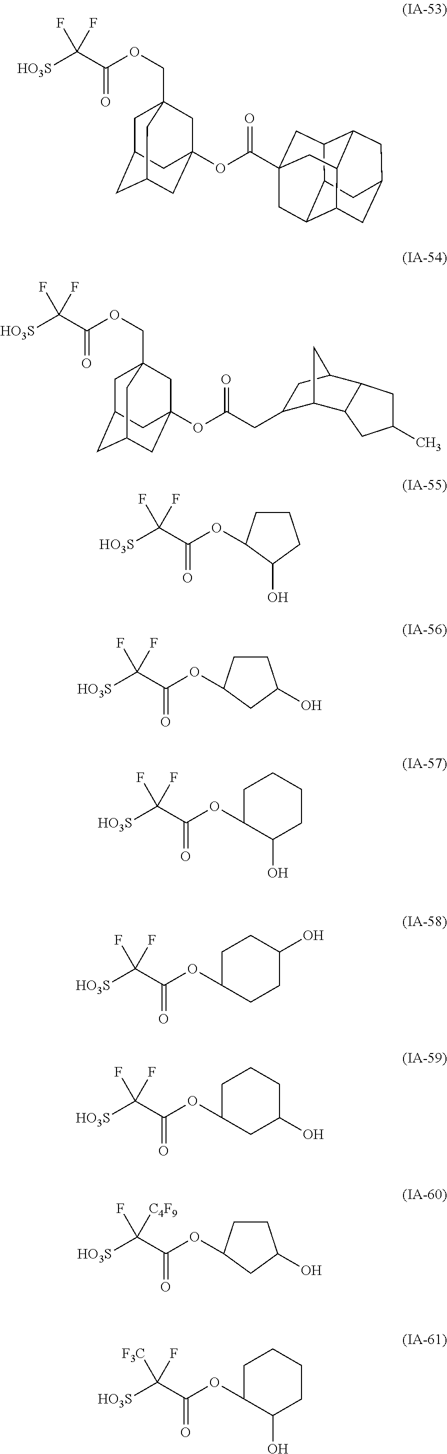

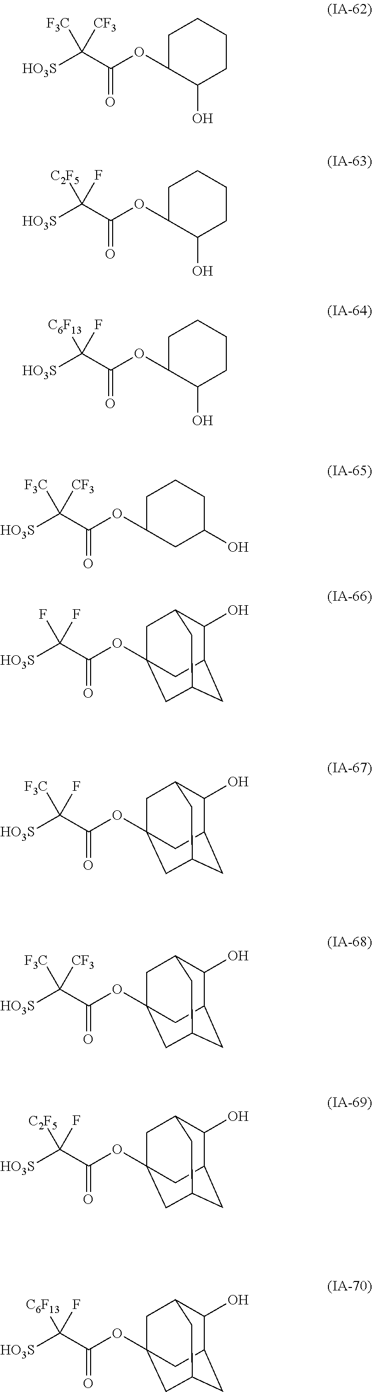

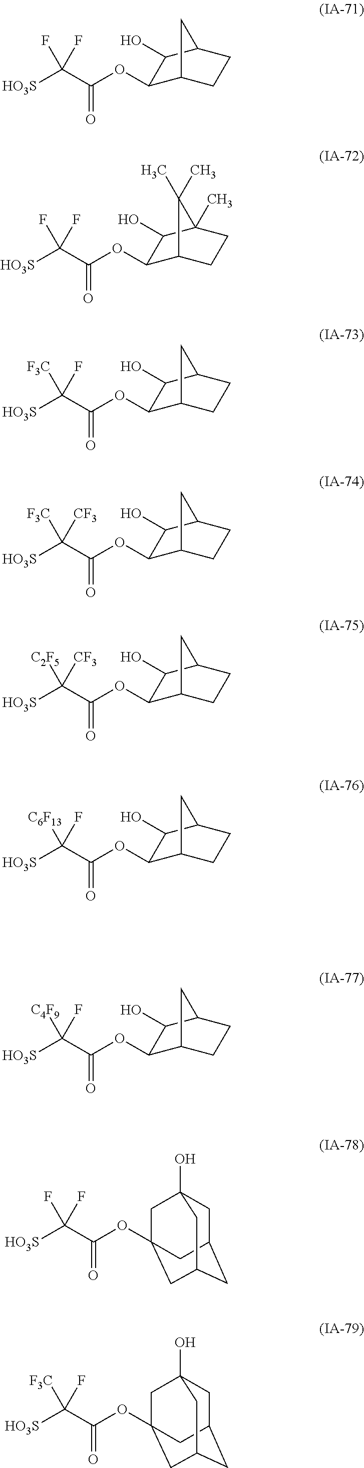

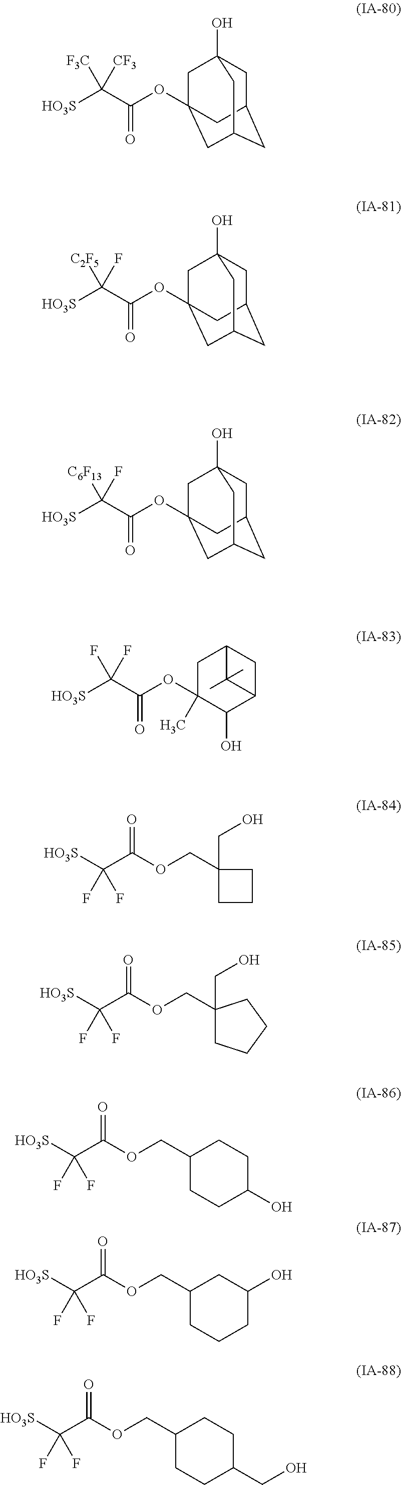

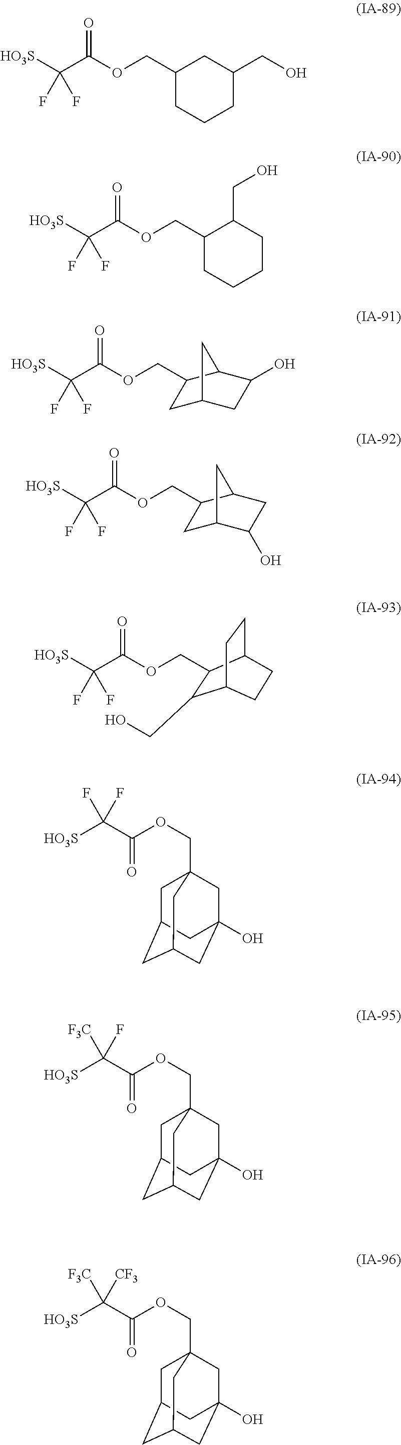

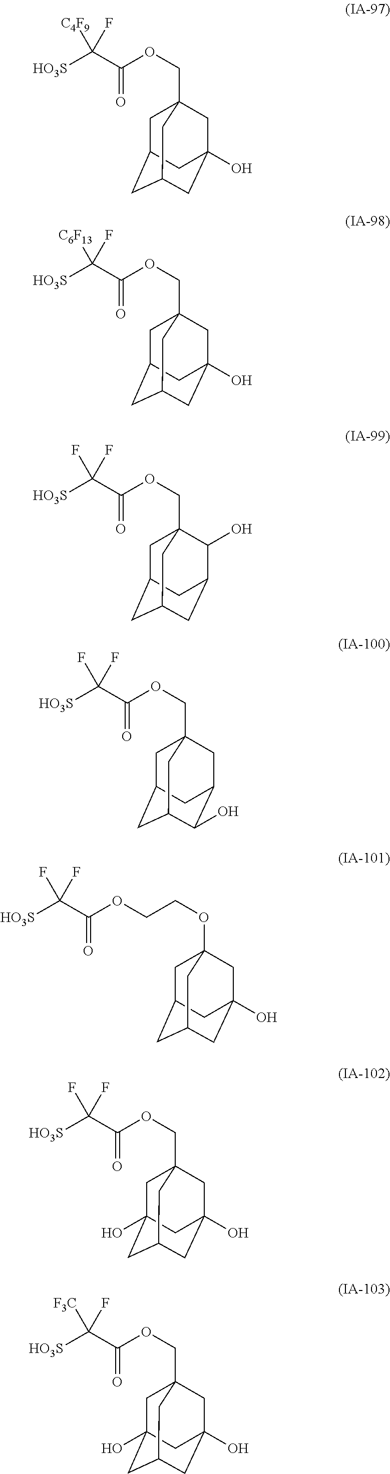

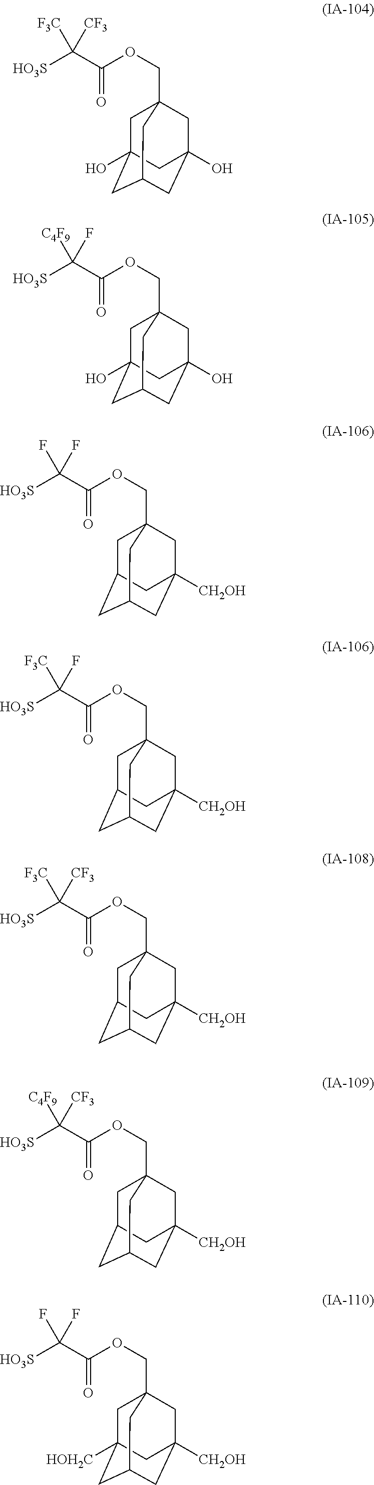

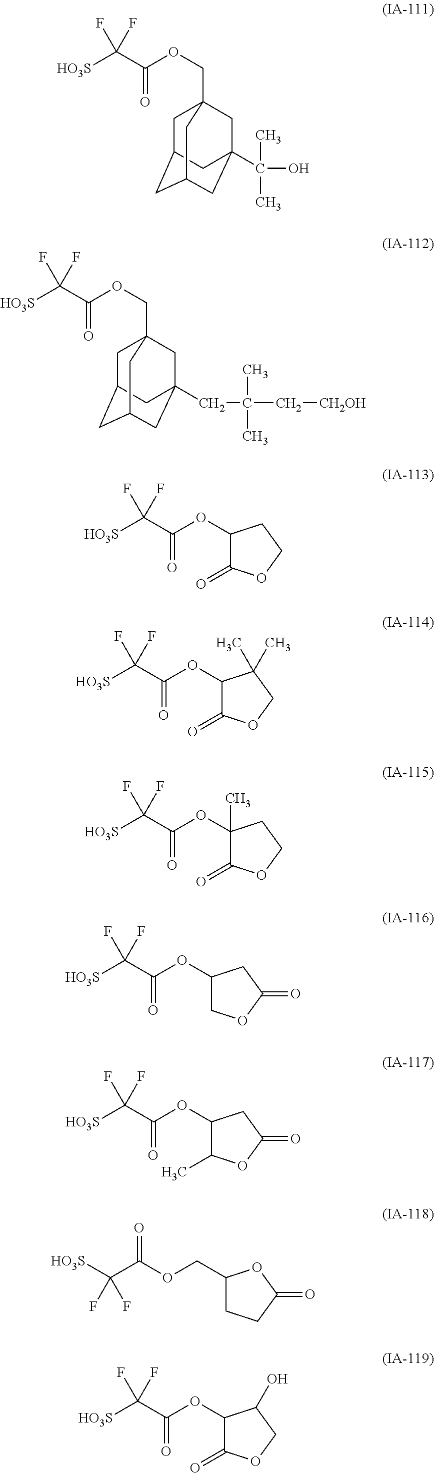

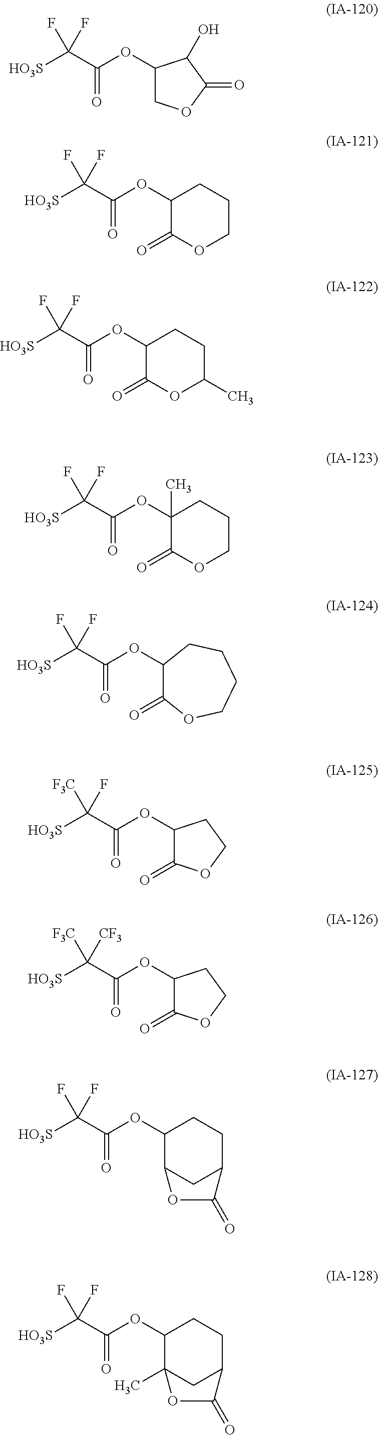

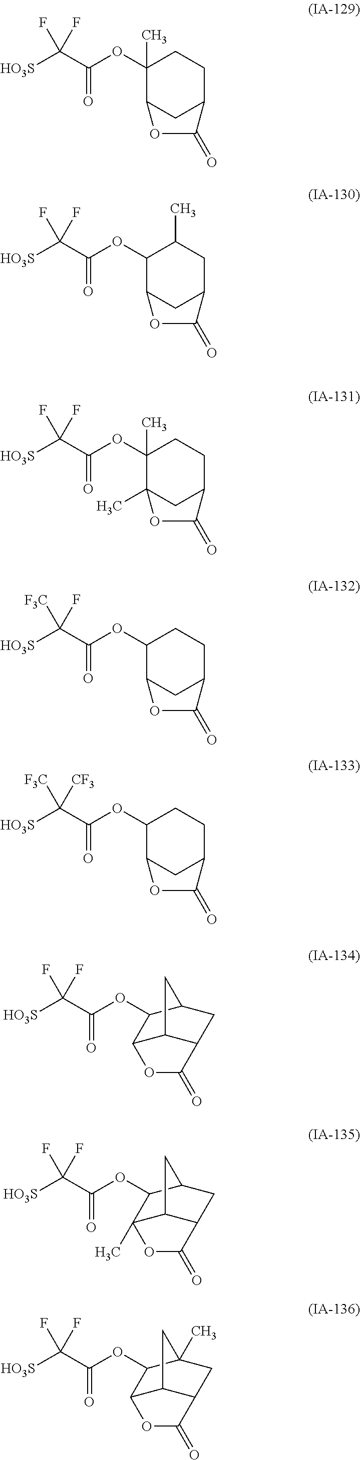

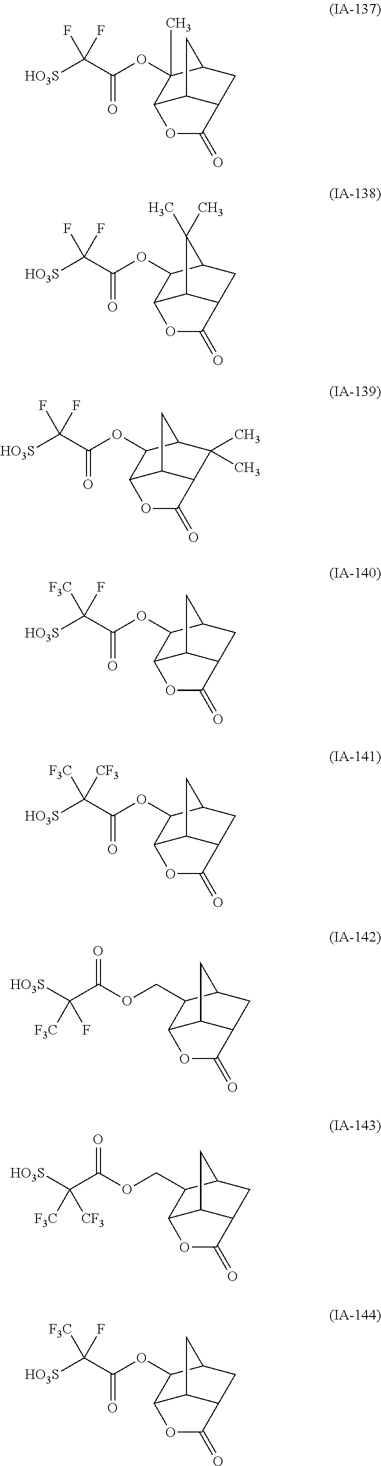

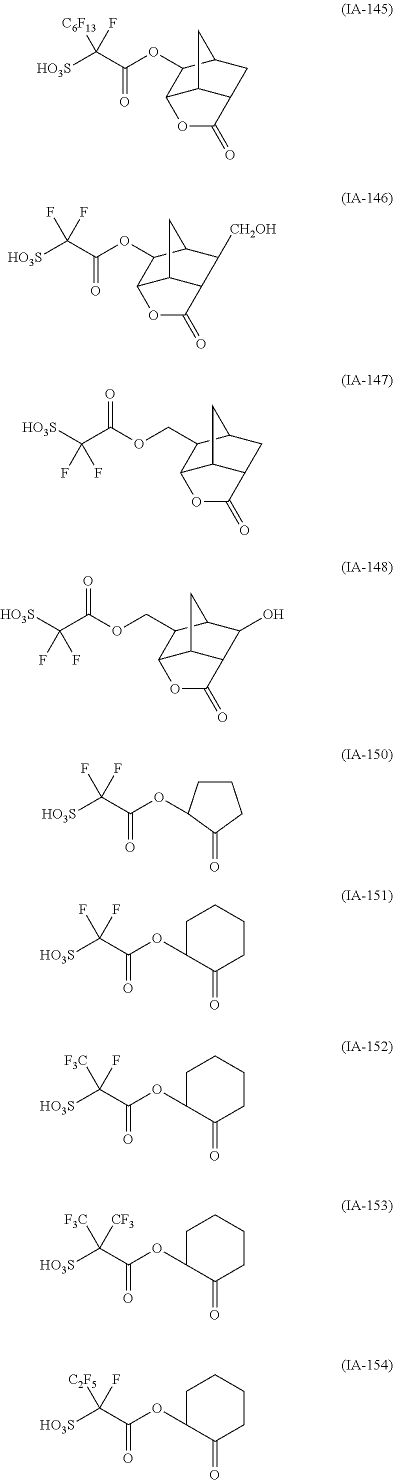

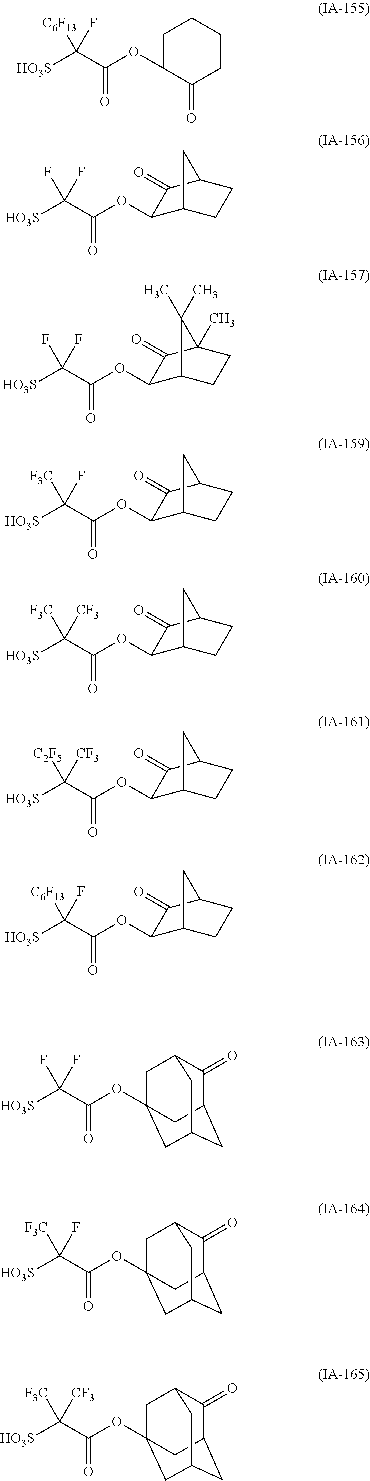

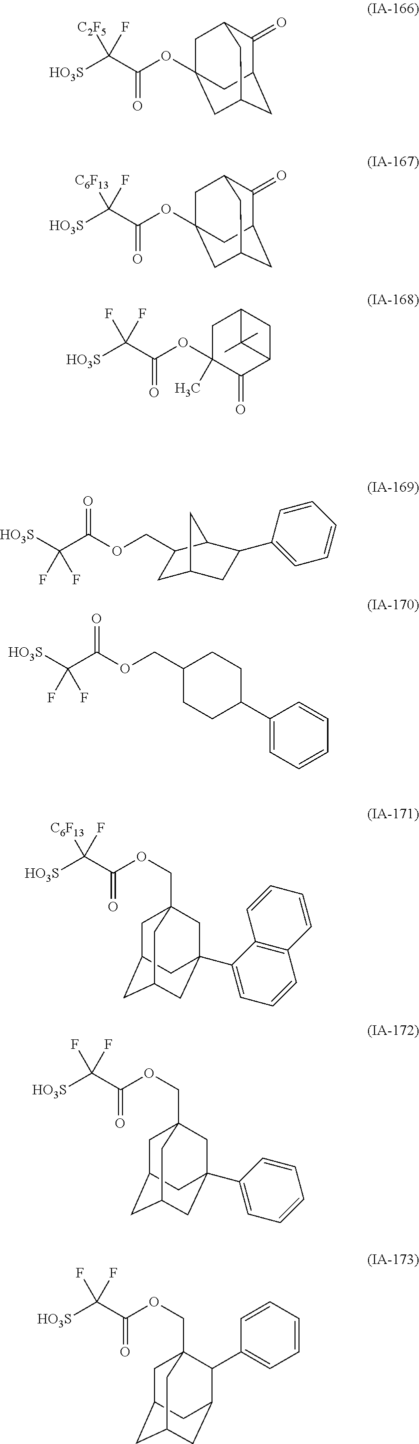

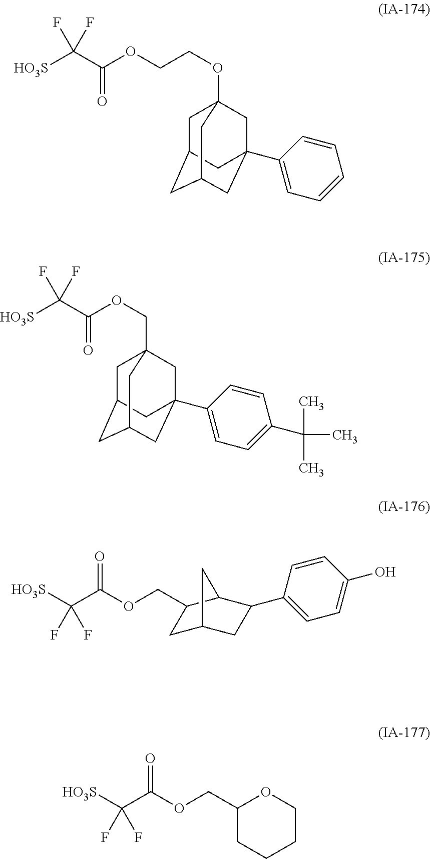

Examples of the acid represented by the formula (IIB) include the followings.

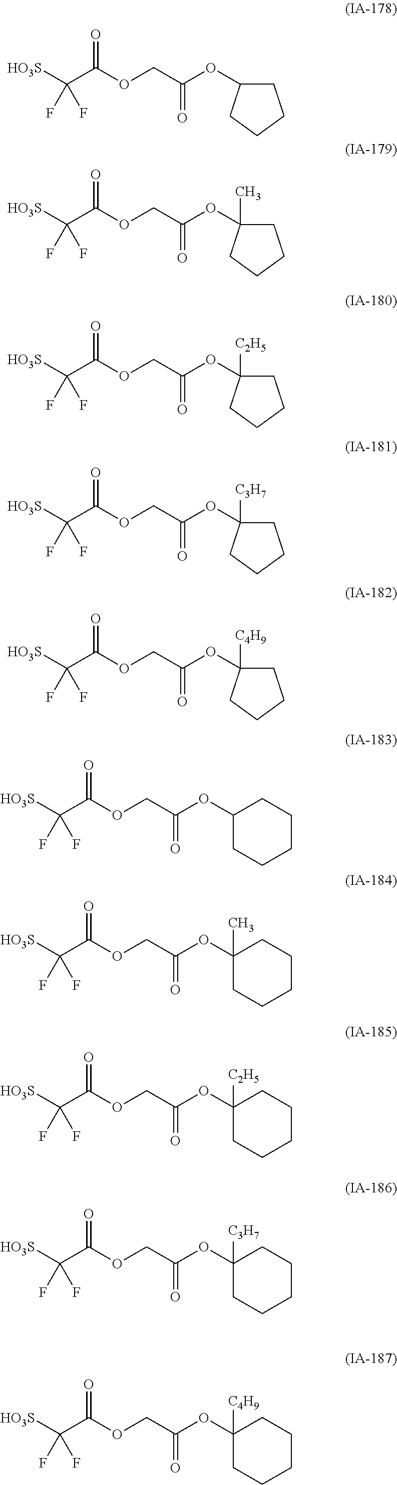

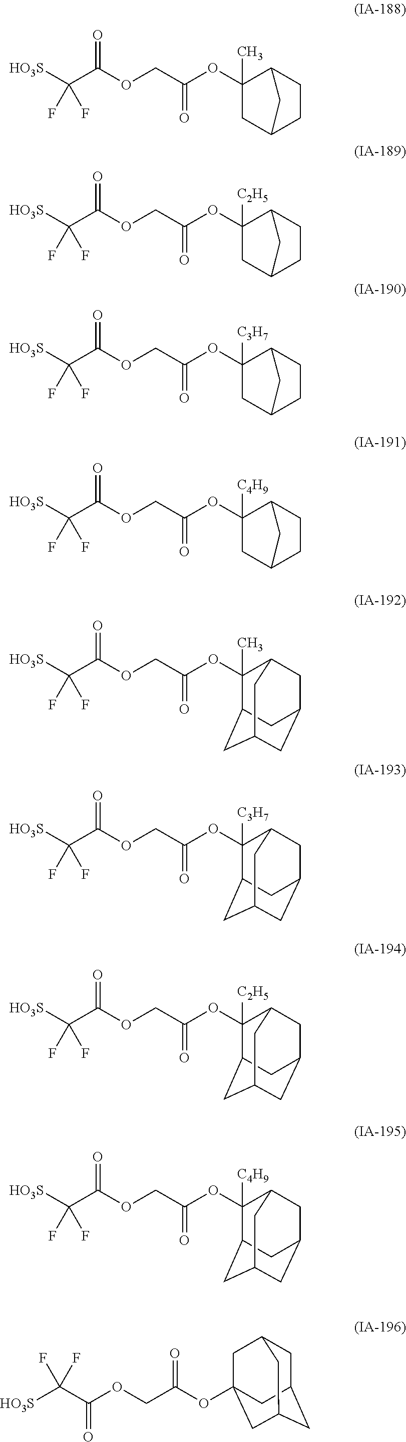

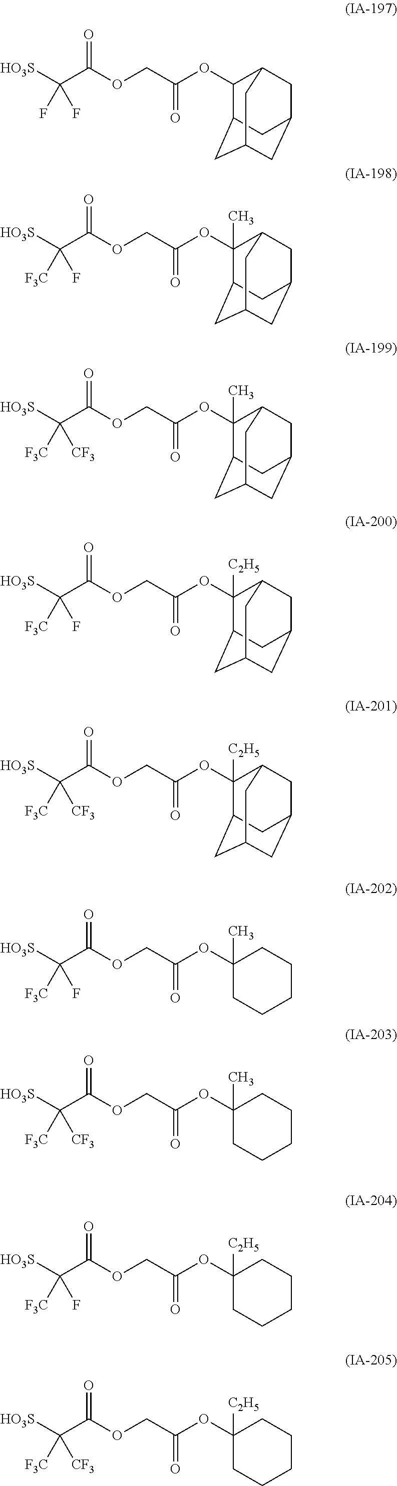

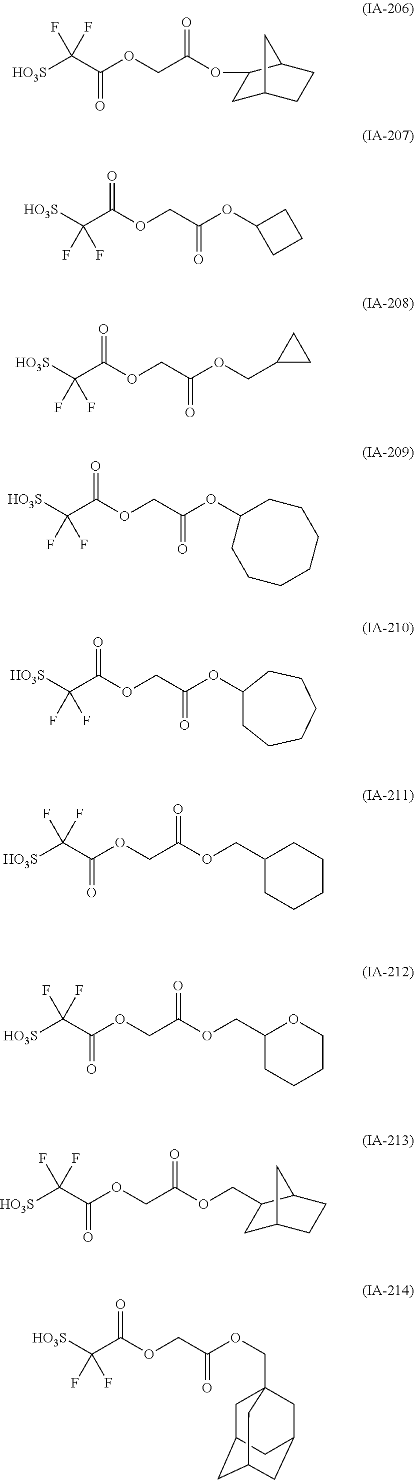

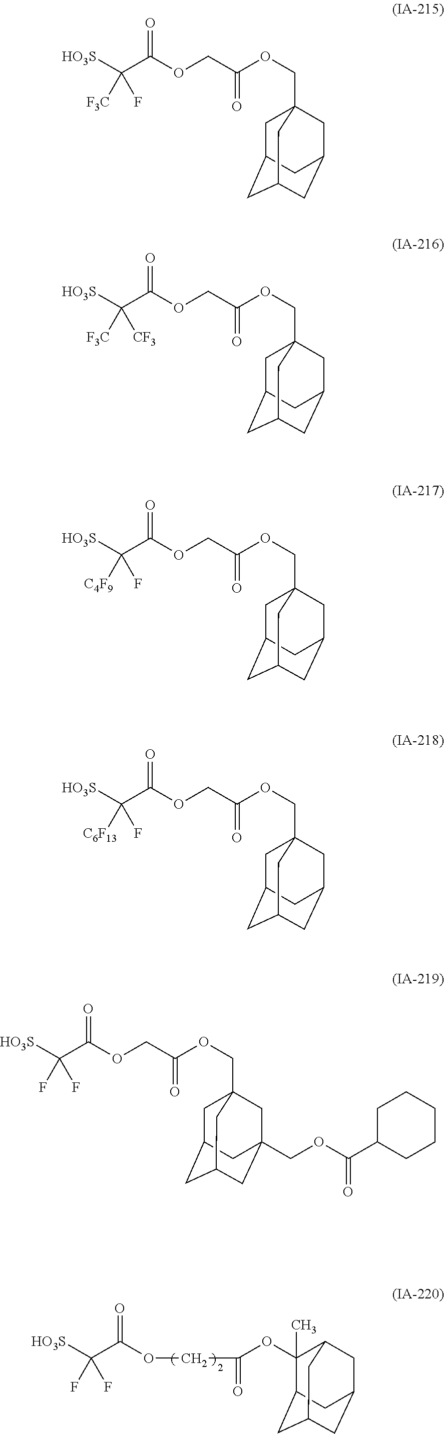

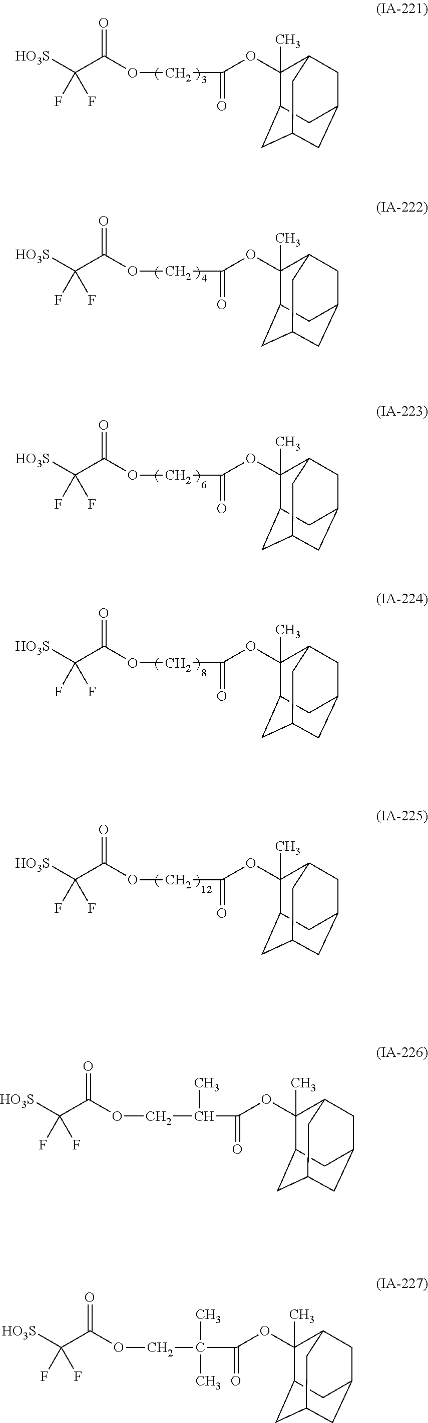

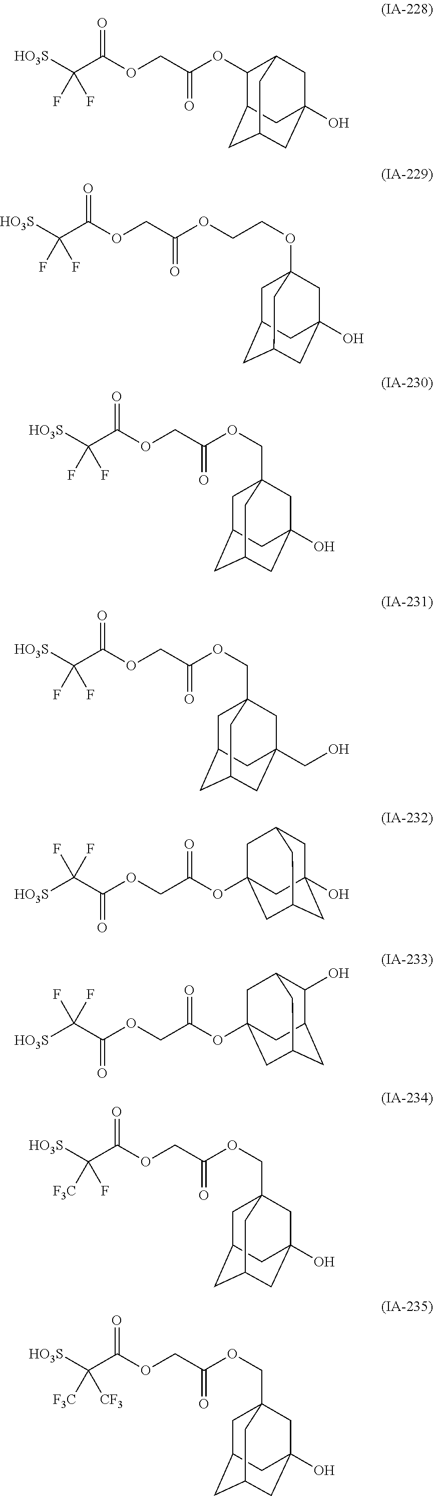

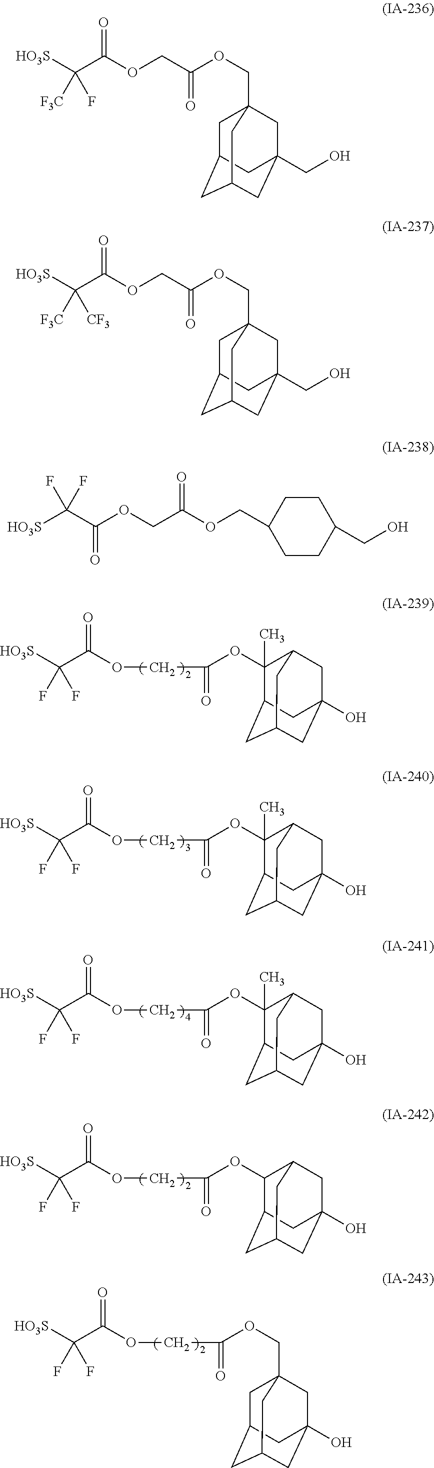

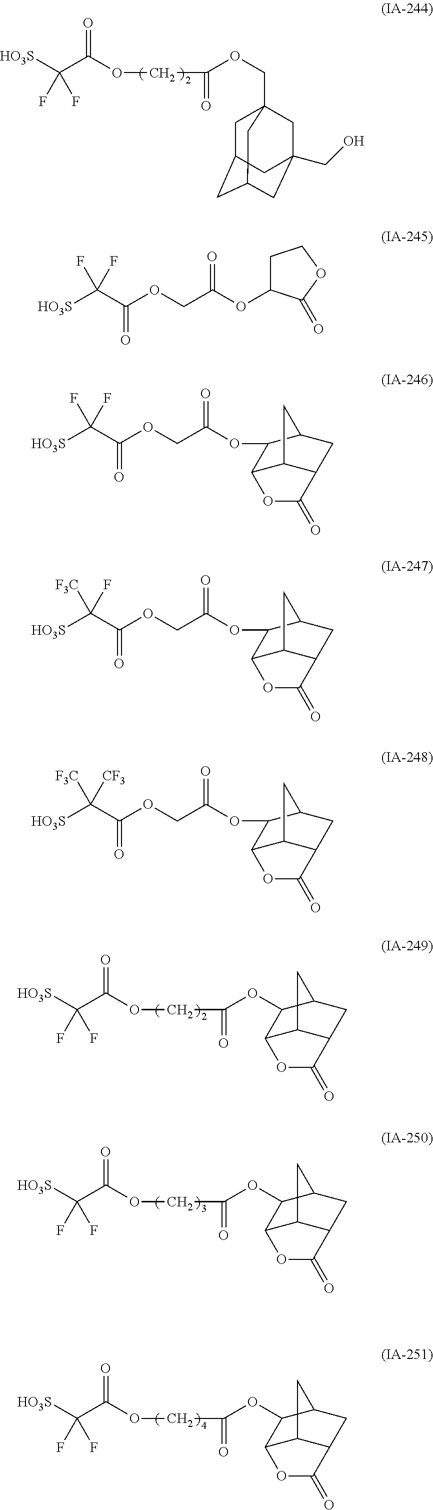

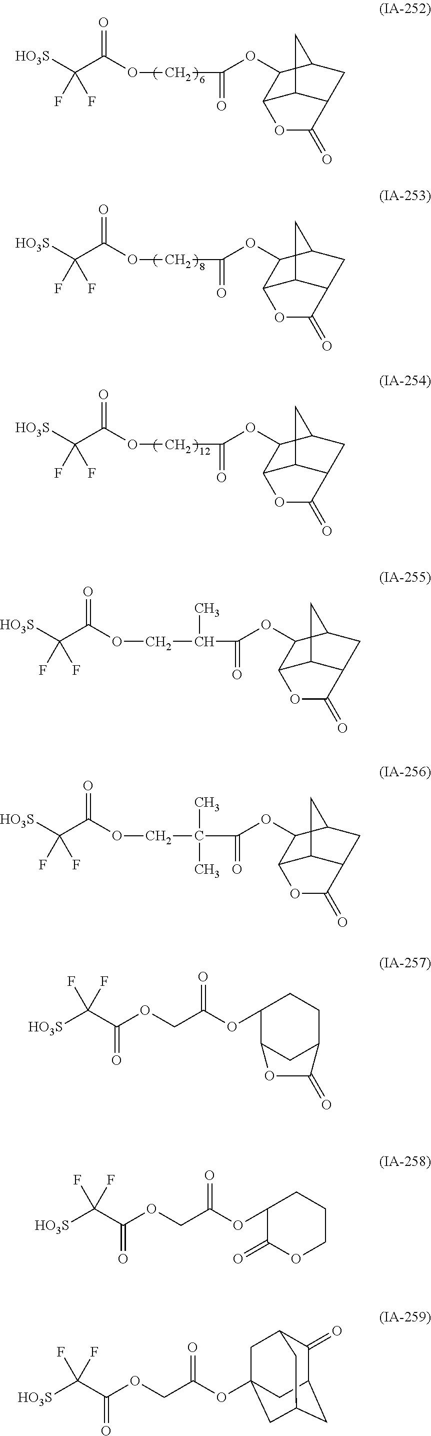

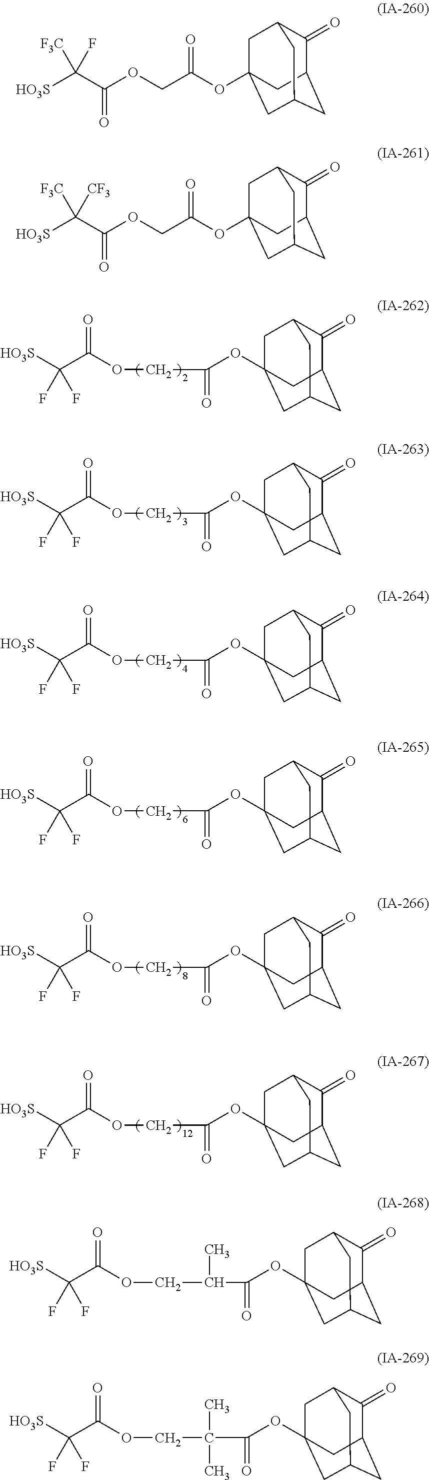

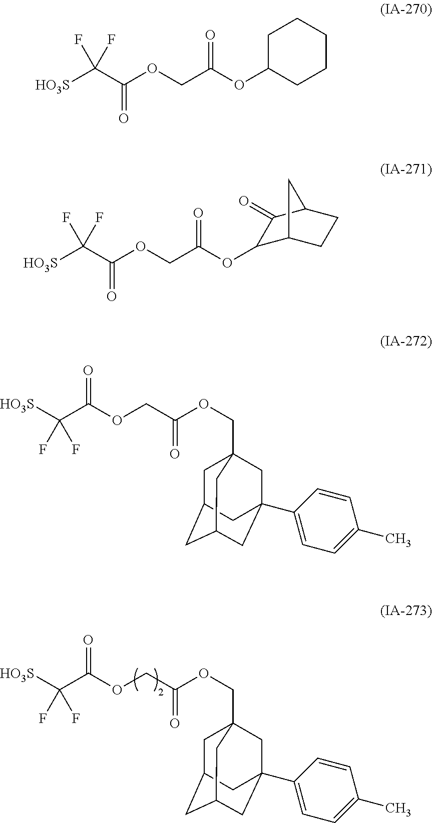

Examples of the acid represented by the formula (IIC) include the followings.

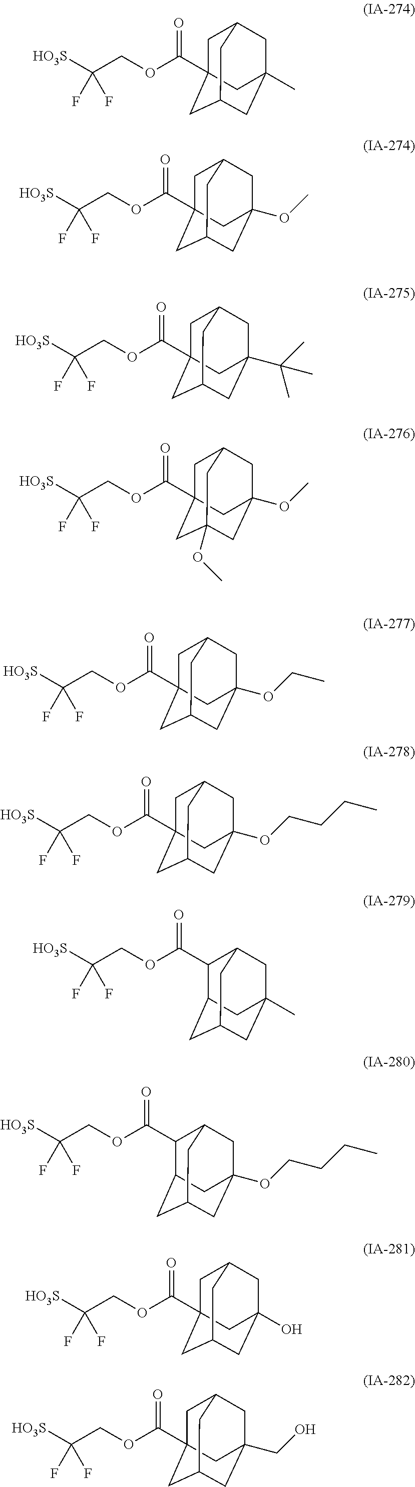

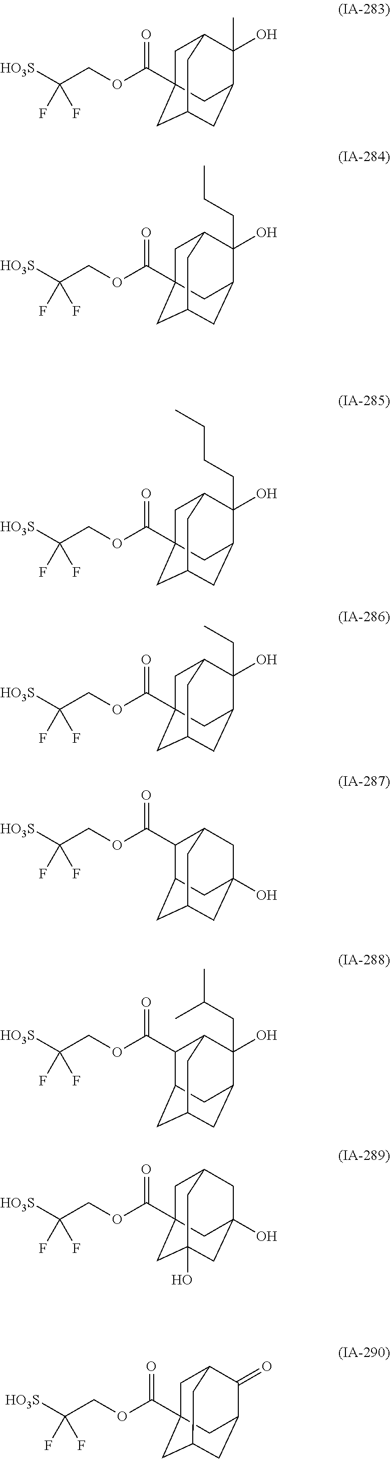

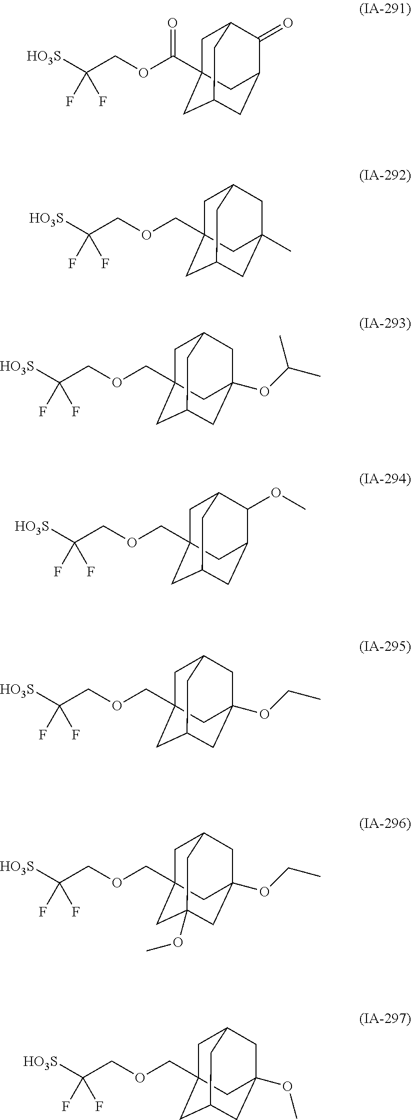

Examples of the acid represented by the formula (IID) include the followings.

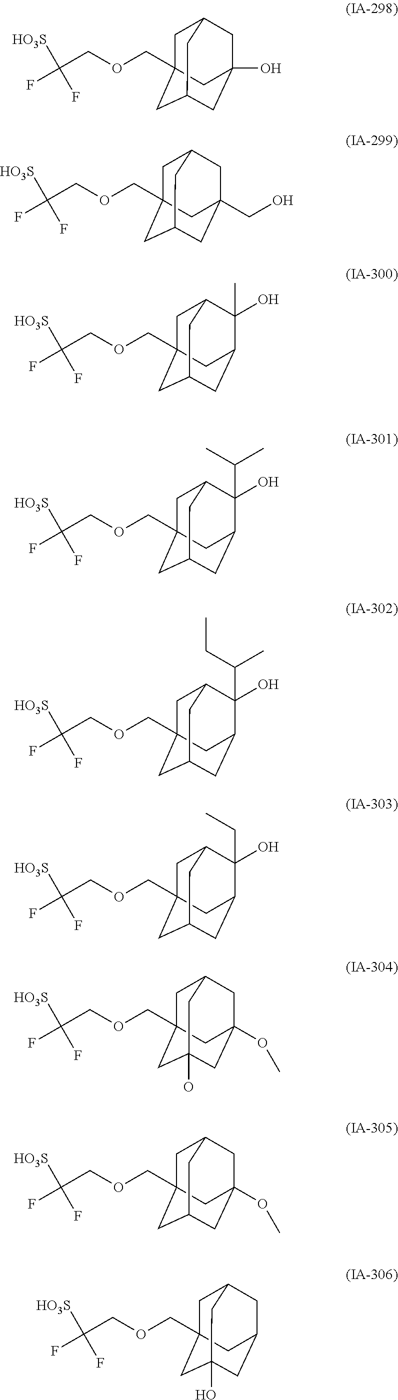

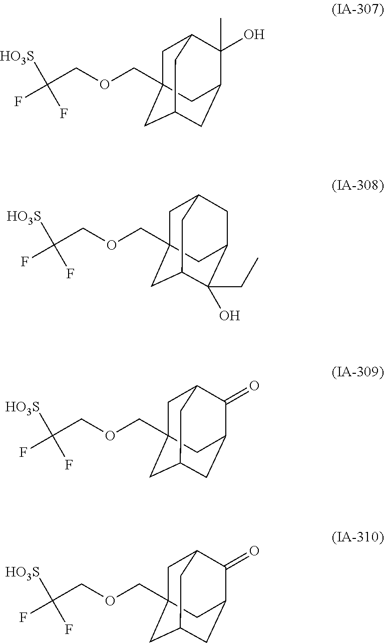

Compound (D) is a compound formed by combining any one of the above-mentioned cations represented by the formulae (ID-1) to (ID-66) with any one of anions derived from the above-mentioned acids represented by the formulae (IA-1) to (IA-310). Specific examples of Compound (D) are shown in the following Tables. The anions derived from the above-mentioned acids represented by the formulae (IA-1) to (IA-310) are anion wherein —SO₃H in the above-mentioned acids represented by the formulae (IA-1) to (IA-310) are converted to —SO₃⁻. For example, Compound (D-102) is represented by the following formula:

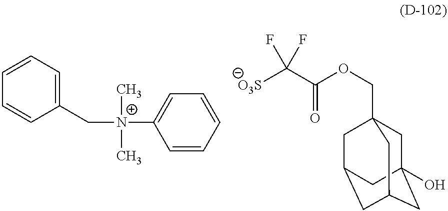

TABLE 1

| | | Acid from which |
|---|---|---|
| Compound (D) | Cation | Anion is derived |
| D-1 | ID-1 | IA-1 |
| D-2 | ID-2 | IA-2 |
| D-3 | ID-3 | IA-3 |
| D-4 | ID-4 | IA-4 |
| D-5 | ID-5 | IA-5 |
| D-6 | ID-8 | IA-6 |
| D-7 | ID-12 | IA-7 |
| D-8 | ID-16 | IA-8 |
| D-9 | ID-21 | IA-9 |
| D-10 | ID-22 | IA-10 |
| D-11 | ID-29 | IA-11 |
| D-12 | ID-30 | IA-12 |
| D-13 | ID-31 | IA-13 |
| D-14 | ID-35 | IA-14 |
| D-15 | ID-37 | IA-15 |
| D-16 | ID-38 | IA-16 |
| D-17 | ID-47 | IA-17 |
| D-18 | ID-49 | IA-18 |
| D-19 | ID-52 | IA-19 |
| D-20 | ID-59 | IA-20 |

TABLE 2

| | | Acid from which |
|---|---|---|
| Compound (D) | Cation | Anion is derived |
| D-21 | ID-60 | IA-21 |
| D-22 | ID-62 | IA-22 |
| D-23 | ID-63 | IA-23 |
| D-24 | ID-64 | IA-24 |
| D-25 | ID-1 | IA-25 |
| D-26 | ID-2 | IA-26 |
| D-27 | ID-3 | IA-27 |
| D-28 | ID-4 | IA-28 |
| D-29 | ID-5 | IA-29 |
| D-30 | ID-8 | IA-30 |
| D-31 | ID-12 | IA-31 |
| D-32 | ID-16 | IA-32 |
| D-33 | ID-1 | IA-33 |
| D-34 | ID-10 | IA-33 |
| D-35 | ID-12 | IA-33 |
| D-36 | ID-21 | IA-33 |
| D-37 | ID-22 | IA-33 |
| D-38 | ID-30 | IA-33 |
| D-39 | ID-47 | IA-33 |
| D-40 | ID-64 | IA-33 |
| D-41 | ID-22 | IA-34 |
| D-42 | ID-29 | IA-35 |
| D-43 | ID-30 | IA-36 |
| D-44 | ID-31 | IA-37 |
| D-45 | ID-35 | IA-38 |
| D-46 | ID-37 | IA-39 |
| D-47 | ID-38 | IA-40 |
| D-48 | ID-47 | IA-41 |
| D-49 | ID-49 | IA-42 |
| D-50 | ID-52 | IA-43 |

TABLE 3

| | | Acid from which |
|---|---|---|
| Compound (D) | Cation | Anion is derived |
| D-51 | ID-59 | IA-44 |
| D-52 | ID-60 | IA-45 |
| D-53 | ID-62 | IA-46 |
| D-54 | ID-63 | IA-47 |
| D-55 | ID-64 | IA-48 |
| D-56 | ID-1 | IA-49 |
| D-57 | ID-2 | IA-50 |
| D-58 | ID-3 | IA-51 |
| D-59 | ID-4 | IA-52 |
| D-60 | ID-5 | IA-53 |
| D-61 | ID-8 | IA-54 |
| D-62 | ID-12 | IA-55 |
| D-63 | ID-16 | IA-56 |
| D-64 | ID-21 | IA-57 |
| D-65 | ID-22 | IA-58 |
| D-66 | ID-29 | IA-59 |
| D-67 | ID-30 | IA-60 |
| D-68 | ID-31 | IA-61 |
| D-69 | ID-35 | IA-62 |
| D-70 | ID-37 | IA-63 |
| D-71 | ID-38 | IA-64 |
| D-72 | ID-47 | IA-65 |
| D-73 | ID-49 | IA-66 |
| D-74 | ID-52 | IA-67 |
| D-75 | ID-59 | IA-68 |
| D-76 | ID-60 | IA-69 |
| D-77 | ID-62 | IA-70 |
| D-78 | ID-63 | IA-71 |
| D-79 | ID-64 | IA-72 |
| D-80 | ID-1 | IA-73 |

TABLE 4

| | | Acid from which |
|---|---|---|
| Compound (D) | Cation | Anion is derived |
| D-81 | ID-2 | IA-74 |
| D-82 | ID-3 | IA-75 |
| D-83 | ID-4 | IA-76 |
| D-84 | ID-5 | IA-77 |
| D-85 | ID-8 | IA-78 |
| D-86 | ID-12 | IA-79 |
| D-87 | ID-16 | IA-80 |
| D-88 | ID-21 | IA-81 |
| D-89 | ID-22 | IA-82 |
| D-90 | ID-29 | IA-83 |
| D-91 | ID-30 | IA-84 |
| D-92 | ID-31 | IA-85 |
| D-93 | ID-35 | IA-86 |
| D-94 | ID-37 | IA-87 |
| D-95 | ID-38 | IA-88 |
| D-96 | ID-47 | IA-89 |
| D-97 | ID-49 | IA-90 |
| D-98 | ID-52 | IA-91 |
| D-99 | ID-59 | IA-92 |
| D-100 | ID-60 | IA-93 |
| D-101 | ID-1 | IA-94 |
| D-102 | ID-10 | IA-94 |
| D-103 | ID-12 | IA-94 |
| D-104 | ID-21 | IA-94 |
| D-105 | ID-22 | IA-94 |
| D-106 | ID-30 | IA-94 |
| D-107 | ID-64 | IA-94 |
| D-108 | ID-62 | IA-95 |
| D-109 | ID-64 | IA-96 |
| D-110 | ID-1 | IA-97 |

TABLE 5

| | | Acid from which |
|---|---|---|
| Compound (D) | Cation | Anion is derived |
| D-111 | ID-2 | IA-98 |
| D-112 | ID-3 | IA-99 |
| D-113 | ID-4 | IA-100 |
| D-114 | ID-5 | IA-101 |
| D-115 | ID-8 | IA-102 |
| D-116 | ID-12 | IA-103 |
| D-117 | ID-16 | IA-104 |
| D-118 | ID-21 | IA-105 |
| D-119 | ID-22 | IA-106 |
| D-120 | ID-29 | IA-107 |
| D-121 | ID-30 | IA-108 |
| D-122 | ID-31 | IA-109 |
| D-123 | ID-35 | IA-110 |
| D-124 | ID-37 | IA-111 |
| D-125 | ID-38 | IA-112 |
| D-126 | ID-47 | IA-113 |
| D-127 | ID-49 | IA-114 |
| D-128 | ID-52 | IA-115 |
| D-129 | ID-59 | IA-116 |
| D-130 | ID-60 | IA-117 |
| D-131 | ID-62 | IA-118 |
| D-132 | ID-63 | IA-119 |
| D-133 | ID-64 | IA-120 |
| D-134 | ID-1 | IA-121 |
| D-135 | ID-2 | IA-122 |
| D-136 | ID-3 | IA-123 |
| D-137 | ID-4 | IA-124 |
| D-138 | ID-5 | IA-125 |
| D-139 | ID-8 | IA-126 |
| D-140 | ID-12 | IA-127 |

TABLE 6

| | | Acid from which |
|---|---|---|
| Compound (D) | Cation | Anion is derived |
| D-141 | ID-16 | IA-128 |
| D-142 | ID-21 | IA-129 |
| D-143 | ID-22 | IA-130 |
| D-144 | ID-29 | IA-131 |
| D-145 | ID-30 | IA-132 |
| D-146 | ID-31 | IA-133 |
| D-147 | ID-1 | IA-134 |
| D-148 | ID-10 | IA-134 |
| D-149 | ID-12 | IA-134 |
| D-150 | ID-21 | IA-134 |
| D-151 | ID-22 | IA-134 |
| D-152 | ID-30 | IA-134 |
| D-153 | ID-47 | IA-134 |
| D-154 | ID-64 | IA-134 |
| D-155 | ID-37 | IA-135 |
| D-156 | ID-38 | IA-136 |
| D-157 | ID-47 | IA-137 |
| D-158 | ID-49 | IA-138 |
| D-159 | ID-52 | IA-139 |
| D-160 | ID-59 | IA-140 |
| D-161 | ID-60 | IA-141 |
| D-162 | ID-62 | IA-142 |
| D-163 | ID-63 | IA-143 |
| D-164 | ID-64 | IA-144 |
| D-165 | ID-1 | IA-145 |
| D-166 | ID-2 | IA-146 |
| D-167 | ID-3 | IA-147 |
| D-168 | ID-4 | IA-148 |
| D-169 | ID-5 | IA-149 |
| D-170 | ID-8 | IA-150 |

TABLE 7

| | | Acid from which |
|---|---|---|
| Compound (D) | Cation | Anion is derived |
| D-171 | ID-12 | IA-151 |
| D-172 | ID-16 | IA-152 |
| D-173 | ID-21 | IA-153 |
| D-174 | ID-22 | IA-154 |
| D-175 | ID-29 | IA-155 |
| D-176 | ID-30 | IA-156 |
| D-177 | ID-31 | IA-157 |
| D-178 | ID-35 | IA-158 |
| D-179 | ID-37 | IA-159 |
| D-180 | ID-38 | IA-160 |
| D-181 | ID-47 | IA-161 |
| D-182 | ID-49 | IA-162 |
| D-183 | ID-1 | IA-163 |
| D-184 | ID-10 | IA-163 |
| D-185 | ID-12 | IA-163 |
| D-186 | ID-21 | IA-163 |
| D-187 | ID-22 | IA-163 |
| D-188 | ID-30 | IA-163 |
| D-189 | ID-47 | IA-163 |
| D-190 | ID-64 | IA-163 |
| D-191 | ID-5 | IA-164 |
| D-192 | ID-8 | IA-165 |
| D-193 | ID-12 | IA-166 |
| D-194 | ID-16 | IA-167 |
| D-195 | ID-21 | IA-168 |
| D-196 | ID-22 | IA-169 |
| D-197 | ID-29 | IA-170 |
| D-198 | ID-30 | IA-171 |
| D-199 | ID-31 | IA-172 |
| D-200 | ID-35 | IA-173 |

TABLE 8

| | | Acid from which |
|---|---|---|
| Compound (D) | Cation | Anion is derived |
| D-201 | ID-37 | IA-174 |
| D-202 | ID-38 | IA-175 |
| D-203 | ID-47 | IA-176 |
| D-204 | ID-49 | IA-177 |
| D-205 | ID-52 | IA-178 |
| D-206 | ID-59 | IA-179 |
| D-207 | ID-60 | IA-180 |
| D-208 | ID-62 | IA-181 |
| D-209 | ID-63 | IA-182 |
| D-210 | ID-64 | IA-183 |
| D-211 | ID-1 | IA-184 |
| D-212 | ID-2 | IA-185 |
| D-213 | ID-3 | IA-186 |
| D-214 | ID-4 | IA-187 |
| D-215 | ID-5 | IA-188 |
| D-216 | ID-8 | IA-189 |
| D-217 | ID-12 | IA-190 |
| D-218 | ID-16 | IA-191 |
| D-219 | ID-21 | IA-192 |
| D-220 | ID-22 | IA-193 |
| D-221 | ID-29 | IA-194 |
| D-222 | ID-30 | IA-195 |
| D-223 | ID-31 | IA-196 |
| D-224 | ID-35 | IA-197 |
| D-225 | ID-37 | IA-198 |
| D-226 | ID-38 | IA-199 |
| D-227 | ID-47 | IA-200 |
| D-228 | ID-49 | IA-201 |
| D-229 | ID-52 | IA-202 |
| D-230 | ID-59 | IA-203 |

TABLE 9

| | | Acid from which |
|---|---|---|
| Compound (D) | Cation | Anion is derived |
| D-231 | ID-60 | IA-204 |
| D-232 | ID-62 | IA-205 |
| D-233 | ID-63 | IA-206 |
| D-234 | ID-64 | IA-207 |
| D-235 | ID-1 | IA-208 |
| D-236 | ID-2 | IA-209 |
| D-237 | ID-3 | IA-210 |
| D-238 | ID-4 | IA-211 |
| D-239 | ID-5 | IA-212 |
| D-240 | ID-8 | IA-213 |
| D-241 | ID-12 | IA-214 |
| D-242 | ID-16 | IA-215 |
| D-243 | ID-21 | IA-216 |
| D-244 | ID-22 | IA-217 |
| D-245 | ID-29 | IA-218 |
| D-246 | ID-30 | IA-219 |
| D-247 | ID-31 | IA-220 |
| D-248 | ID-35 | IA-221 |
| D-249 | ID-37 | IA-222 |
| D-250 | ID-38 | IA-223 |
| D-251 | ID-47 | IA-224 |
| D-252 | ID-49 | IA-225 |
| D-253 | ID-52 | IA-226 |
| D-254 | ID-59 | IA-227 |
| D-255 | ID-60 | IA-228 |
| D-256 | ID-62 | IA-229 |
| D-257 | ID-63 | IA-230 |
| D-258 | ID-64 | IA-231 |
| D-259 | ID-1 | IA-232 |
| D-260 | ID-2 | IA-233 |

TABLE 10

| | | Acid from which |
|---|---|---|
| Compound (D) | Cation | Anion is derived |
| D-261 | ID-3 | IA-234 |
| D-262 | ID-4 | IA-235 |
| D-263 | ID-5 | IA-236 |
| D-264 | ID-8 | IA-237 |
| D-265 | ID-12 | IA-238 |
| D-266 | ID-16 | IA-239 |
| D-267 | ID-21 | IA-240 |
| D-268 | ID-22 | IA-241 |
| D-269 | ID-29 | IA-242 |
| D-270 | ID-30 | IA-243 |
| D-271 | ID-31 | IA-244 |
| D-272 | ID-35 | IA-245 |
| D-273 | ID-37 | IA-246 |
| D-274 | ID-38 | IA-247 |
| D-275 | ID-47 | IA-248 |
| D-276 | ID-49 | IA-249 |
| D-277 | ID-52 | IA-250 |
| D-278 | ID-59 | IA-251 |
| D-279 | ID-60 | IA-252 |
| D-280 | ID-62 | IA-253 |
| D-281 | ID-63 | IA-254 |
| D-282 | ID-64 | IA-255 |
| D-283 | ID-1 | IA-256 |
| D-284 | ID-2 | IA-257 |
| D-285 | ID-3 | IA-258 |
| D-286 | ID-4 | IA-259 |
| D-287 | ID-5 | IA-260 |
| D-288 | ID-8 | IA-261 |
| D-289 | ID-12 | IA-262 |
| D-290 | ID-16 | IA-263 |

TABLE 11

| | | Acid from which |
|---|---|---|
| Compound (D) | Cation | Anion is derived |
| D-291 | ID-21 | IA-264 |
| D-292 | ID-22 | IA-265 |
| D-293 | ID-29 | IA-266 |
| D-294 | ID-30 | IA-267 |
| D-295 | ID-31 | IA-268 |
| D-296 | ID-35 | IA-269 |
| D-297 | ID-37 | IA-270 |
| D-298 | ID-6 | IA-271 |
| D-299 | ID-7 | IA-272 |
| D-300 | ID-9 | IA-273 |
| D-301 | ID-11 | IA-274 |
| D-302 | ID-13 | IA-275 |
| D-303 | ID-14 | IA-246 |
| D-304 | ID-15 | IA-277 |
| D-305 | ID-17 | IA-278 |
| D-306 | ID-18 | IA-279 |
| D-307 | ID-19 | IA-280 |
| D-308 | ID-20 | IA-281 |
| D-309 | ID-23 | IA-282 |
| D-310 | ID-24 | IA-283 |
| D-311 | ID-25 | IA-284 |
| D-312 | ID-26 | IA-285 |
| D-313 | ID-27 | IA-286 |
| D-314 | ID-28 | IA-287 |
| D-315 | ID-32 | IA-288 |
| D-316 | ID-33 | IA-289 |
| D-317 | ID-34 | IA-290 |
| D-318 | ID-36 | IA-291 |
| D-319 | ID-38 | IA-292 |
| D-320 | ID-39 | IA-293 |

TABLE 12

| | | Acid from which |
|---|---|---|
| Compound (D) | Cation | Anion is derived |
| D-321 | ID-40 | IA-294 |
| D-322 | ID-41 | IA-295 |
| D-323 | ID-42 | IA-296 |
| D-324 | ID-43 | IA-297 |
| D-325 | ID-44 | IA-298 |
| D-326 | ID-45 | IA-299 |
| D-327 | ID-46 | IA-300 |
| D-328 | ID-48 | IA-301 |
| D-329 | ID-50 | IA-302 |
| D-330 | ID-51 | IA-303 |
| D-331 | ID-52 | IA-304 |
| D-332 | ID-53 | IA-305 |
| D-333 | ID-54 | IA-306 |
| D-334 | ID-55 | IA-307 |
| D-335 | ID-56 | IA-308 |
| D-336 | ID-57 | IA-309 |
| D-337 | ID-58 | IA-310 |
| D-338 | ID-61 | IA-311 |
| D-339 | ID-65 | IA-312 |
| D-340 | ID-66 | IA-313 |
| D-341 | ID-67 | IA-3 |
| D-342 | ID-67 | IA-33 |
| D-343 | ID-67 | IA-94 |
| D-344 | ID-67 | IA-134 |
| D-345 | ID-67 | IA-163 |

Among them, preferred are Compound (D-34), Compound (D-38), Compound (D-102), Compound (D-106), Compound (D-148), Compound (D-152), Compound (D-184) and Compound (D-188).

The photoresist composition of the present invention can contain two or more kinds of Compound (D). The content of Compound (D) is usually 0.1 to 5% by weight based on amount of solid component. In this specification, “solid component” means components other than solvent in the photoresist composition. The content of Compound (D) and the content of solid component can analyzed with a conventional analysis such as liquid chromatography or gas chromatography.

Next, the resin will be illustrated.

The resin has an acid-labile group and is insoluble or poorly soluble in an alkali aqueous solution but becomes soluble in an alkali aqueous solution by the action of an acid. The resin has a structural unit derived from a compound having an acid-labile group, and can be produced by polymerizing one or more compounds having an acid-labile group.

In this specification, “an acid-labile group” means a group capable of being eliminated by the action of an acid.

Examples of the acid-labile group include a group represented by the formula (1):

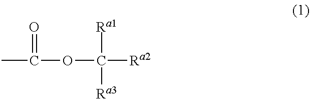

wherein R^{a1}, R^{a2} and R^{a3} independently each represent an aliphatic hydrocarbon group or a saturated cyclic hydrocarbon group, or R^{a1} and R^{a2} are bonded each other to form a ring together with a carbon atom to which R^{a1} and R^{a2} are bonded.

Examples of the aliphatic hydrocarbon group include a C1-C8 alkyl group. Specific examples of the C1-C8 alkyl group include a methyl group, an ethyl group, a propyl group, an isopropyl group, a butyl group, a pentyl group, a hexyl group, a heptyl group and an octyl group. The saturated cyclic hydrocarbon group may be monocyclic or polycyclic, and examples thereof include a monocyclic alicyclic hydrocarbon group such as a C3-C20 cycloalkyl group (e.g. a cyclopentyl group, a cyclohexyl group, a methylcyclohexyl group, a dimethylcyclohexyl group, a cycloheptyl group and a cyclooctyl group) and a polycyclic alicyclic hydrocarbon group such as a decahydronaphthyl group, an adamantyl group, a norbornyl group, a methylnorbornyl group, and the followings:

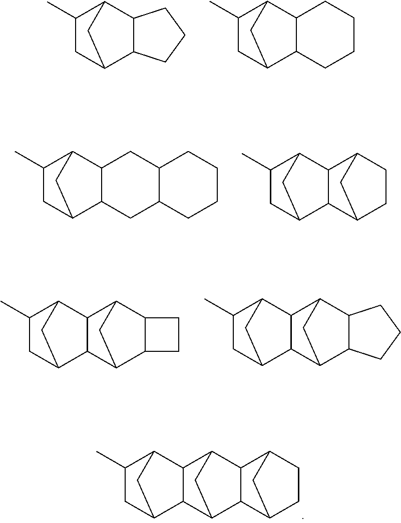

The saturated cyclic hydrocarbon group preferably has 3 to 20 carbon atoms.

Examples of the ring formed by bonding R^{a1} and R^{a2} each other include the following groups and the ring preferably has 3 to 20 carbon atoms, and the more preferably has 3 to 12 carbon atoms.

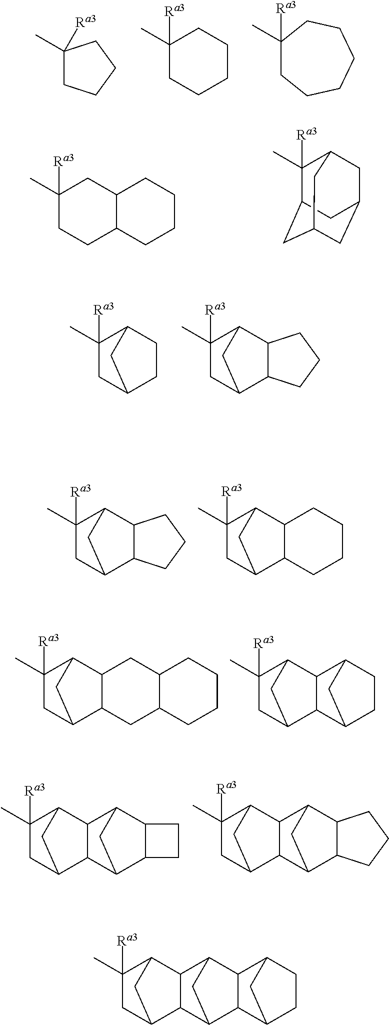

wherein R^{a3} is the same as defined above.

The group represented by the formula (1) wherein R^{a1}, R^{a2} and R^{a3} independently each represent a C1-C8 alkyl group such as a tert-butyl group, the group represented by the formula (1) wherein R^{a1} and R^{a2} are bonded each other to form an adamantyl ring and R^{a3} is a C1-C8 alkyl group such as a 2-alkyl-2-adamantyl group, and the group represented by the formula (1) wherein R^{a1} and R^{a2} are C1-C8 alkyl groups and R^{a3} is an adamantyl group such as a 1-(1-adamantyl)-1-alkylalkoxycarbonyl group are preferable.

The compound having an acid-labile group is preferably an acrylate monomer having an acid-labile group in its side chain or a methacryalte monomer having an acid-labile group in its side chain.

Preferable examples of the compound having an acid-labile group include monomers represented by the formulae (a1-1) and (a1-2):

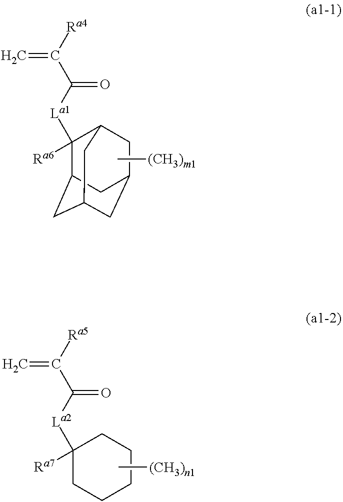

wherein R^{a4} and R^{a5} each independently represents a hydrogen atom or a methyl group, R^{a6} and R^{a7} each independently represents a C1-C8 aliphatic hydrocarbon group or a C3-C10 saturated cyclic hydrocarbon group, L^{a1} and L^{a2} each independently represents *—O— or *—O—(CH₂)ₖ₁—CO—O— in which * represents a binding position to —CO—, and k1 represents an integer of 1 to 7, m1 represents an integer of 0 to 14 and n1 represents an integer of 0 to 10.

The aliphatic hydrocarbon group preferably has 1 to 6 carbon atoms, and the saturated cyclic hydrocarbon group preferably has 3 to 8 carbon atoms and more preferably 3 to 6 carbon atoms.

Examples of the aliphatic hydrocarbon group include a C1-C8 alkyl group such as a methyl group, an ethyl group, a propyl group, an isopropyl group, a butyl group, a tert-butyl group, a 2,2-dimethylethyl group, a 1-methylpropyl group, a 2,2-dimethylpropyl group, a 1-ethylpropyl group, a 1-methylbutyl group, a 2-methylbutyl group, a 3-methylbutyl group, a 1-propylbutyl group, a pentyl group, a 1-methylpentyl group, a hexyl group, a 1,4-dimethylhexyl group, a heptyl group, a 1-methylheptyl group and an octyl group. Examples of the saturated cyclic hydrocarbon group include a cyclohexyl group, a methylcyclohexyl group, a dimethylcyclohexyl group, a cycloheptyl group, a methylcycloheptyl group, a norbornyl group and a methylnorbornyl group. R^{a4} is preferably a methyl group, an ethyl group or an isopropyl group, and R^{a5} is preferably a methyl group, an ethyl group or an isopropyl group.

L^{a1} is preferably *—O— or *—O—(CH₂)_{f1}—CO—O— in which * represents a binding position to —CO—, and f1 represents an integer of 1 to 4, and is more preferably *—O— or *—O—CH₂—CO—O—, and is especially preferably *—O—. L^{a2} is preferably *—O— or *—O—(CH₂)_{f1}—CO—O— in which * represents a binding position to —CO—, and f1 is the same as defined above, and is more preferably *—O— or *—O—CH₂—CO—O—, and is especially preferably *—O—.

In the formula (a1-1), m1 is preferably an integer of 0 to 3, and is more preferably 0 or 1. In the formula (a1-2), n1 is preferably an integer of 0 to 3, and is more preferably 0 or 1.

Particularly when the photoresist composition contains a resin derived from a monomer having a bulky structure such as a saturated cyclic hydrocarbon group, the photoresist composition having excellent resolution tends to be obtained.

Examples of the monomer represented by the formula (a1-1) include the followings.

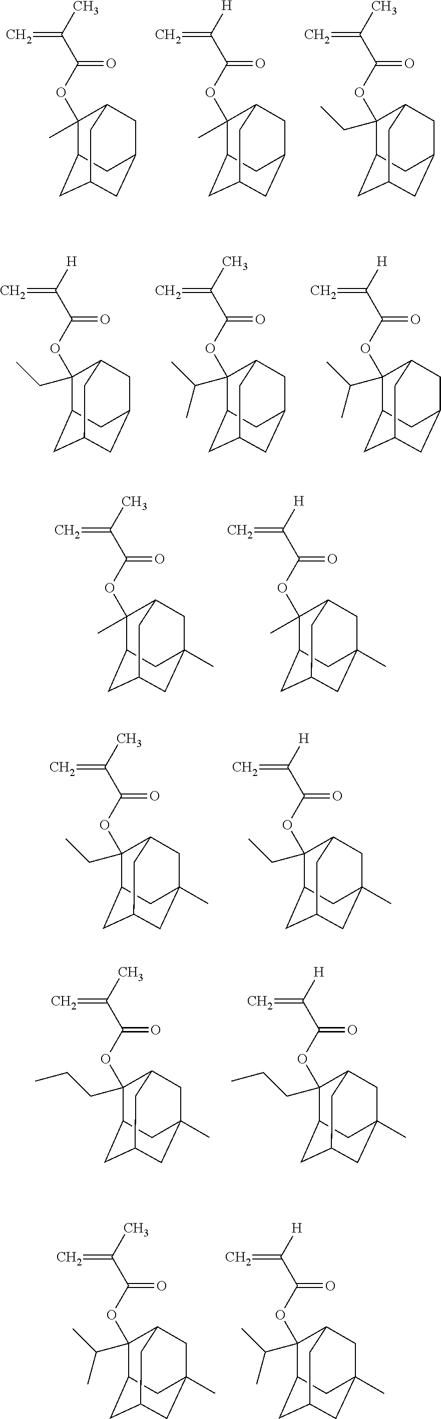

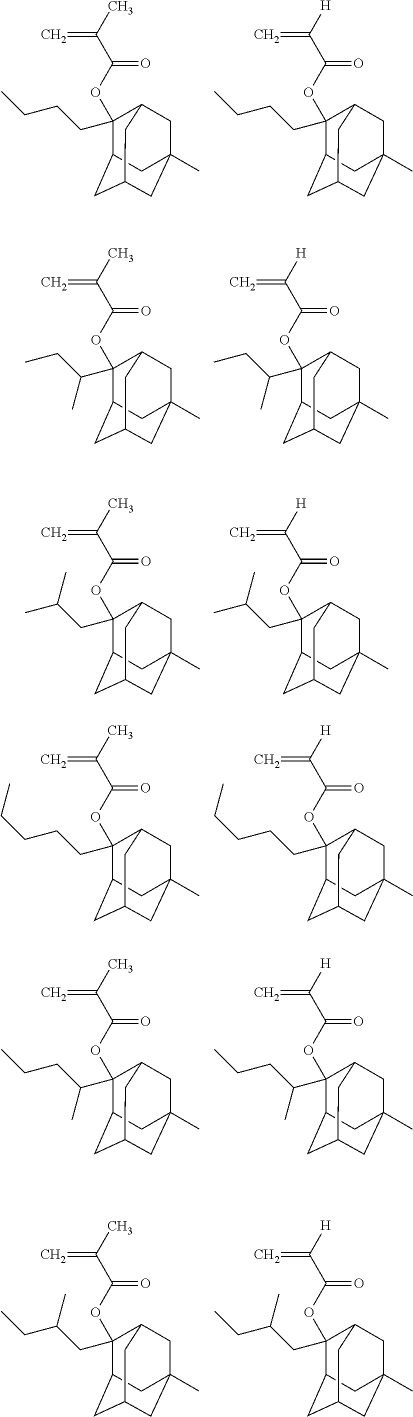

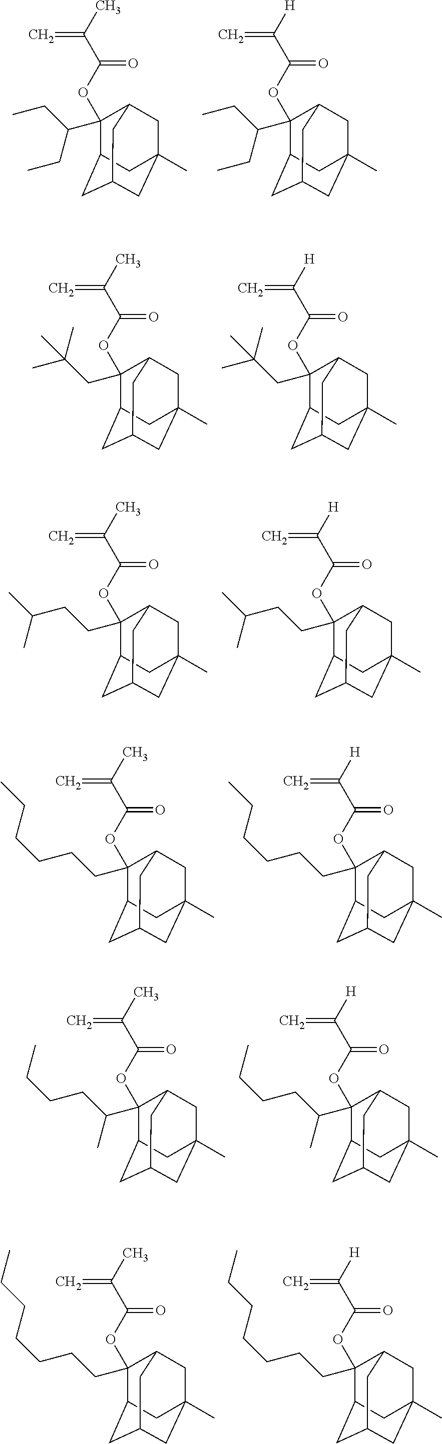

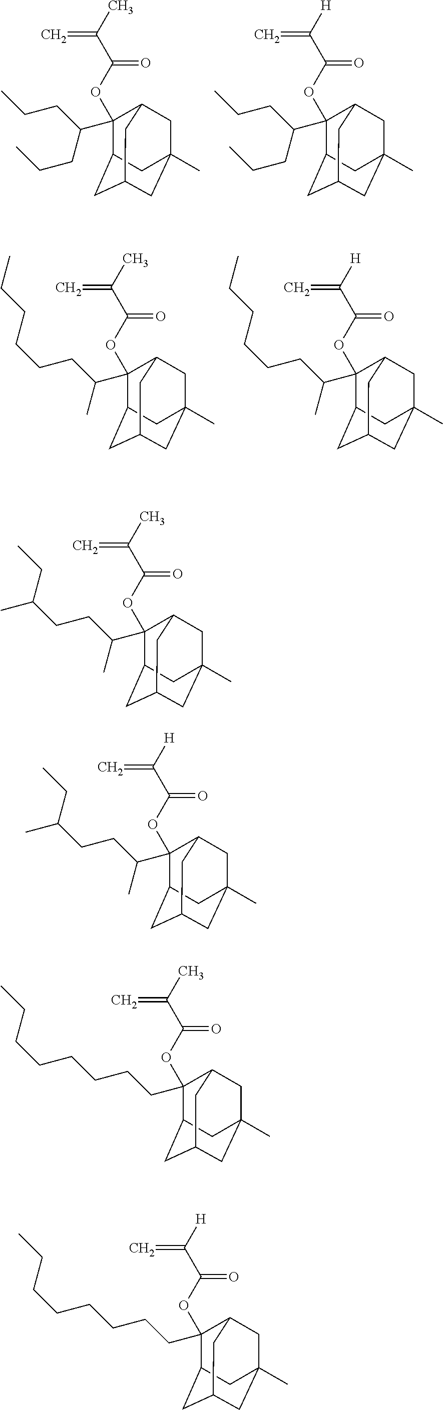

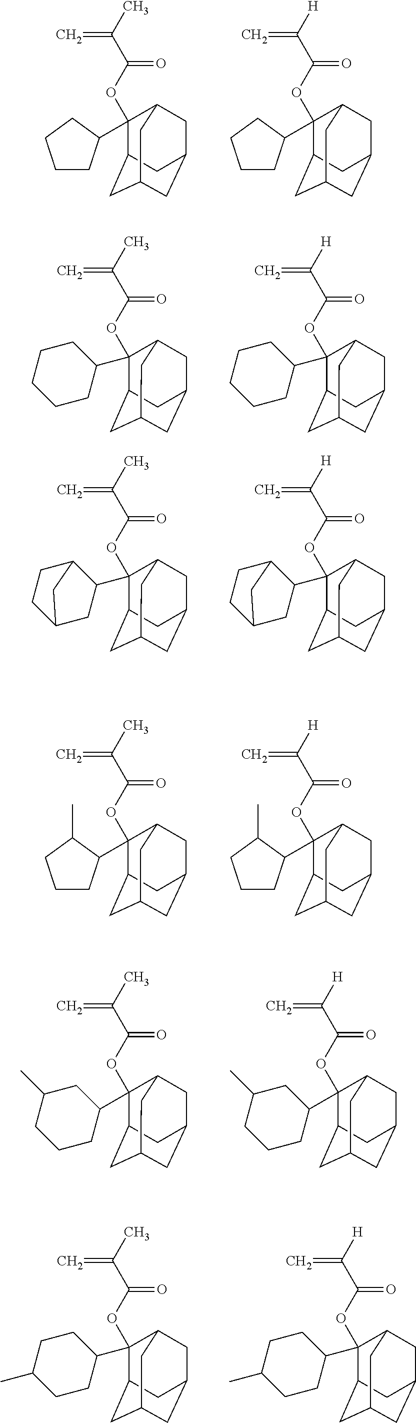

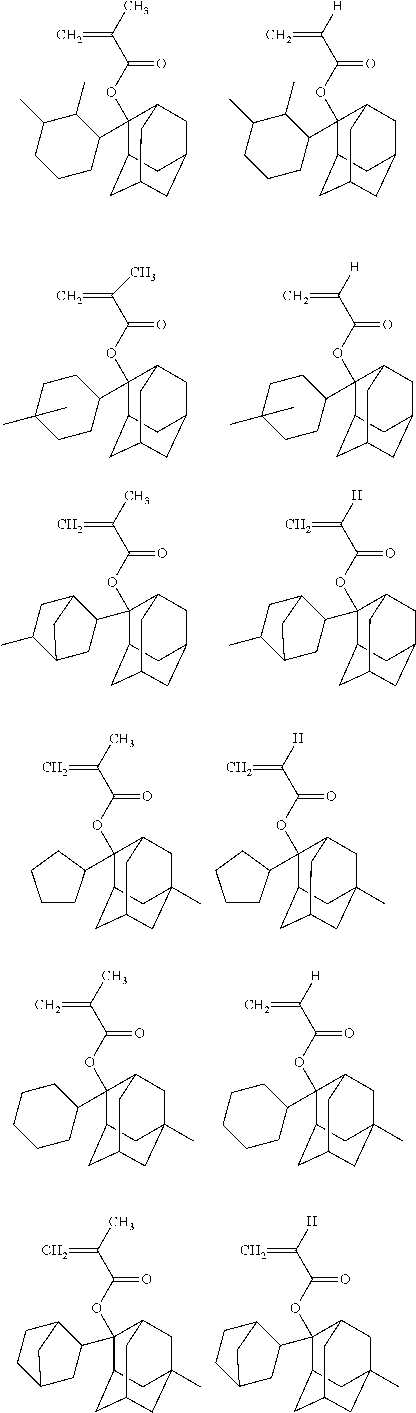

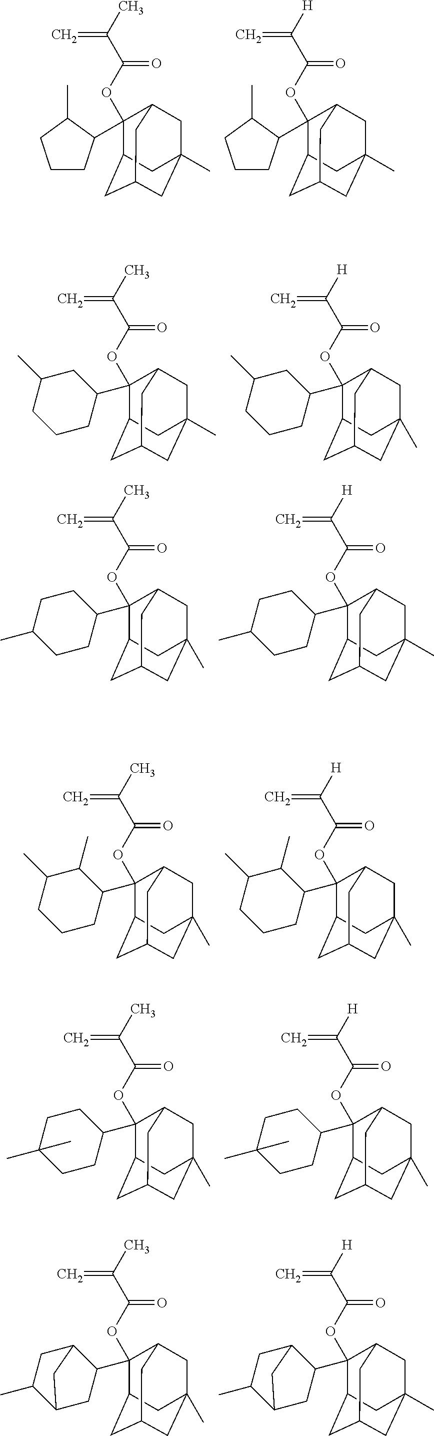

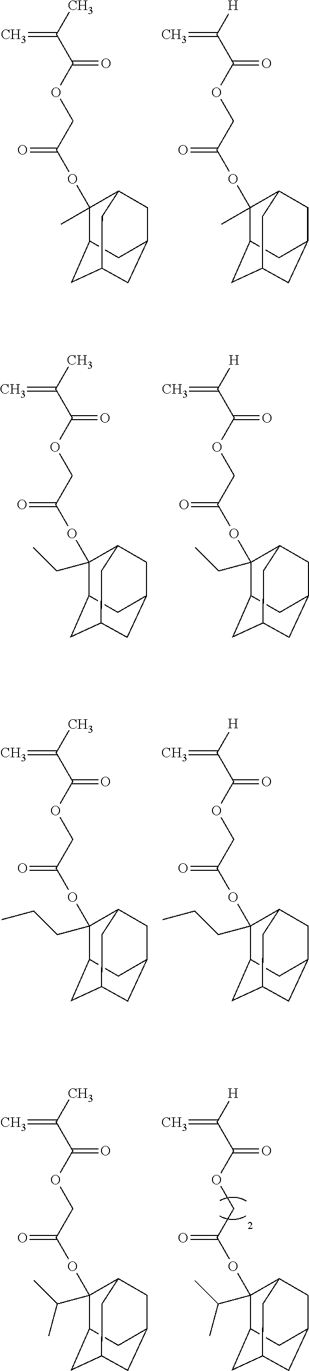

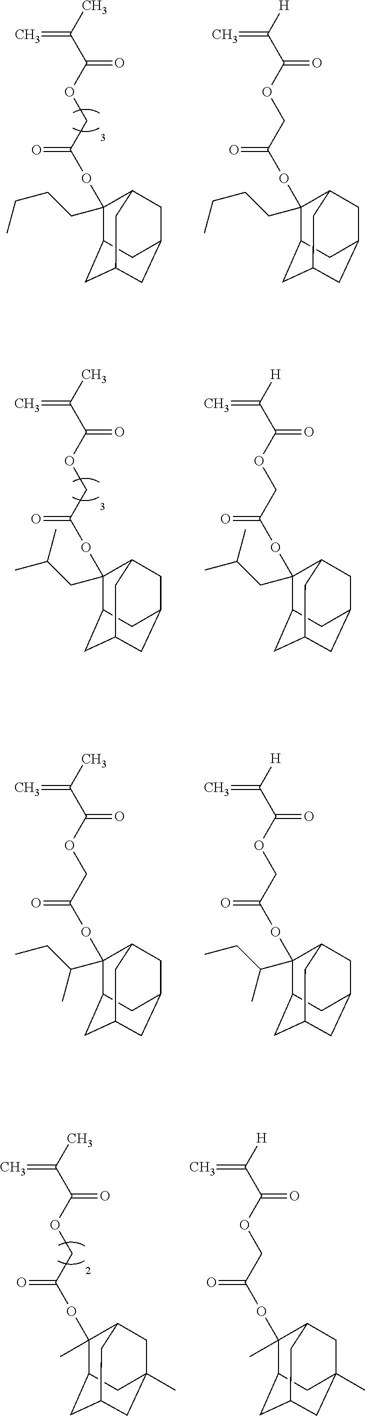

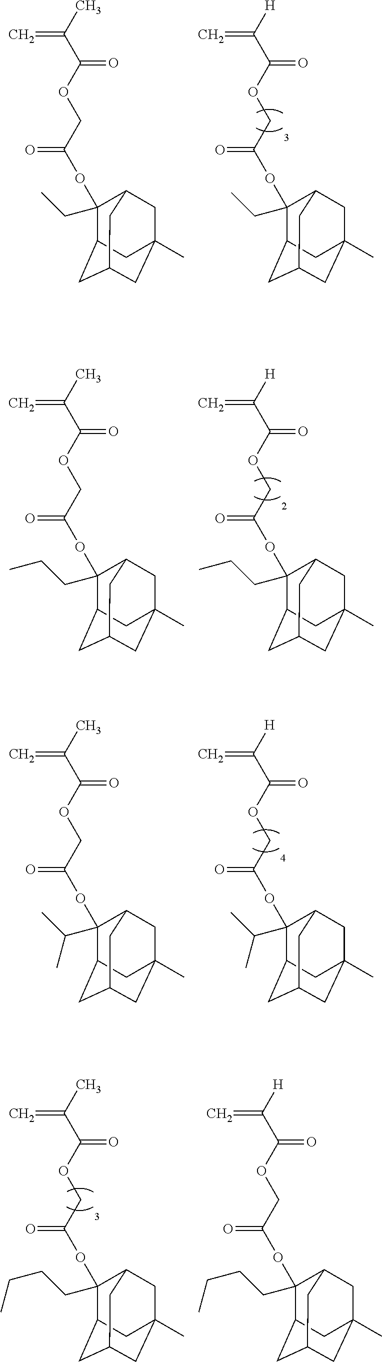

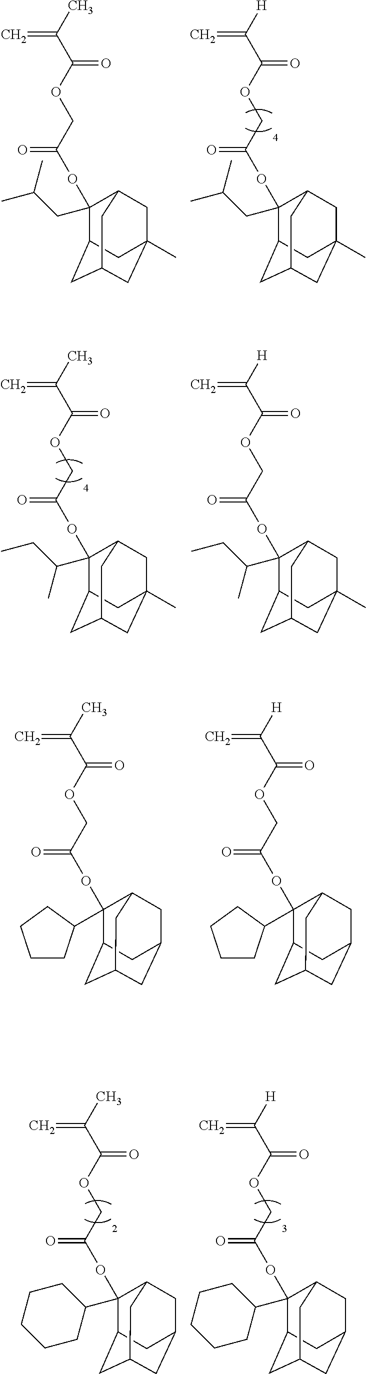

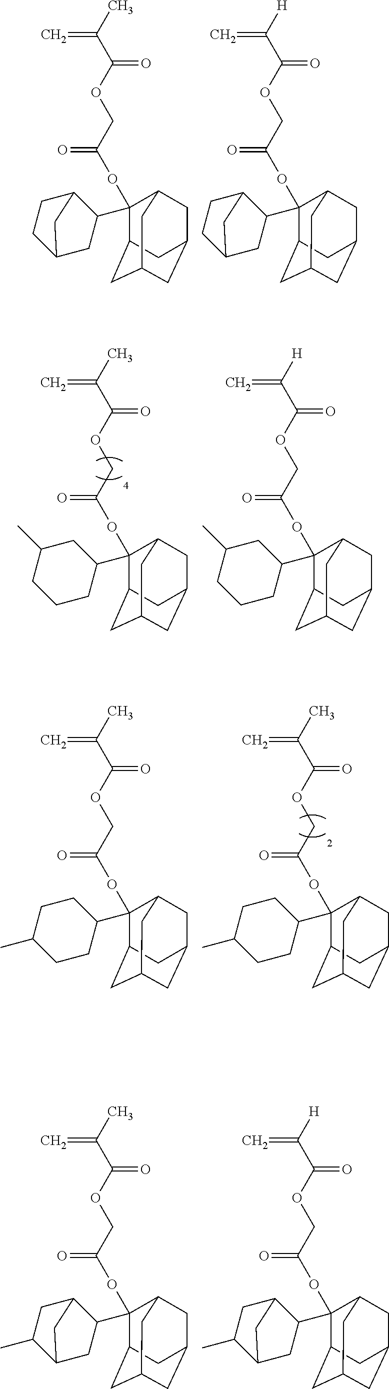

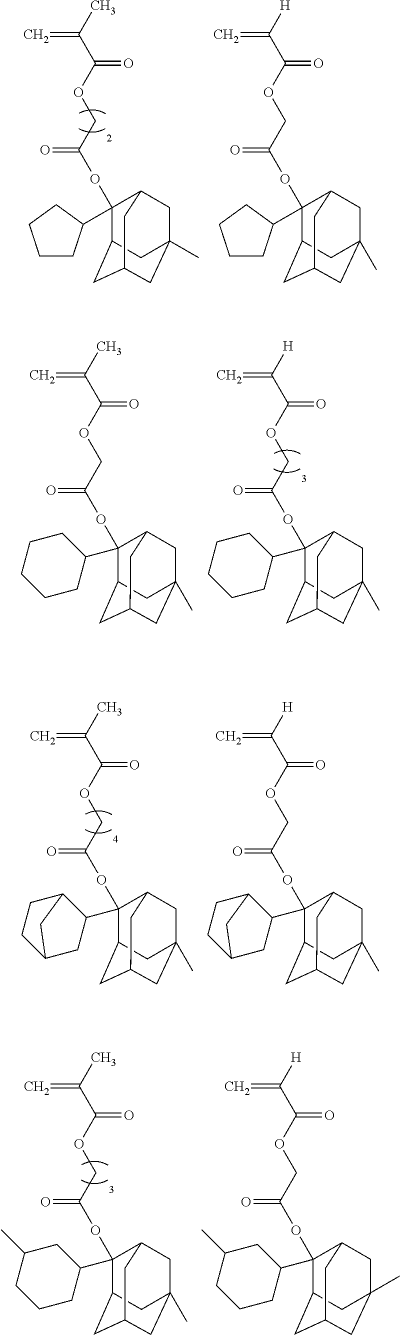

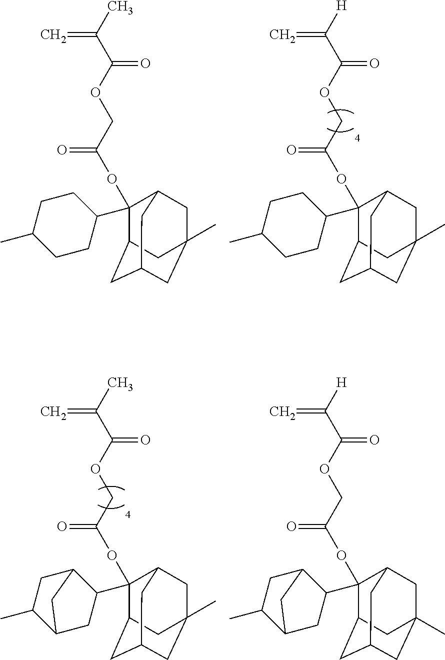

Among them, preferred are 2-methyl-2-adamantyl acrylate, 2-methyl-2-adamantyl methacrylate, 2-ethyl-2-adamantyl acrylate, 2-ethyl-2-adamantyl methacrylate, 2-isopropyl-2-adamantyl acrylate and 2-isopropyl-2-adamantyl methacrylate, and more preferred are 2-methyl-2-adamantylmethacrylate, 2-ethyl-2-adamantylmethacrylate, and 2-isopropyl-2-adamantyl methacrylate.

Examples of the monomer represented by the formula (a1-2) include the followings.

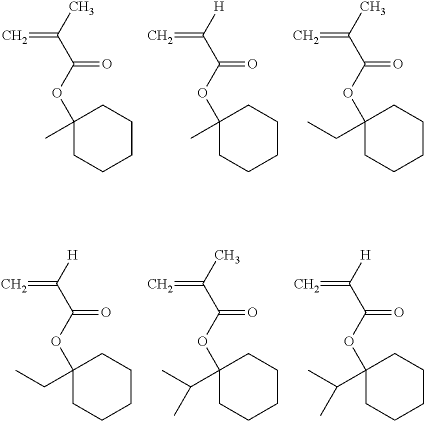

Among them, preferred are 1-ethyl-1-cyclohexyl acrylate and 1-ethyl-1-cyclohexyl methacrylate, and more preferred is 1-ethyl-1-cyclohexyl methacrylate.

The content of the structural unit derived from a compound having an acid-labile group in the resin is usually 10 to 95% by mole, preferably 15 to 90% by mole and more preferably 20 to 85% by mole based on 100% by mole of all the structural units of the resin.

Other examples of the compound having an acid-labile group include a monomer represented by the formula (a1-3):

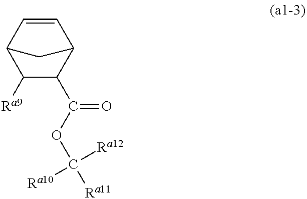

wherein R^{a9} represents a hydrogen atom, a C1-C3 aliphatic hydrocarbon group which can have one or more substituents, a carboxyl group, a cyano group or a —COOR^{a13} group in which R^{a13} represents a C1-C8 aliphatic hydrocarbon group or a C3-C8 saturated cyclic hydrocarbon group, and the C1-C8 aliphatic hydrocarbon group and the C3-C8 saturated cyclic hydrocarbon group can have one or more hydroxyl groups, and one or more —CH₂— in the C1-C8 aliphatic hydrocarbon group and the C3-C8 saturated cyclic hydrocarbon group can be replaced by —O— or —CO—, R^{a10}, R^{a11} and R^{a12} each independently represent a C1-C12 aliphatic hydrocarbon group or a C3-C12 saturated cyclic hydrocarbon group, and R^{a10} and R^{a11} can be bonded each other to form a ring together with the carbon atom to which R^{a10} and R^{a11} are bonded, and the C1-C12 aliphatic hydrocarbon group and the C3-C12 saturated cyclic hydrocarbon group can have one or more hydroxyl groups, and one or more —CH₂— in the C1-C12 aliphatic hydrocarbon group and the C3-C12 saturated cyclic hydrocarbon group can be replaced by —O— or —CO—.

Examples of the substituent include a hydroxyl group. Examples of the C1-C3 aliphatic hydrocarbon group which can have one or more substituents include a methyl group, an ethyl group, a propyl group, a hydroxymethyl group and a 2-hydroxyethyl group. Examples of R^{a13} include a methyl group, an ethyl group, a propyl group, a 2-oxo-oxolan-3-yl group and a 2-oxo-oxolan-4-yl group. Examples of R^{a10}, R^{a11} and R^{a12} include a methyl group, an ethyl group, a cyclohexyl group, a methylcyclohexyl group, a hydroxycyclohexyl group, an oxocyclohexyl group and an adamantyl group, and examples of the ring formed by bonding R^{a10} and R^{a11} each other together with the carbon atom to which R^{a10} and R^{a11} are bonded include a cyclohexane ring and an adamantane ring.

Examples of the monomer represented by the formula (a1-3) include tert-butyl 5-norbornene-2-carboxylate,
1-cyclohexyl-1-methylethyl 5-norbornene-2-carboxylate,
1-methylcyclohexyl 5-norbornene-2-carboxylate,
2-methyl-2-adamantyl 5-norbornene-2-carboxylate,
2-ethyl-2-adamantyl 5-norbornene-2-carboxylate,
1-(4-methylcyclohexyl)-1-methylethyl 5-norbornene-2-carboxylate,
1-(4-hydroxylcyclohexyl)-1-methylethyl 5-norbornene-2-carboxylate,
1-methyl-1-(4-oxocyclohexyl)ethyl 5-norbornene-2-carboxylate and
1-(1-adamantyl)-1-methylethyl 5-norbornene-2-carboxylate.

When the resin has a structural unit derived from the monomer represented by the formula (a1-3), the photoresist composition having excellent resolution and higher dry-etching resistance tends to be obtained.

When the resin contains the structural unit derived form the monomer represented by the formula (a1-3), the content of the structural unit derived from the monomer represented by the formula (a1-3) is usually 10 to 95% by mole and preferably 15 to 90% by mole and more preferably 20 to 85% by mole based on total molar of all the structural units of the resin.

Other examples of the compound having an acid-labile group include a monomer represented by the formula (a1-4):

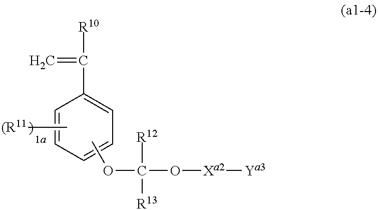

wherein R¹⁰ represents a hydrogen atom, a halogen atom, a C1-C6 alkyl group or a C1-C6 halogenated alkyl group, R¹¹ is independently in each occurrence a halogen atom, a hydroxyl group, a C1-C6 alkyl group, a C1-C6 alkoxy group, a C2-C4 acyl group, a C2-C4 acyloxy group, an acryloyl group or a methacryloyl group, 1a represents an integer of 0 to 4, R¹² and R¹³ each independently represent a hydrogen atom or a C1-C12 hydrocarbon group, X^{a2} represents a single bond or a C1-C17 divalent saturated hydrocarbon group in which one or more —CH₂— can be replaced by —O—, —CO—, —S—, —SO₂— or —N(R^{c})— wherein R^{c} represents a hydrogen atom or a C1-C6 alkyl group, and Y^{a3} represents a C1-C12 aliphatic hydrocarbon group, a C3-C18 saturated cyclic hydrocarbon group or a C6-C18 aromatic hydrocarbon group, and the C1-C12 aliphatic hydrocarbon group, the C2-C18 saturated cyclic hydrocarbon group and the C6-C18 aromatic hydrocarbon group can have one or more substituents.

Examples of the halogen atom include a fluorine atom.

Examples of the C1-C6 alkyl group include a methyl group, an ethyl group, a propyl group, an isopropyl group, a butyl group, an isobutyl group, a sec-butyl group, a tert-butyl group, a pentyl group and a hexyl group, and a C1-C4 alkyl group is preferable and a C1-C2 alkyl group is more preferable and a methyl group is especially preferable.

Examples of the C1-C6 halogenated alkyl group include a trifluoromethyl group, a pentafluoroethyl group, a heptafluoropropyl group, a heptafluoroisopropyl group, a nonafluorobutyl group, a nonafluoro-sec-butyl group, a nonafluoro-tert-butyl group, a perfluoropentyl group and a perfluorohexyl group.

Examples of the C1-C6 alkoxy group include a methoxy group, an ethoxy group, a propoxy group, an isopropoxy group, a butoxy group, an isobutoxy group, a sec-butoxy group, a tert-butoxy group, a pentyloxy group and a hexyloxy group, and a C1-C4 alkoxy group is preferable and a C1-C2 alkoxy group is more preferable and a methoxy group is especially preferable.

Examples of the C2-C4 acyl group include an acetyl group, a propionyl group and a butyryl group, and examples of the C2-C4 acyloxy group include an acetyloxy group, a propionyloxy group and a butyryloxy group.

Examples of the C1-C12 hydrocarbon group include a C1-C12 aliphatic hydrocarbon group such as a methyl group, an ethyl group, a propyl group, an isopropyl group, a butyl group, an isobutyl group, a sec-butyl group, a tert-butyl group, a pentyl group, a hexyl group, a heptyl group, an octyl group, a 2-ethylhexyl group, a nonyl group, a decyl group, an undecyl group and a dodecyl group, and a C3-C12 saturated cyclic hydrocarbon group such as a cyclohexyl group, an adamantyl group, a 2-alkyl-2-adamantyl group, a 1-(1-adamantyl)-1-alkyl group and an isobornyl group.

Examples of the C1-C17 divalent saturated hydrocarbon group include a C1-C17 alkanediyl group such as a methylene group, an ethylene group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, a undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group and a heptadecane-1,17-diyl group.

Examples of the C1-C12 aliphatic hydrocarbon group include a methyl group, an ethyl group, a propyl group, an isopropyl group, a butyl group, an isobutyl group, a sec-butyl group, a tert-butyl group, a pentyl group, a hexyl group, a heptyl group, an octyl group, a 2-ethylhexyl group, a nonyl group, a decyl group, an undecyl group and a dodecyl group. Examples of the C3-C18 saturated cyclic hydrocarbon group include a cyclopropyl group, a cyclobutyl group, a cyclopentyl group, a cyclohexyl group, a cycloheptyl group, a cyclooctyl group, a cyclononyl group, a cyclodecyl group, a norbornyl group, a 1-adamantyl group, a 2-adamantyl group, an isobornyl group and the following groups:

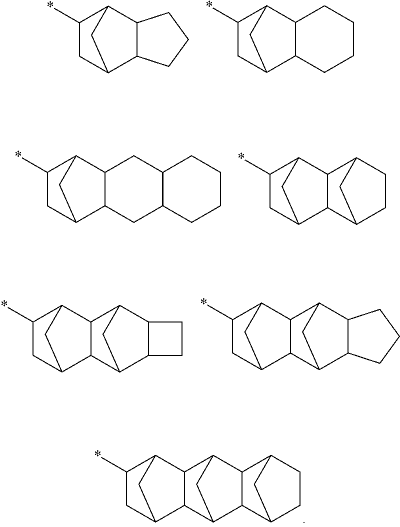

Examples of the C6-C18 aromatic hydrocarbon group include a phenyl group, a naphthyl group, an anthryl group, a p-methylphenyl group, a p-tert-butylphenyl group and a p-adamantylphenyl group.

Examples of the monomer represented by the formula (a1-4) include the followings.

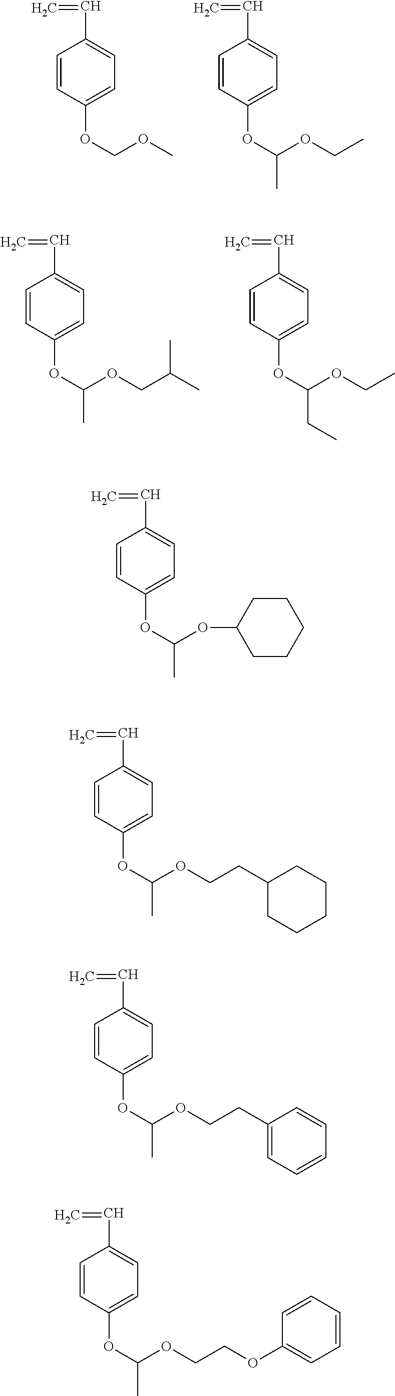

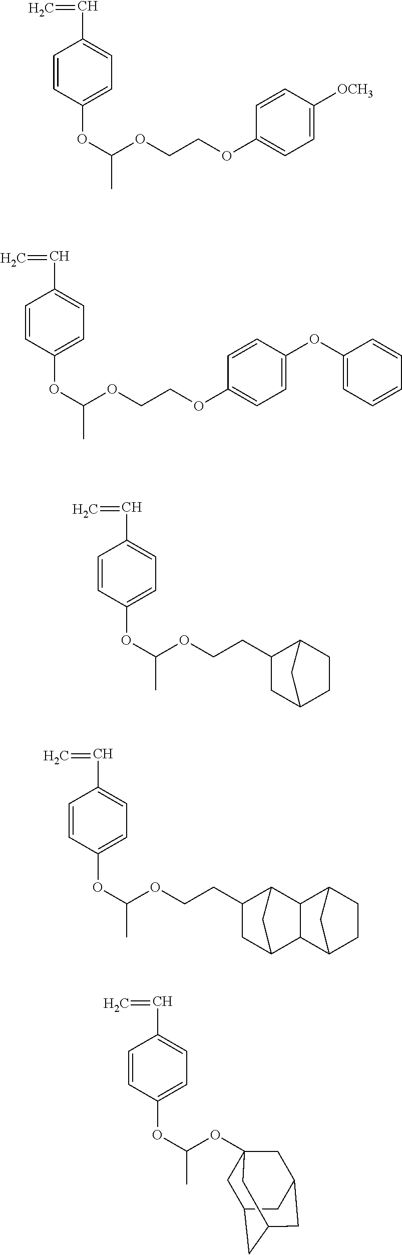

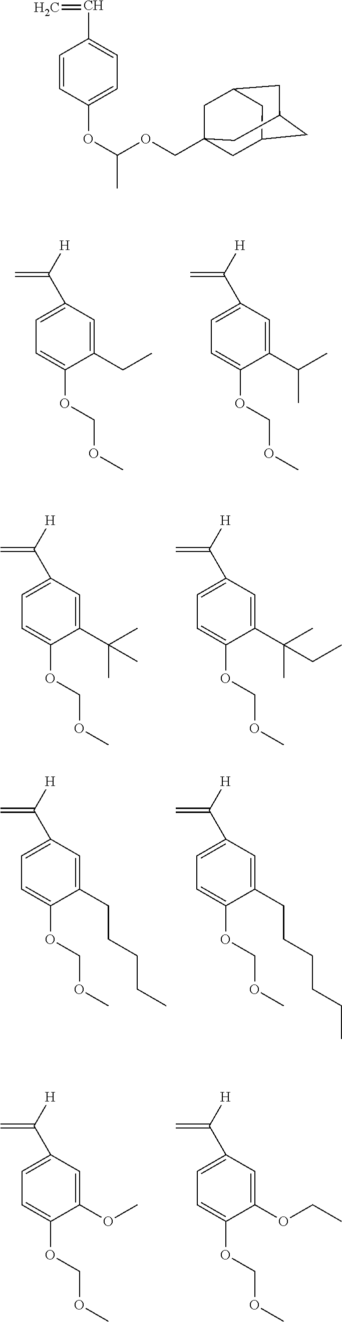

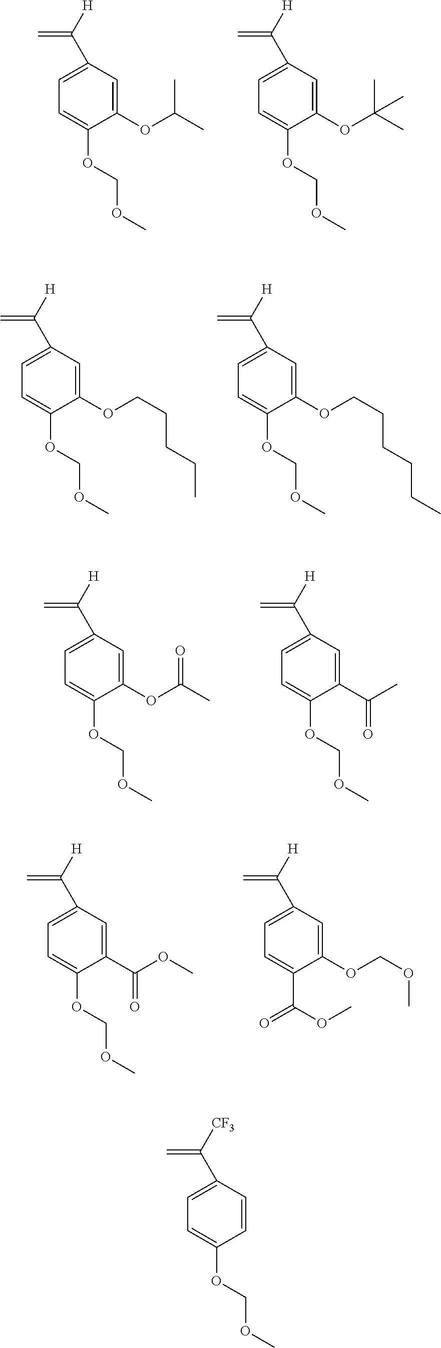

When the resin contains the structural unit derived form the monomer represented by the formula (a1-4), the content of the structural unit derived from the monomer represented by the formula (a1-4) is usually 10 to 95% by mole and preferably 15 to 90% by mole and more preferably 20 to 85% by mole based on total molar of all the structural units of the resin.

The resin can have two or more kinds of structural units derived from the compounds having an acid-labile group.

The resin preferably contains the structural unit derived from the compound having an acid-labile group and a structural unit derived from the compound having no acid-labile group. The resin can have two or more kinds of structural units derived from the compounds having no acid-labile group. When the resin contains the structural unit derived from the compound having an acid-labile group and the structural unit derived from the compound having no acid-labile group, the content of the structural unit derived from the compound having an acid-labile group is usually 10 to 80% by mole and preferably 20 to 60% by mole based on total molar of all the structural units of the resin. The content of the structural unit derived from a monomer having an adamantyl group, especially the monomer represented by the formula (a1-1) in the structural unit derived from the compound having no acid-labile group is preferably 15% by mole or more from the viewpoint of dry-etching resistance of the photoresist composition.

The compound having no acid-labile group preferably contains one or more hydroxyl groups or a lactone ring. When the resin contains the structural unit derived from the compound having no acid-labile group and having one or more hydroxyl groups or a lactone ring, a photoresist composition having good resolution and adhesiveness of photoresist to a substrate tends to be obtained.

Examples of the compound having no acid-labile group and having one or more hydroxyl groups include a monomer represented by the formula (a2-0):

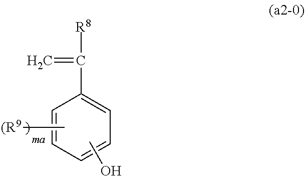

wherein R⁸ represents a hydrogen atom, a halogen atom, a C1-C6 alkyl group or a C1-C6 halogenated alkyl group, R⁹ is independently in each occurrence a halogen atom, a hydroxyl group, a C1-C6 alkyl group, a C1-C6 alkoxy group, a C2-C4 acyl group, a C2-C4 acyloxy group, an acryloyl group or a methacryloyl group, ma represents an integer of 0 to 4, and
a monomer represented by the formula (a2-1):

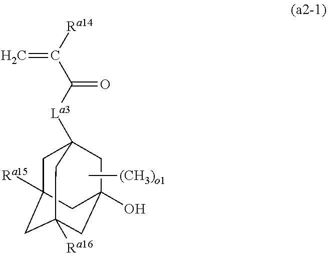

wherein R^{a14} represents a hydrogen atom or a methyl group, R^{a15} and R^{a16} each independently represent a hydrogen atom, a methyl group or a hydroxyl group, L^{a3} represents *—O— or *—O—(CH₂)ₖ₂—CO—O— in which * represents a binding position to —CO—, and k2 represents an integer of 1 to 7, and o1 represents an integer of 0 to 10.

When KrF excimer laser (wavelength: 248 nm) lithography system, or a high energy laser such as electron beam and extreme ultraviolet is used as an exposure system, the resin containing the structural unit derived from the monomer represented by the formula (a2-0) is preferable, and when ArF excimer laser (wavelength: 193 nm) is used as an exposure system, the resin containing the structural unit derived from the monomer represented by the formula (a2-1) is preferable.

In the formula (a2-0), examples of the halogen atom include a fluorine atom, examples of the C1-C6 alkyl group include a methyl group, an ethyl group, a propyl group, an isopropyl group, a butyl group, an isobutyl group, a sec-butyl group, a tert-butyl group, a pentyl group and a hexyl group, and a C1-C4 alkyl group is preferable and a C1-C2 alkyl group is more preferable and a methyl group is especially preferable. Examples of the C1-C6 halogenated alkyl group include a trifluoromethyl group, a pentafluoroethyl group, a heptafluoropropyl group, a heptafluoroisopropyl group, a nonafluorobutyl group, a nonafluoro-sec-butyl group, a nonafluoro-tert-butyl group, a perfluoropentyl group and a perfluorohexyl group. Examples of the C1-C6 alkoxy group include a methoxy group, an ethoxy group, a propoxy group, an isopropoxy group, a butoxy group, an isobutoxy group, a sec-butoxy group, a tert-butoxy group, a pentyloxy group and a hexyloxy group, and a C1-C4 alkoxy group is preferable and a C1-C2 alkoxy group is more preferable and a methoxy group is especially preferable. Examples of the C2-C4 acyl group include an acetyl group, a propionyl group and a butyryl group, and examples of the C2-C4 acyloxy group include an acetyloxy group, a propionyloxy group and a butyryloxy group. In the formula (a2-0), ma is preferably 0, 1 or 2, and is more preferably 0 or 1, and especially preferably 0.

The resin containing the structural unit derived from the monomer represented by the formula (a2-0) and the structural unit derived from the compound having an acid generator can be produced, for example, by polymerizing the compound having an acid generator and a monomer obtained by protecting a hydroxyl group of the monomer represented by the formula (a2-0) with an acetyl group followed by conducting deacetylation of the obtained polymer with a base.

Examples of the monomer represented by the formula (a2-0) include the followings.

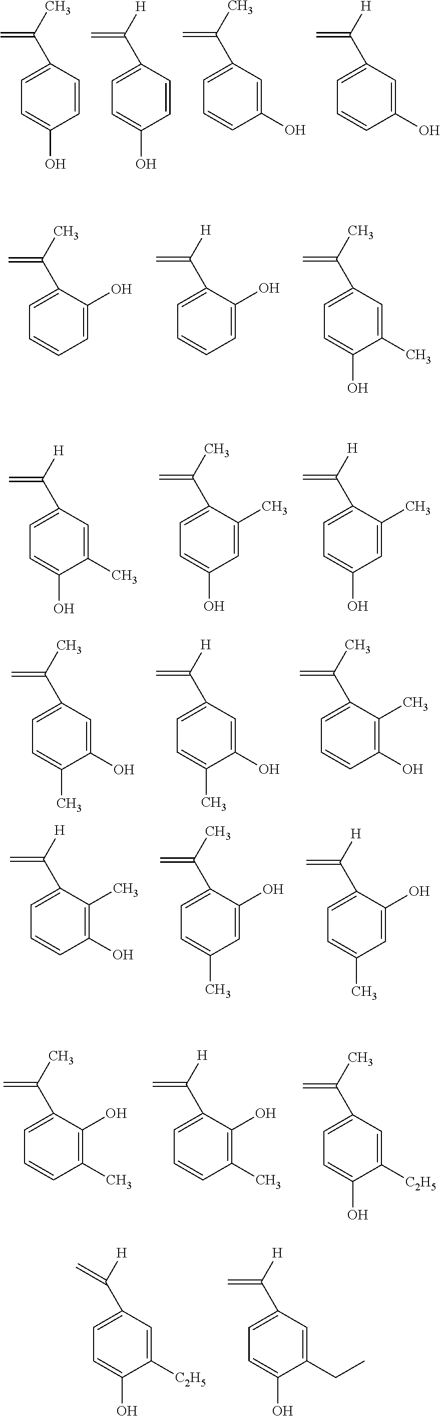

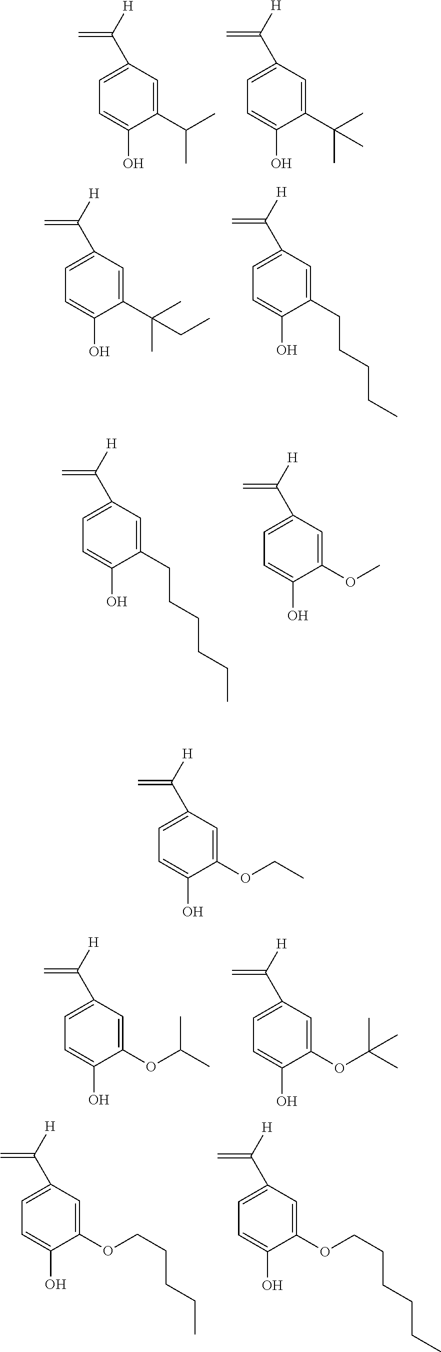

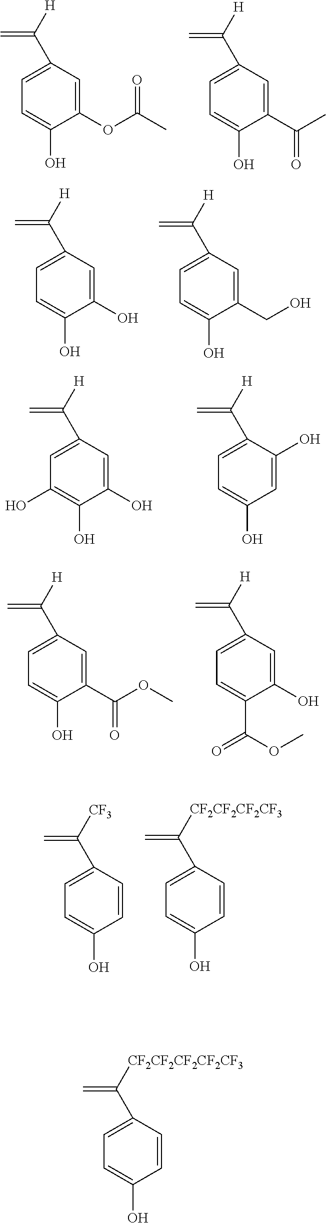

Among them, preferred are 4-hydroxystyrene and 4-hydroxy-α-methylstyrene.

When the resin contains the structural unit derived from the monomer represented by the formula (a2-0), the content of the structural unit derived from the monomer represented by the formula (a2-0) is usually 5 to 90% by mole and preferably 10 to 85% by mole and more preferably 15 to 80% by mole based on total molar of all the structural units of the resin.

In the formula (a2-1), R^{a14} is preferably a methyl group, R^{a15} is preferably a hydrogen atom, R^{a16} is preferably a hydrogen atom or a hydroxyl group, L^{a3} is preferably *—O— or *—O—(CH₂)_{f2}—CO—O— in which * represents a binding position to —CO—, and f2 represents an integer of 1 to 4, and is more preferably *—O—, and o1 is preferably 0, 1, 2 or 3 and is more preferably 0 or 1.

Examples of the monomer represented by the formula (a2-1) include the followings, and 3-hydroxy-1-adamantyl acrylate, 3-hydroxy-1-adamantyl methacrylate, 3,5-dihydroxy-1-adamantyl acrylate, 3,5-dihydroxy-1-adamantyl methacrylate, 1-(3,5-dihydroxy-1-adamantyloxycarbonyl)methyl acrylate and 1-(3,5-dihydroxy-1-adamantyloxycarbonyl)methyl methacrylate are preferable, and 3-hydroxy-1-adamantyl methacrylate and 3,5-dihydroxy-1-adamantyl methacrylate are more preferable.

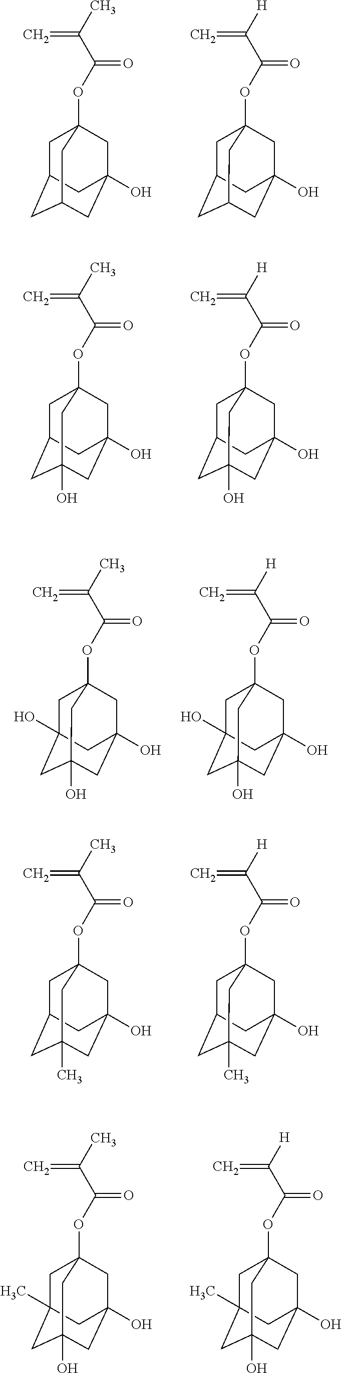

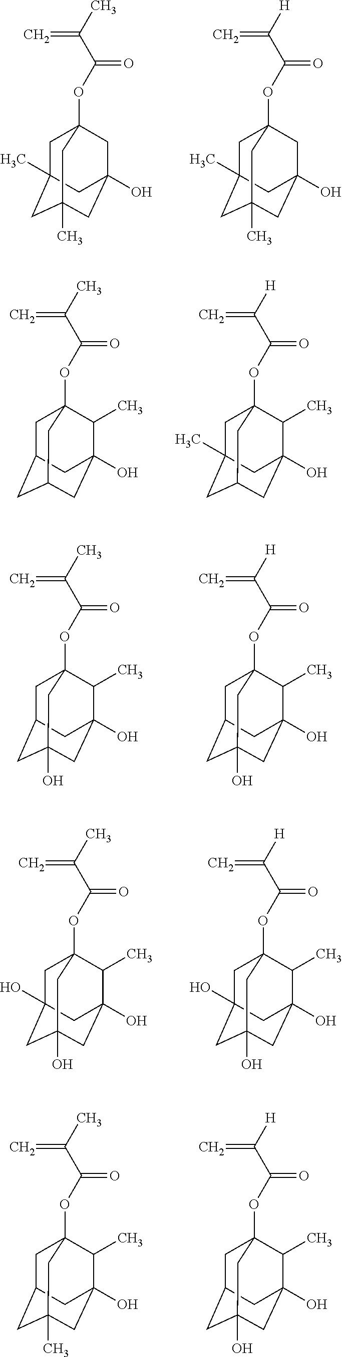

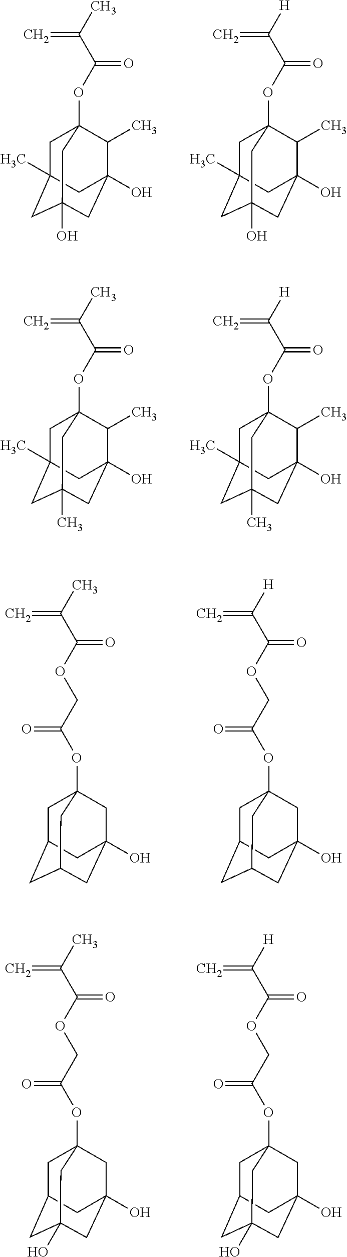

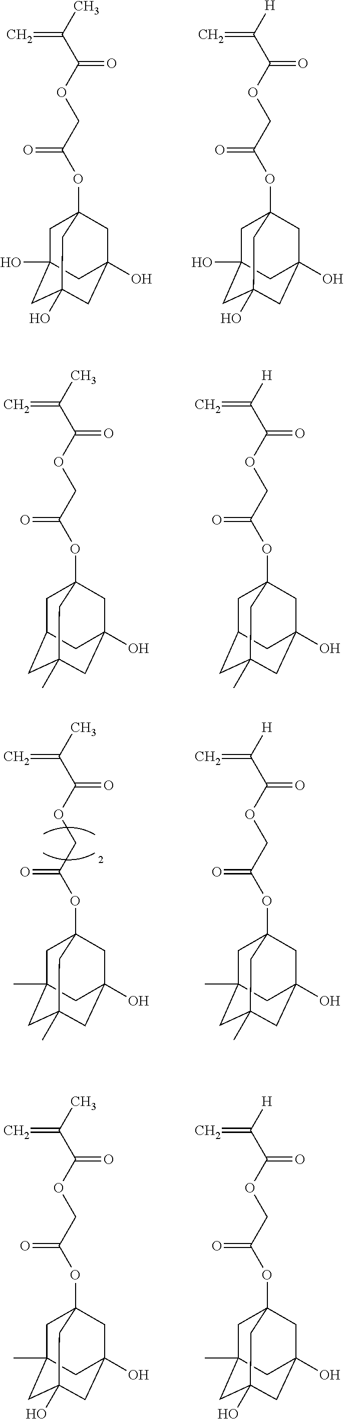

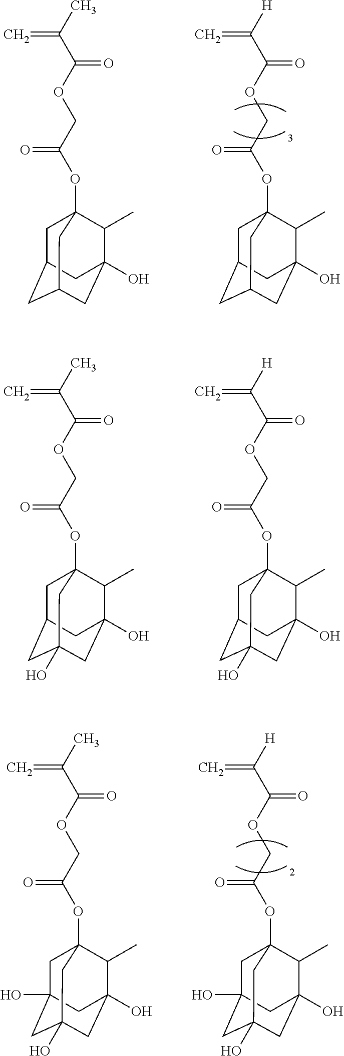

When the resin contains the structural unit derived from the monomer represented by the formula (a2-1), the content of the structural unit derived from the monomer represented by the formula (a2-1) is usually 5 to 50% by mole and preferably 10 to 45% by mole and more preferably 15 to 40% by mole based on total molar of all the structural units of the resin.

Examples of the lactone ring of the compound having no acid-labile group and having a lactone ring include a monocyclic lactone ring such as β-propiolactone ring, γ-butyrolactone ring and γ-valerolactone ring, and a condensed ring formed from a monocyclic lactone ring and the other ring. Among them, preferred are γ-butyrolactone ring and a condensed lactone ring formed from γ-butyrolactone ring and the other ring.

Preferable examples of the monomer having no acid-labile group and a lactone ring include the monomers represented by the formulae (a3-1), (a3-2) and (a3-3):

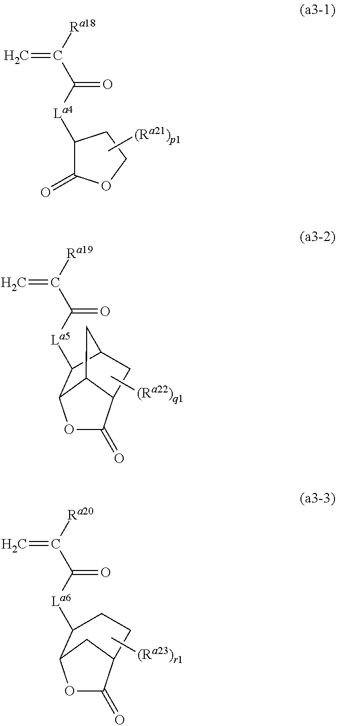

wherein L^{a4}, L^{a5} and L^{a6} each independently represent *—O— or *—O—(CH₂)ₖ₃—CO—O— in which * represents a binding position to —CO— and k3 represents an integer of 1 to 7, R^{a18}, R^{a19} and R^{a20} each independently represent a hydrogen atom or a methyl group, R^{a21} represents a C1-C4 aliphatic hydrocarbon group, R^{a22} and R^{a23} are independently in each occurrence a carboxyl group, a cyano group or a C1-C4 aliphatic hydrocarbon group, and p1 represents an integer of 0 to 5, q1 and r1 independently each represent an integer of 0 to 3.

It is preferred that L^{a4}, L^{a5} and L^{a6} each independently represent *—O— or *—O—(CH₂)_{d1}—CO—O— in which * represents a binding position to —CO— and d1 represents an integer of 1 to 4, and it is more preferred that L^{a4}, L^{a5} and L^{a6} are *—O—. R^{a18}, R^{a19} and R^{a20} are preferably methyl groups. R^{a21} is preferably a methyl group. It is preferred that R^{a22} and R^{a23} are independently in each occurrence a carboxyl group, a cyano group or a methyl group. It is preferred that p1 is an integer of 0 to 2, and it is more preferred that p1 is 0 or 1. It is preferred that q1 and r1 independently each represent an integer of 0 to 2, and it is more preferred that q1 and r1 independently each represent 0 or 1.

Examples of the monomer represented by the formula (a3-1) include the followings.

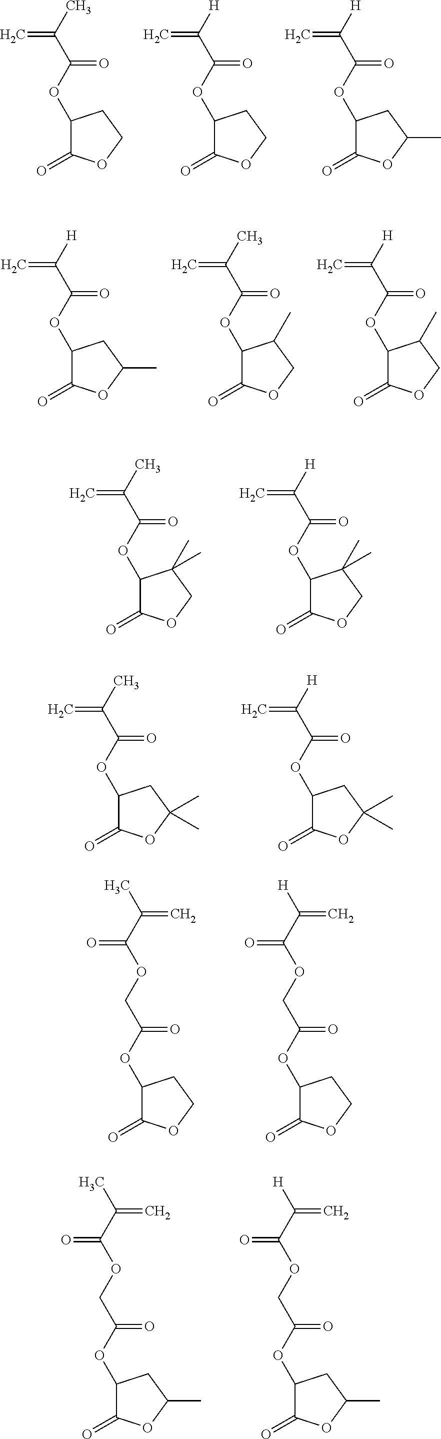

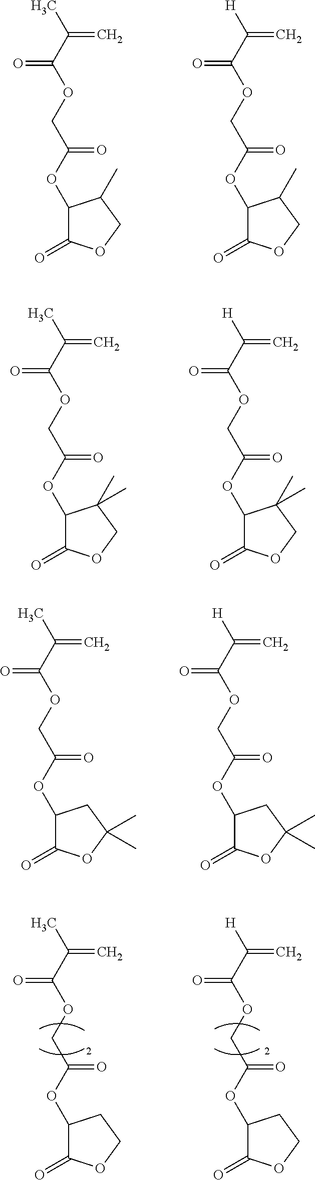

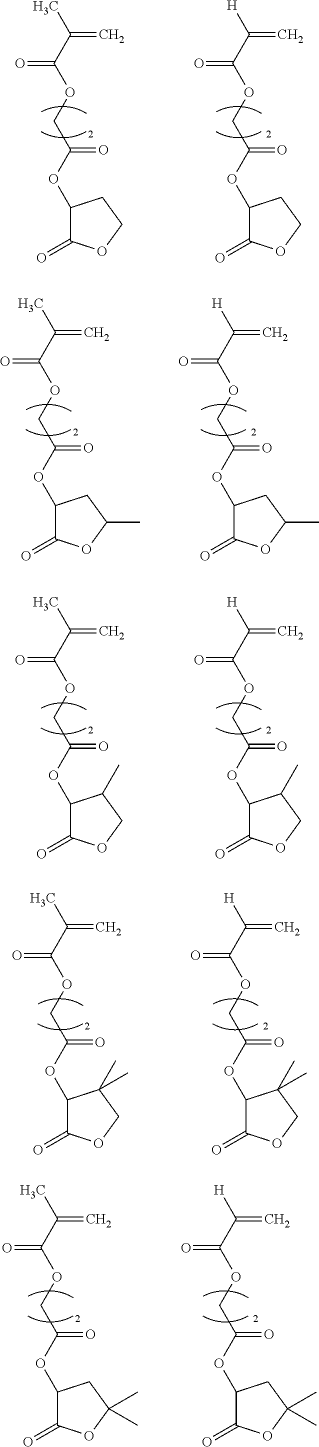

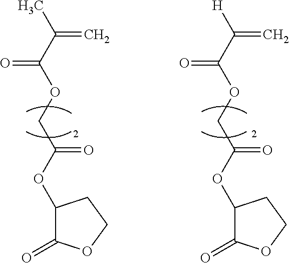

Examples of the monomer represented by the formula (a3-2) include the followings.

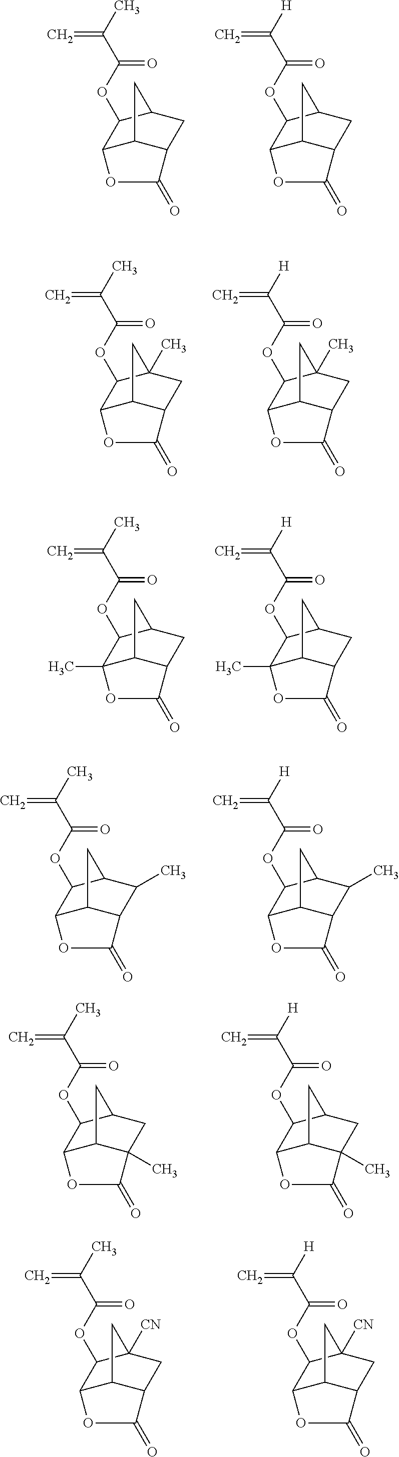

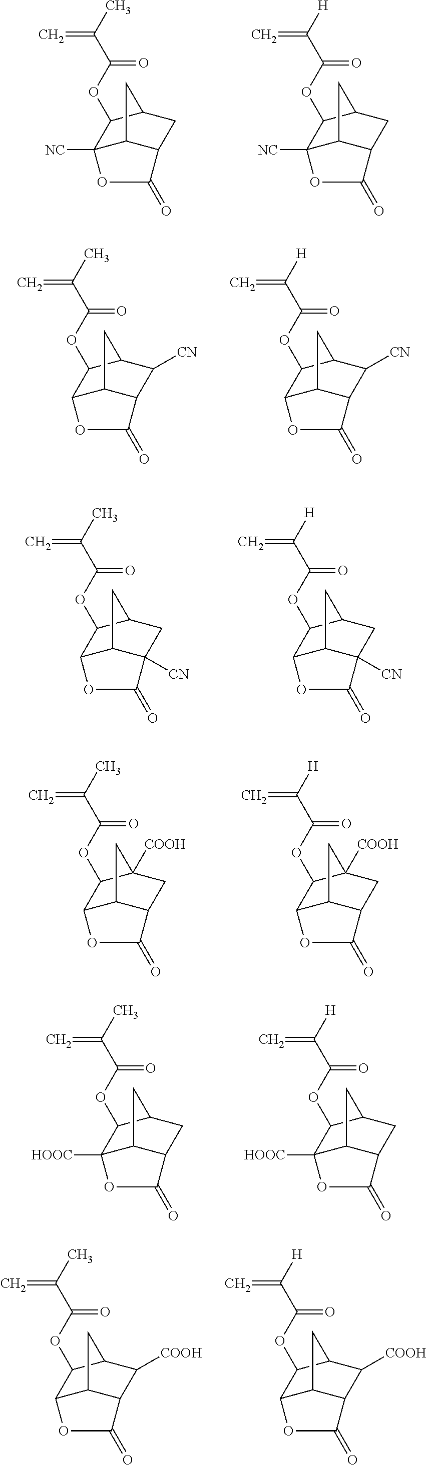

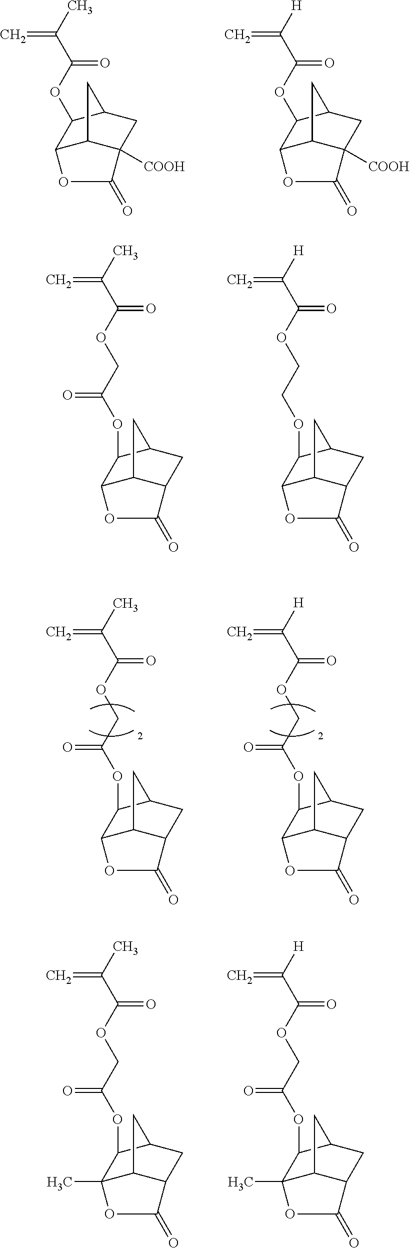

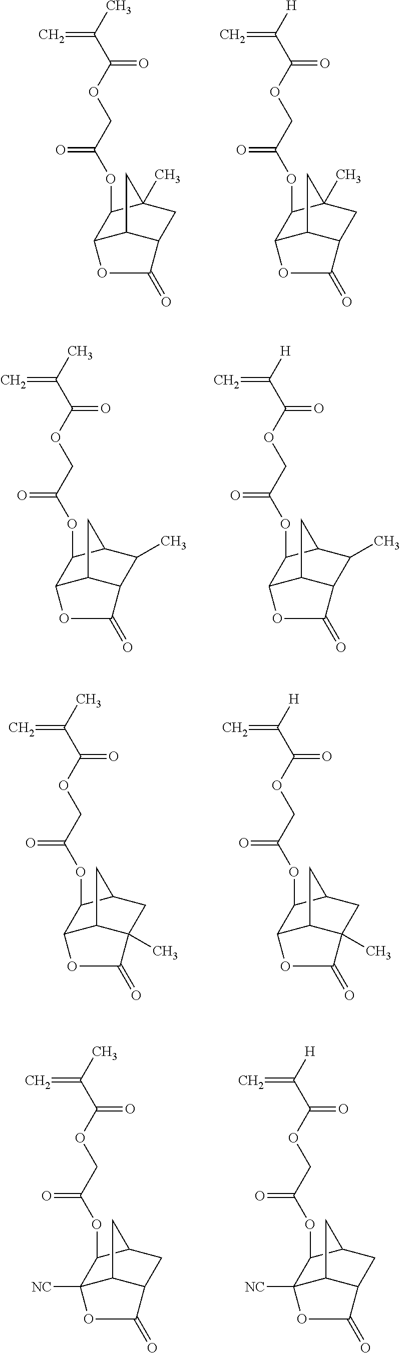

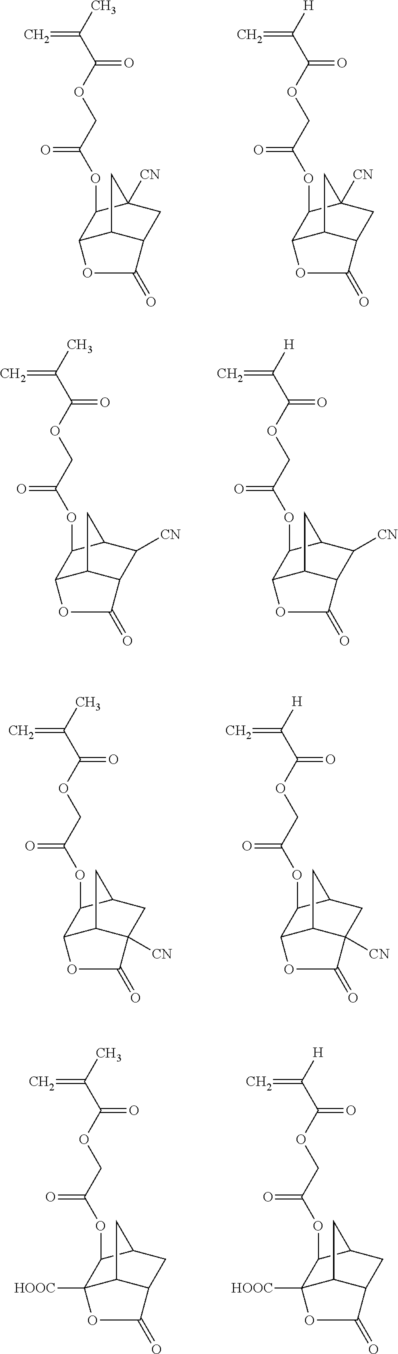

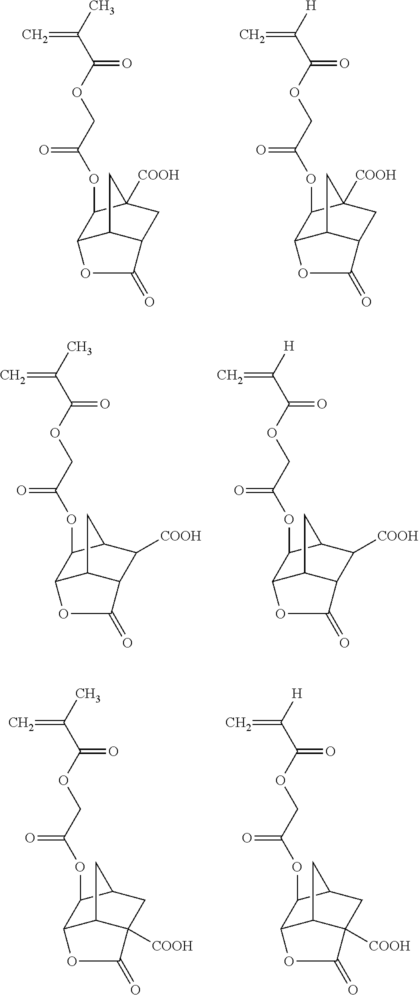

Examples of the monomer represented by the formula (a3-3) include the followings.

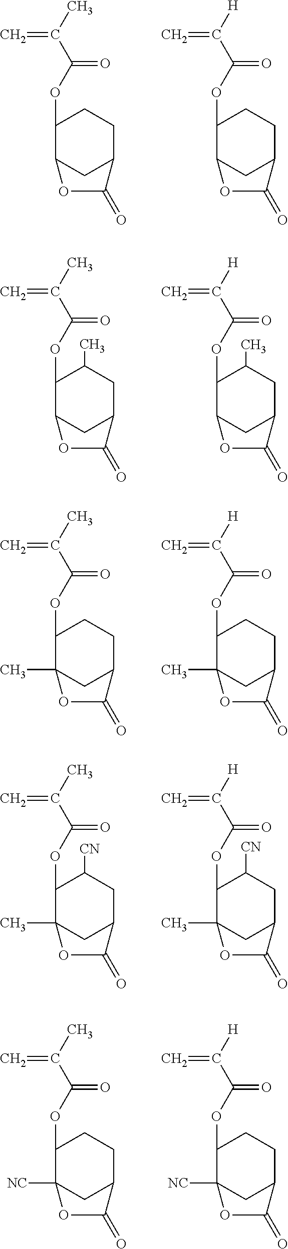

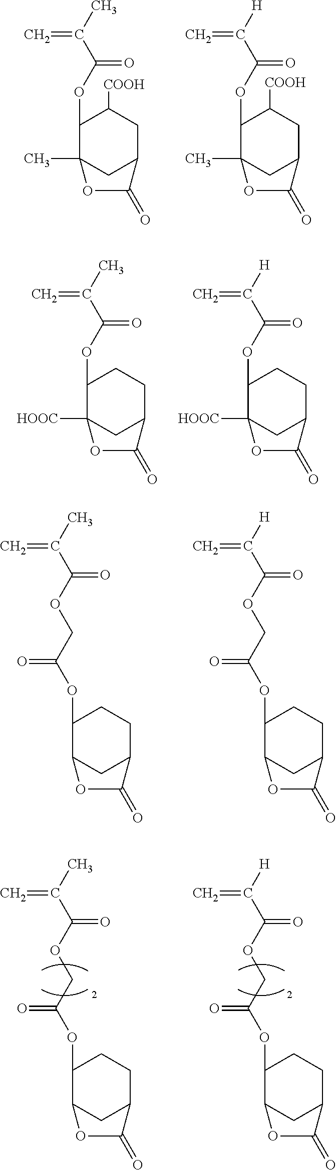

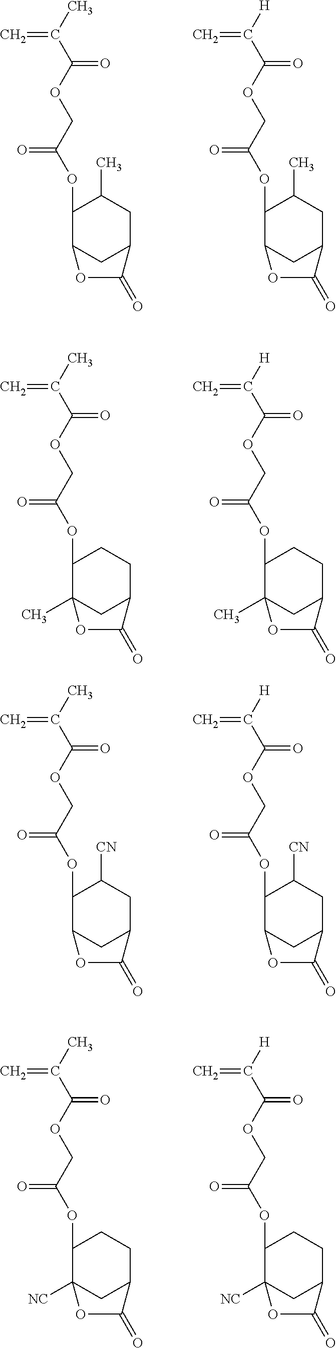

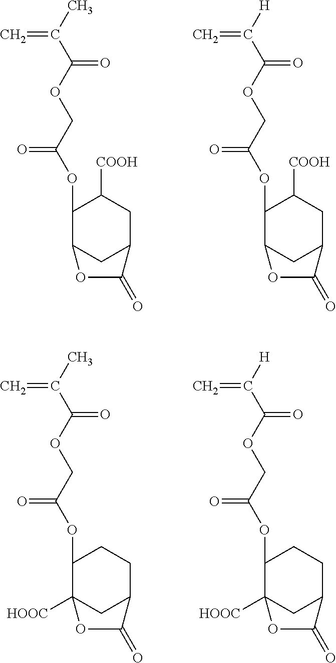

Among them, preferred are 5-oxo-4-oxatricyclo[4.2.1.0^{3,7}]nonan-2-yl acrylate, 5-oxo-4-oxatricyclo[4.2.1.0^{3,7}]nonan-2-yl methacrylate, tetrahydro-2-oxo-3-furyl acrylate, tetrahydro-2-oxo-3-furyl methacrylate, 2-(5-oxo-4-oxatricyclo[4.2.1.0^{3,7}]nonan-2-yloxy)-2-oxoethyl acrylate and 2-(5-oxo-4-oxatricyclo[4.2.1.0^{3,7}]nonan-2-yloxy)-2-oxoethyl methacrylate, and more preferred are 5-oxo-4-oxatricyclo[4.2.1.0^{3,7}]nonan-2-yl methacrylate, tetrahydro-2-oxo-3-furyl methacrylate and 2-(5-oxo-4-oxatricyclo[4.2.1.0^{3,7}]nonan-2-yloxy)-2-oxoethyl methacrylate.

When the resin contains the structural unit derived from the monomer having no acid-labile group and having a lactone ring, the content thereof is usually 5 to 50% by mole and preferably 10 to 45% by mole and more preferably 15 to 40% by mole based on total molar of all the structural units of the resin.

The resin can contain a structural unit derived from a monomer having an acid labile group containing a lactone ring. Examples of the monomer having an acid labile group containing a lactone ring include the followings.

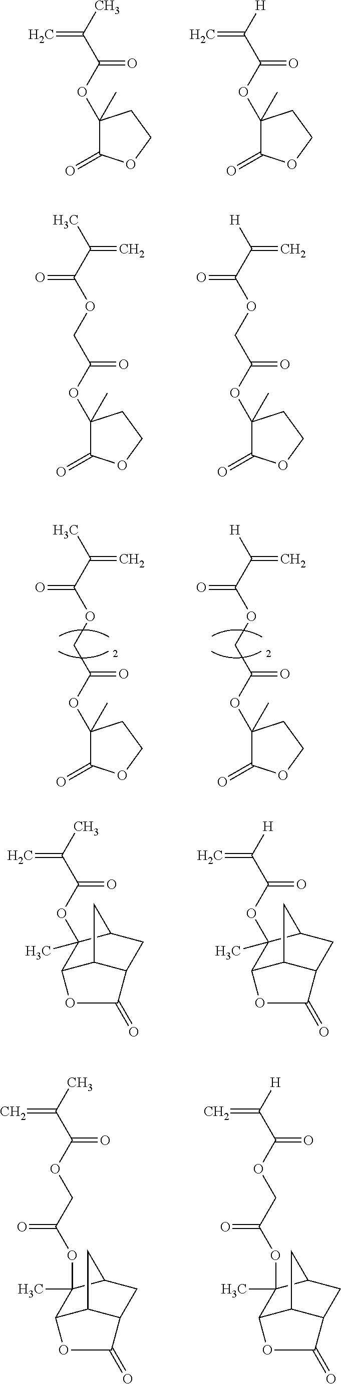

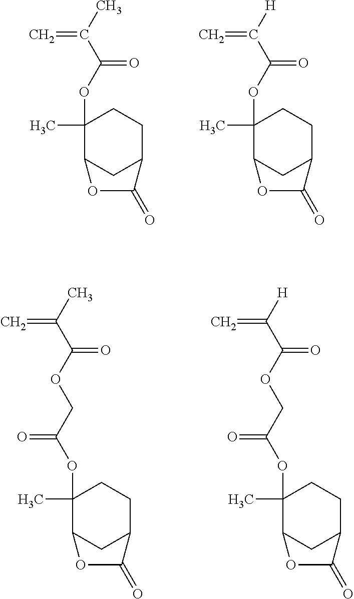

Examples of the other monomer having no acid-labile group include the monomers represented by the formulae (a4-1), (a4-2) and (a4-3):

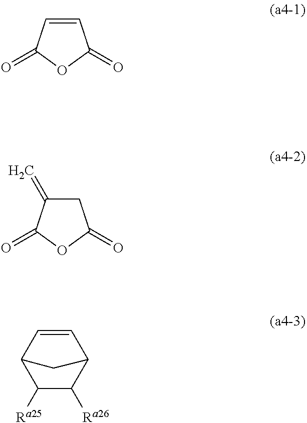

wherein R^{a25} and R^{a26} each independently represents a hydrogen atom, a C1-C3 aliphatic hydrocarbon group which can have one or more substituents, a carboxyl group, a cyano group or a —COOR^{a27} group in which R^{a27} represents a C1-C36 aliphatic hydrocarbon group or a C3-C36 saturated cyclic hydrocarbon group, and one or more —CH₂— in the C1-C36 aliphatic hydrocarbon group and the C3-C36 saturated cyclic hydrocarbon group can be replaced by —O— or —CO—, with the proviso that the carbon atom bonded to —O— of —COO— of R^{a27} is not a tertiary carbon atom, or R^{a25} and R^{a26} are bonded together to form a carboxylic anhydride residue represented by —C(═O)OC(═O)—.

Examples of the substituent of the C1-C3 aliphatic hydrocarbon group include a hydroxyl group. Examples of the C1-C3 aliphatic hydrocarbon group which can have one or more substituents include a C1-C3 alkyl group such as a methyl group, an ethyl group and a propyl group, and a C1-C3 hydroxyalkyl group such a hydroxymethyl group and a 2-hydroxyethyl group. The C1-C36 aliphatic hydrocarbon group represented by R^{a27} is preferably a C1-C8 aliphatic hydrocarbon group and is more preferably a C1-C6 aliphatic hydrocarbon group. The C3-C36 saturated cyclic hydrocarbon group represented by R^{a27} is preferably a C4-C36 saturated cyclic hydrocarbon group, and is more preferably C4-C12 saturated cyclic hydrocarbon group. Examples of R^{a27} include a methyl group, an ethyl group, a propyl group, a 2-oxo-oxolan-3-yl group and a 2-oxo-oxolan-4-yl group.

Examples of the monomer represented by the formula (a4-3) include 2-norbornene, 2-hydroxy-5-norbornene, 5-norbornene-2-carboxylic acid, methyl 5-norbornene-2-carboxylate, 2-hydroxyethyl 5-norbornene-2-carboxylate, 5-norbornene-2-methanol and 5-norbornene-2,3-dicarboxylic anhydride.

When the resin contains a structural unit derived from a monomer represented by the formula (a4-1), (a4-2) or (a4-3), the content thereof is usually 2 to 40% by mole and preferably 3 to 30% by mole and more preferably 5 to 20% by mole based on total molar of all the structural units of the resin.

Preferable resin is a resin containing the structural units derived from the monomer having an acid-labile group, and the structural units derived from the monomer having one or more hydroxyl groups and/or the monomer having a lactone ring. The monomer having an acid-labile group is preferably the monomer represented by the formula (a1-1) or the monomer represented by the formula (a1-2), and is more preferably the monomer represented by the formula (a1-1). The monomer having one or more hydroxyl groups is preferably the monomer represented by the formula (a2-1), and the monomer having a lactone ring is preferably the monomer represented by the formula (a3-1) or (a3-2).

The resin can be produced according to known polymerization methods such as radical polymerization.

The resin usually has 2,500 or more of the weight-average molecular weight, and preferably 3,000 or more of the weight-average molecular weight. The resin usually has 50,000 or less of the weight-average molecular weight, and preferably has 30,000 or less of the weight-average molecular weight. The weight-average molecular weight can be measured with gel permeation chromatography.

The content of the resin is usually 80% by weight or more in the solid component.

The photoresist composition of the present invention contains an acid generator, and preferably a photoacid generator.

The acid generator is a substance which is decomposed to generate an acid by applying a radiation such as a light, an electron beam or the like on the substance itself or on a photoresist composition containing the substance. The acid generated from the acid generator acts on the resin resulting in cleavage of the acid-labile group existing in the resin.

Examples of the acid generator include a nonionic acid generator, an ionic acid generator and the combination thereof. An ionic acid generator is preferable. Examples of the nonionic acid generator include an organo-halogen compound, a sulfone compound such as a disulfone, a ketosulfone and a sulfonyldiazomethane, a sulfonate compound such as a 2-nitrobenzylsulfonate, an aromatic sulfonate, an oxime sulfonate, an N-sulfonyloxyimide, a sulfonyloxyketone and DNQ 4-sulfonate. Examples of the ionic acid generator include an acid generator having an inorganic anion such as BF₄⁻, PF₆⁻, AsF₆⁻ and SbF₆⁻, and an acid generator having an organic anion such as a sulfonic acid anion and a bissulfonylimido anion, and an acid generator having a sulfonic acid anion is preferable. Preferable examples of the acid generator include a salt represented by the formula (B1):

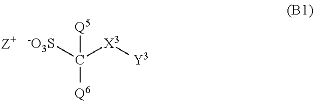

wherein Q⁵ and Q⁶ each independently represent a fluorine atom or a C1-C6 perfluoroalkyl group, X³ represents a single bond or a C1-C17 divalent saturated hydrocarbon group in which one or more —CH₂— can be replaced by —O— or —CO—,
Y³ represents a C1-C36 aliphatic hydrocarbon group which can have one or more substituents, a C3-C36 saturated cyclic hydrocarbon group which can have one or more substituents, or a C6-C36 aromatic hydrocarbon group which can have one or more substituents, and one or more —CH₂— in the aliphatic hydrocarbon group, the saturated cyclic hydrocarbon group and the aromatic hydrocarbon group can be replaced by —O—, —CO— or —SO₂—, and Z⁺ represents an organic cation.

Examples of the C1-C6 perfluoroalkyl group include the same as described in Q¹ and Q², and a trifluoromethyl group is preferable. Q⁵ and Q⁶ each independently preferably represent a fluorine atom or a trifluoromethyl group, and Q⁵ and Q⁶ are more preferably fluorine atoms.

Examples of X³ include the same as X¹, and examples of Y³ include the same as Y¹.

Examples of the anion part of the salt represented by the formula (B1) include the anions derived from the above-mentioned acids represented by the formulae (IA-1) to (IA-310). The anions derived from the above-mentioned acids represented by the formulae (IA-1) to (IA-310) are anion wherein —SO₃H in the above-mentioned acids represented by the formulae (IA-1) to (IA-310) are converted to —SO₃⁻.

Examples of the cation part represented by Z⁺ include an onium cation such as a sulfonium cation, an iodonium cation, an ammonium cation, a benzothiazolium cation and a phosphonium cation, and a sulfonium cation and an iodonium cation are preferable, and an arylsulfonium cation is more preferable.

Preferable examples of the cation part represented by Z⁺ include the cations represented by the formulae (b2-1) to (b2-4):

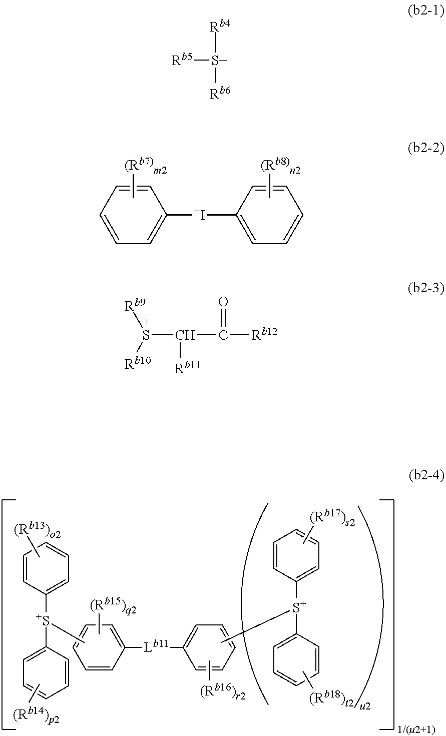

wherein R^{b4}, R^{b5} and R^{b6} each independently represent a C1-C30 aliphatic hydrocarbon group which can have one or more substituents selected from the group consisting of a hydroxyl group, a C1-C12 alkoxy group and a C6-C18 aromatic hydrocarbon group, a C3-C36 saturated cyclic hydrocarbon group which can have one or more substituents selected from the group consisting of a halogen atom, a C2-C4 acyl group and a glycidyloxy group, or a C6-C18 aromatic hydrocarbon group which can have one or more substituents selected from the group consisting of a halogen atom, a hydroxyl group, a C1-C18 aliphatic hydrocarbon group, a C3-C36 saturated cyclic hydrocarbon group or a C1-C12 alkoxy group,
R^{b7} and R^{b8} are independently in each occurrence a hydroxyl group, a C1-C12 aliphatic hydrocarbon group or a C1-C12 alkoxy group, m2 and n2 independently represents an integer of 0 to 5,
R^{b9} and R^{b10} each independently represent a C1-C36 aliphatic hydrocarbon group or a C3-C36 saturated cyclic hydrocarbon group, or R^{b9} and R^{b10} are bonded to form a C2-C11 divalent acyclic hydrocarbon group which forms a ring together with the adjacent S⁺, and one or more —CH₂— in the divalent acyclic hydrocarbon group may be replaced by —CO—, —O— or —S—,
and
R^{b11} represents a hydrogen atom, a C1-C36 aliphatic hydrocarbon group, a C3-C36 saturated cyclic hydrocarbon group or a C6-C18 aromatic hydrocarbon group, R^{b12} represents a C1-C12 aliphatic hydrocarbon group, a C3-C18 saturated cyclic hydrocarbon group or a C6-C18 aromatic hydrocarbon group and the aromatic hydrocarbon group can have one or more substituents selected from the group consisting of a C1-C12 aliphatic hydrocarbon group, a C1-C12 alkoxy group, a C3-C18 saturated cyclic hydrocarbon group and an C2-C13 acyloxy group, or R^{b11} and R^{b12} are bonded each other to form a C1-C10 divalent acyclic hydrocarbon group which forms a 2-oxocycloalkyl group together with the adjacent —CHCO—, and one or more —CH₂— in the divalent acyclic hydrocarbon group may be replaced by —CO—, —O— or —S—, and
R^{b13}, R^{b14}, R^{b15}, R^{b16}, R^{b17} and R^{b18} each independently represent a hydroxyl group, a C1-C12 aliphatic hydrocarbon group or a C1-C12 alkoxy group, L^{b1} represents —S— or —O— and o2, p2, s2 and t2 each independently represents an integer of 0 to 5, q2 and r2 each independently represents an integer of 0 to 4, and u2 represents 0 or 1.

The aliphatic hydrocarbon group represented by R^{b9} to R^{b11} has preferably 1 to 12 carbon atoms. The saturated cyclic hydrocarbon group represented by R^{b9} to R^{b11} has preferably 3 to 18 carbon atoms and more preferably 4 to 12 carbon atoms.

Examples of the aliphatic hydrocarbon group and the aromatic hydrocarbon group include the same as described above. Preferable examples of the aliphatic hydrocarbon group include a methyl group, an ethyl group, a propyl group, an isopropyl group, a butyl group, a sec-butyl group, a tert-butyl group, a pentyl group, a hexyl group, an octyl group and a 2-ethylhexyl group. Preferable examples of the saturated cyclic hydrocarbon group include a cyclopropyl group, a cyclobutyl group, a cyclopentyl group, a cyclohexyl group, a cycloheptyl group, a cyclodecyl group, a 2-alkyl-a-adamantyl group, a 1-(1-adamantyl)-1-alkyl group and an isobornyl group. Preferable examples of the aromatic group include a phenyl group, a 4-methylphenyl group, a 4-ethylphenyl group, a 4-tert-butylphenyl group, a 4-cyclohexylphenyl group, a 4-methoxyphenyl group, a biphenyl group and a naphthyl group. Examples of the aliphatic hydrocarbon group having an aromatic hydrocarbon group include a benzyl group. Examples of the alkoxy group include a methoxy group, an ethoxy group, a propoxy group, an isopropoxy group, a butoxy group, a sec-butoxy group, a tert-butoxy group, a pentyloxy group, a hexyloxy group, a heptyloxy group, an octyloxy group, a 2-ethylhexyloxy group, a nonyloxy group, a decyloxy group, an undecyloxy group and a dodecyloxy group.

Examples of the C3-C12 divalent acyclic hydrocarbon group formed by bonding R^{b9} and R^{b10} include a trimethylene group, a tetramethylene group and a pentamethylene group. Examples of the ring group formed together with the adjacent S⁺ and the divalent acyclic hydrocarbon group include a thiolan-1-ium ring (tetrahydrothiphenium ring), a thian-1-ium ring and a 1,4-oxathian-4-ium ring. A C3-C7 divalent acyclic hydrocarbon group is preferable.

Examples of the C1-C10 divalent acyclic hydrocarbon group formed by bonding R^{b11} and R^{b12} include a methylene group, an ethylene group, a trimethylene group, a tetramethylene group and a pentamethylene group and examples of the ring group include the followings.

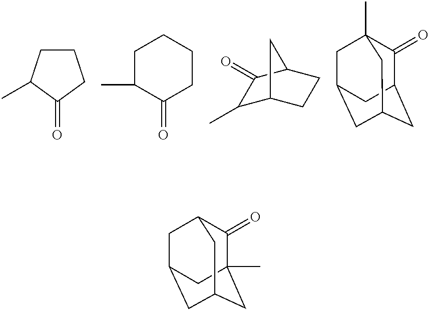

Among the above-mentioned cations, preferred is the cation represented by the formula (b2-1), and more preferred is the cation represented by the formula (b2-1-1), and especially preferred is a triphenylsulfonium cation.

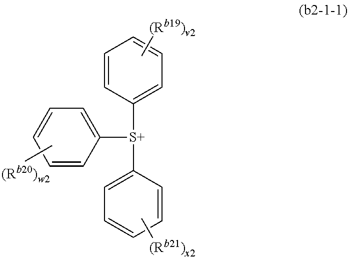

wherein R^{b19}, R^{b20} and R^{b21} are independently in each occurrence a hydroxyl group, a C1-C36 aliphatic hydrocarbon group, a C3-C36 saturated cyclic hydrocarbon group or a C1-C12 alkoxy group, and one or more hydrogen atoms in the aliphatic hydrocarbon group can be replaced by a hydroxyl group, a C1-C12 alkoxy group or a C6-C18 aromatic hydrocarbon group, one or more hydrogen atoms of the saturated cyclic hydrocarbon group can be replaced by a halogen atom, a C2-C4 acyl group or a glycidyloxy group, and v2, w2 and x2 independently each represent an integer of 0 to 5. The aliphatic hydrocarbon group preferably has 1 to 12 carbon atoms, and the saturated cyclic hydrocarbon group preferably has 4 to 36 carbon atoms, and it is preferred that v2, w2 and x2 independently each represent 0 or 1. It is preferred that R^{b19}, R^{b20} and R^{b21} are independently halogen atom (preferably a chlorine atom), a hydroxyl group, a C1-C12 alkyl group or a C1-C12 alkoxy group.

Examples of the cation represented by the formula (b2-1) include the followings.

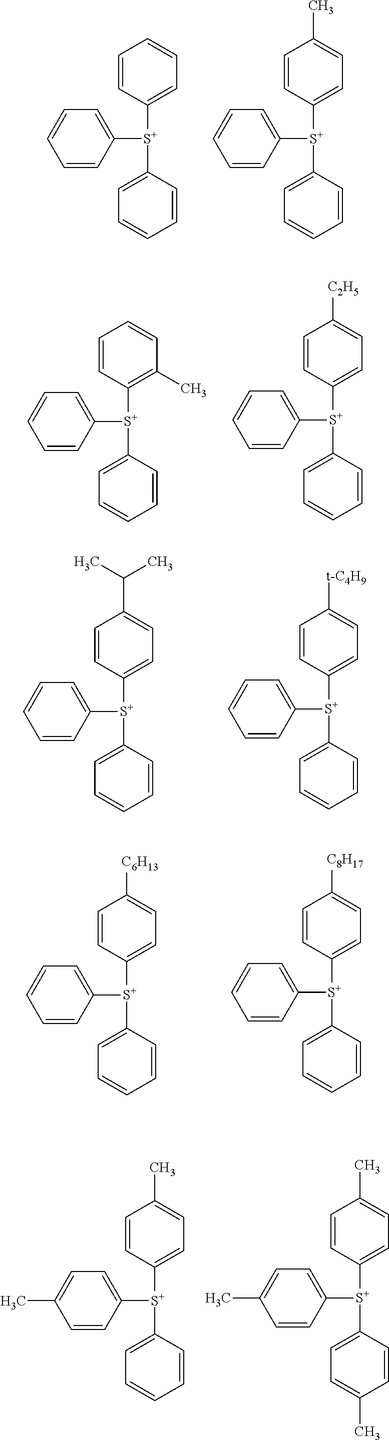

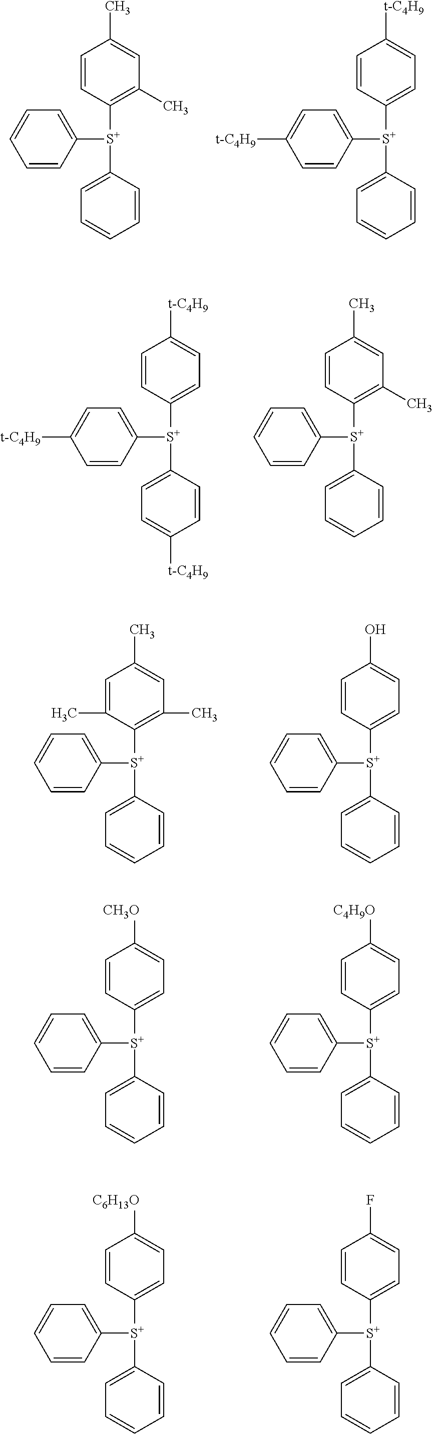

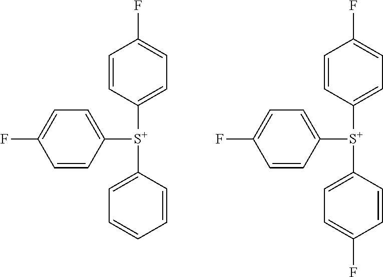

Examples of the cation represented by the formula (b2-2) include the followings.

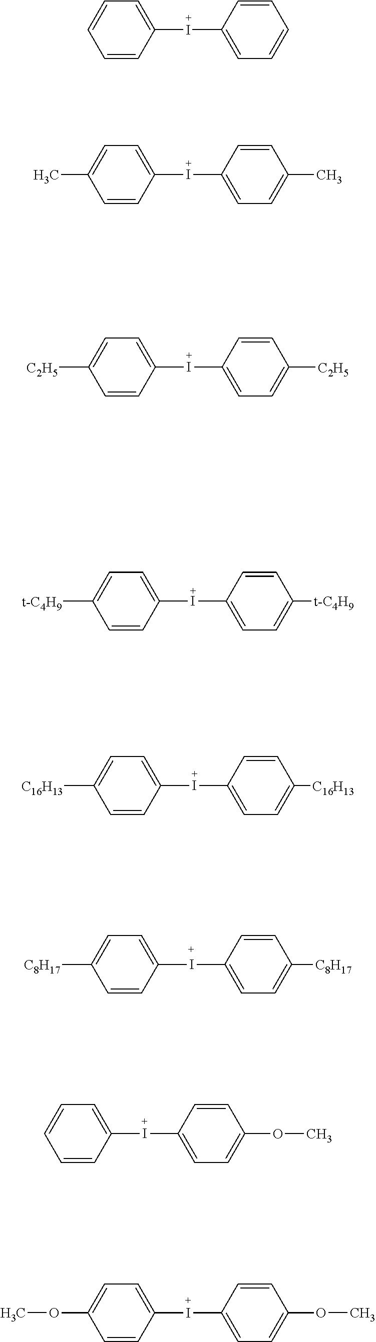

Examples of the cation represented by the formula (b2-3) include the followings.

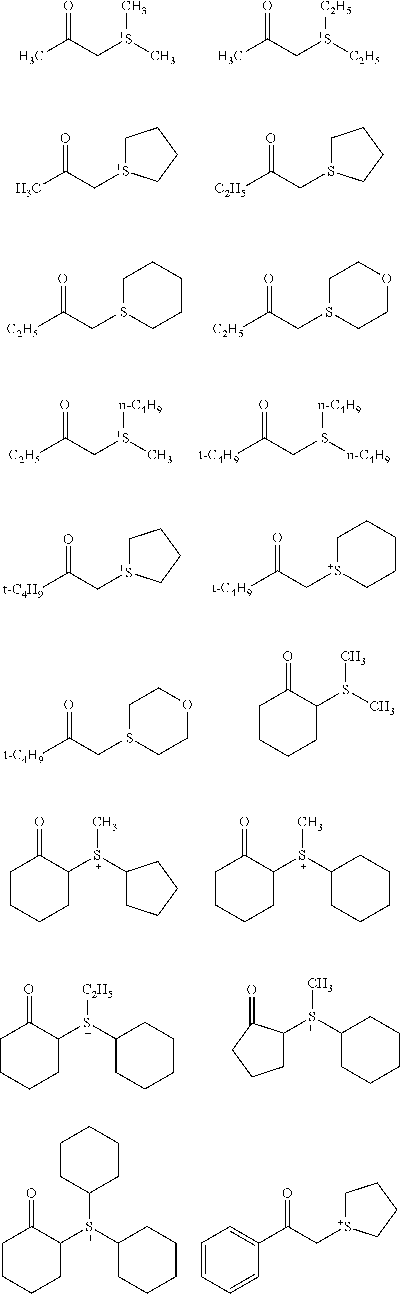

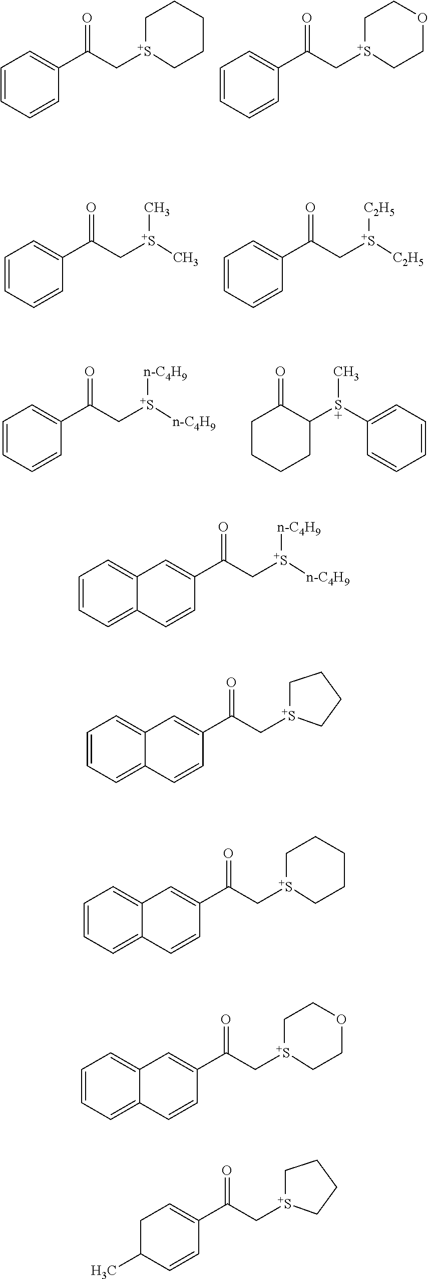

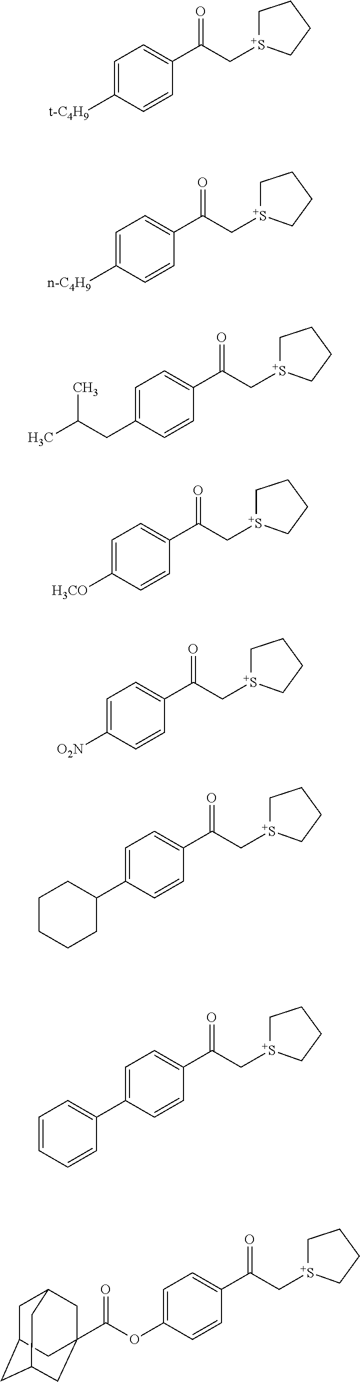

Examples of the cation represented by the formula (b2-4) include the followings.

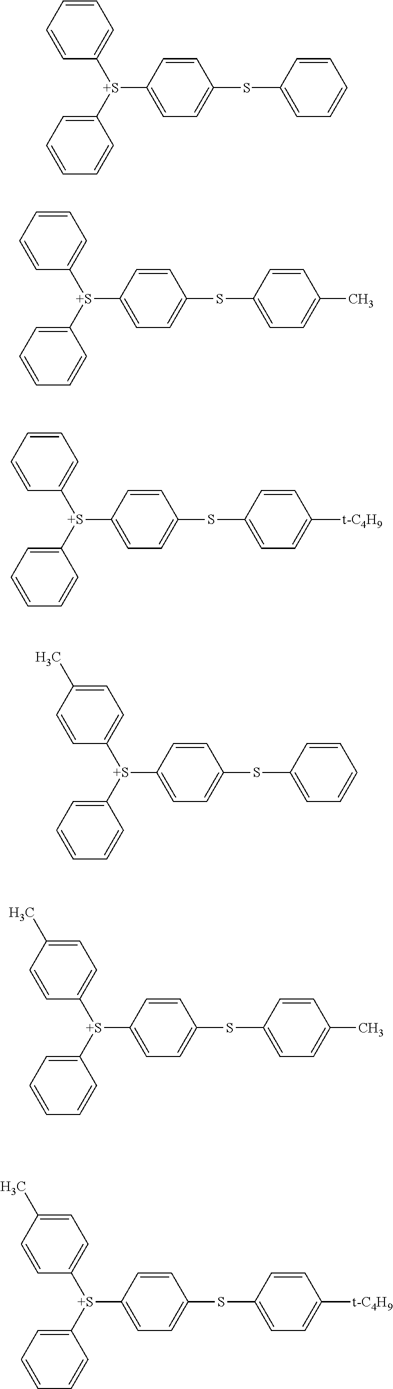

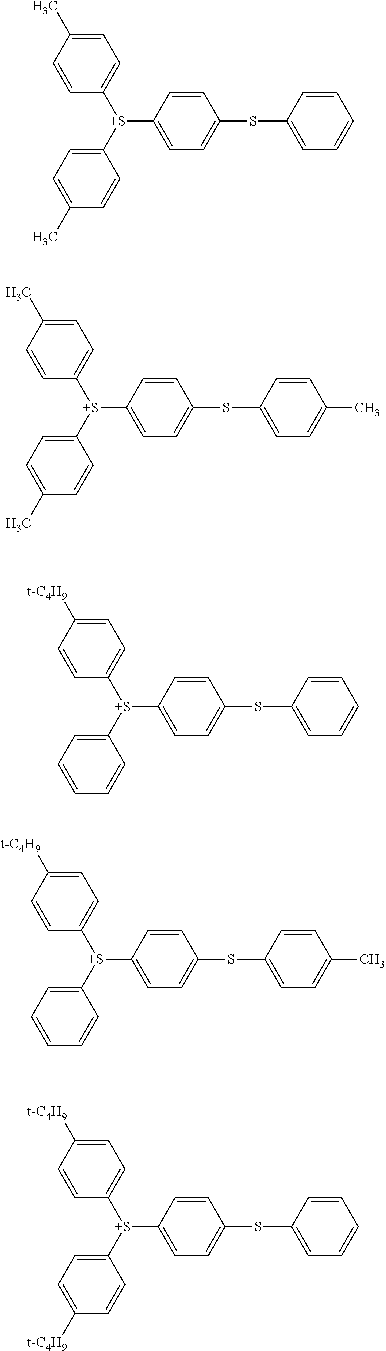

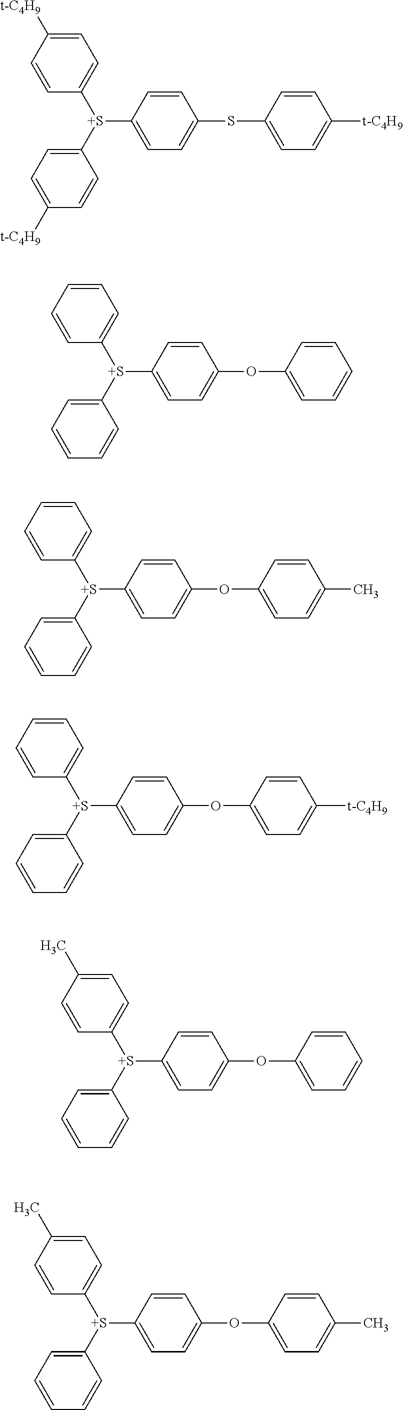

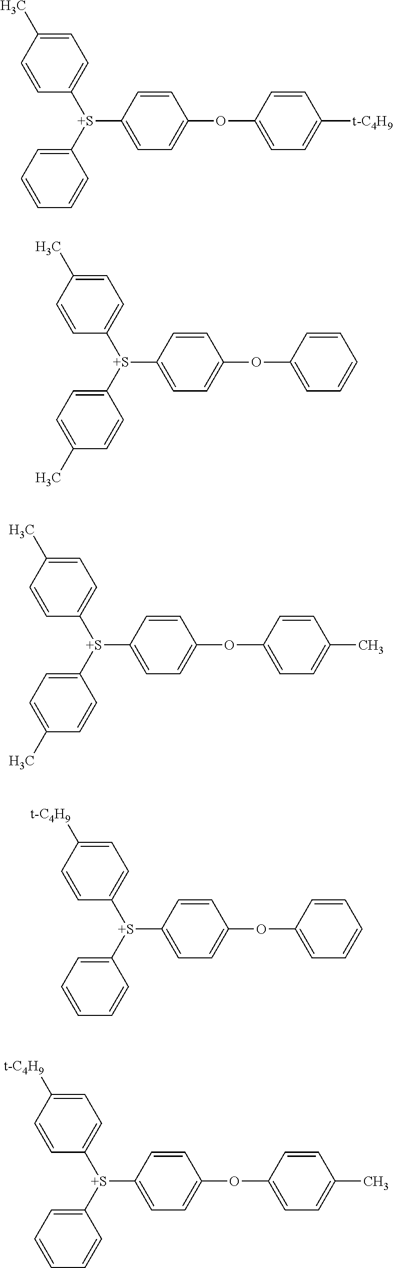

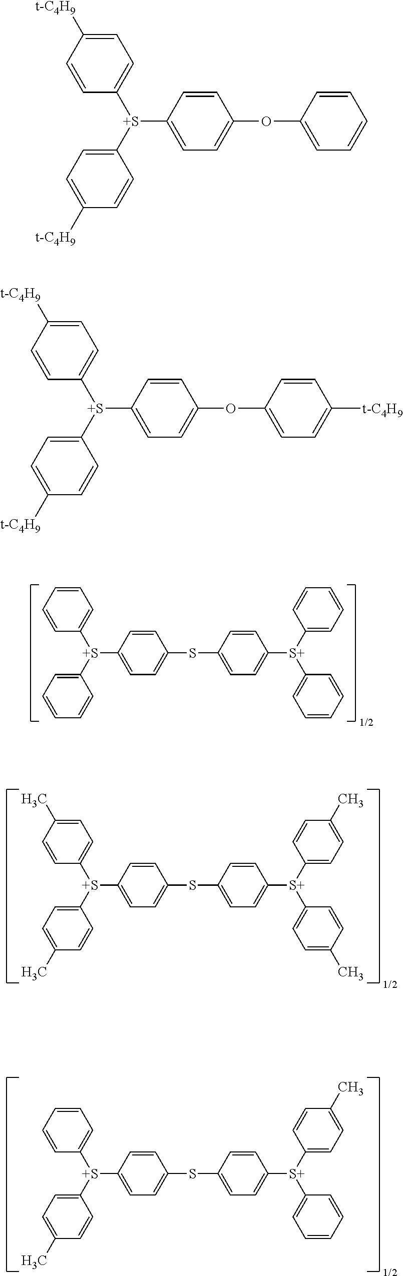

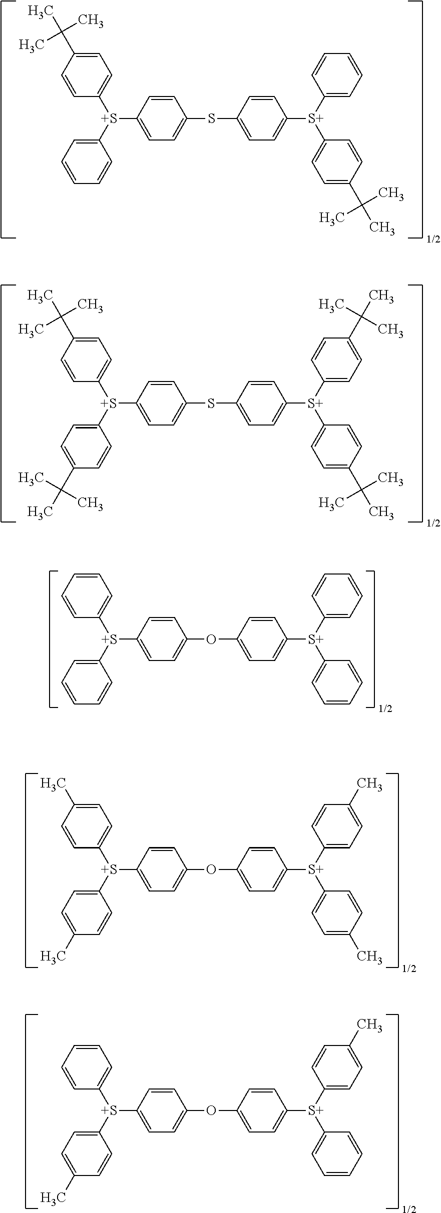

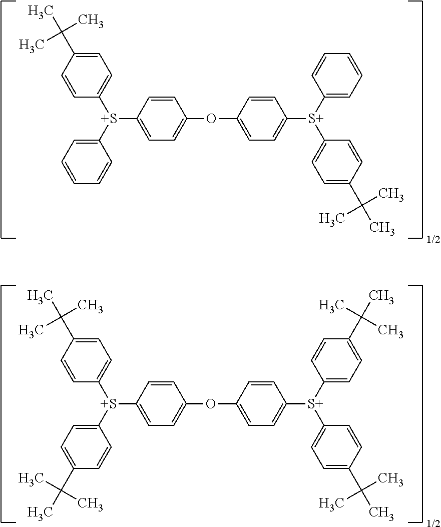

Examples of the salt represented by the formula (B1) include a salt wherein the anion part is any one of the above-mentioned anion part and the cation part is any one of the above-mentioned cation part. The salt represented by the formulae (B1-1) to (B1-17) are preferable, and the salt represented by the formulae (B1-1), (B1-2), (B1-6), (B1-11), (B1-12), (B1-13) and (B1-14) are more preferable.

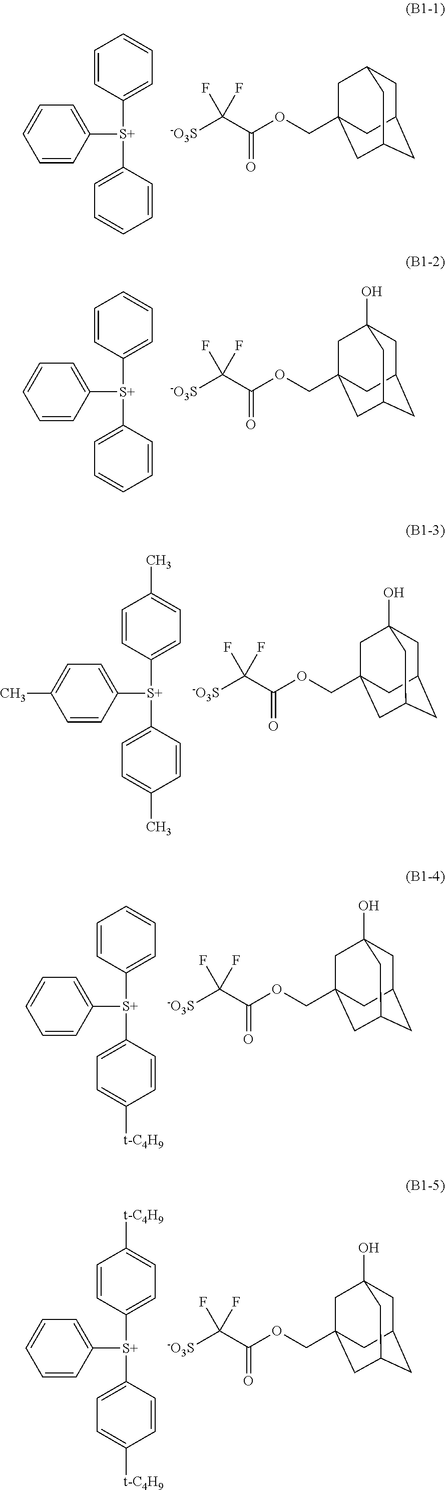

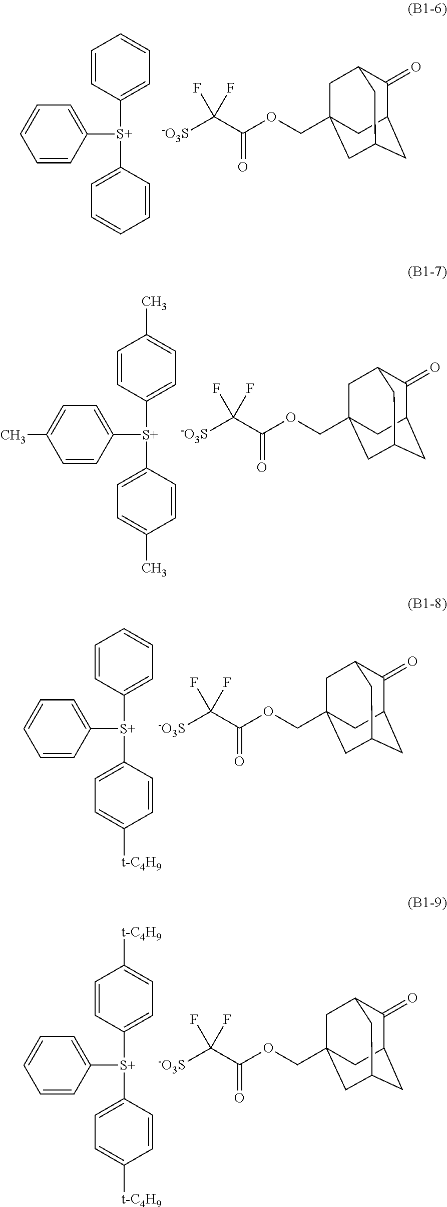

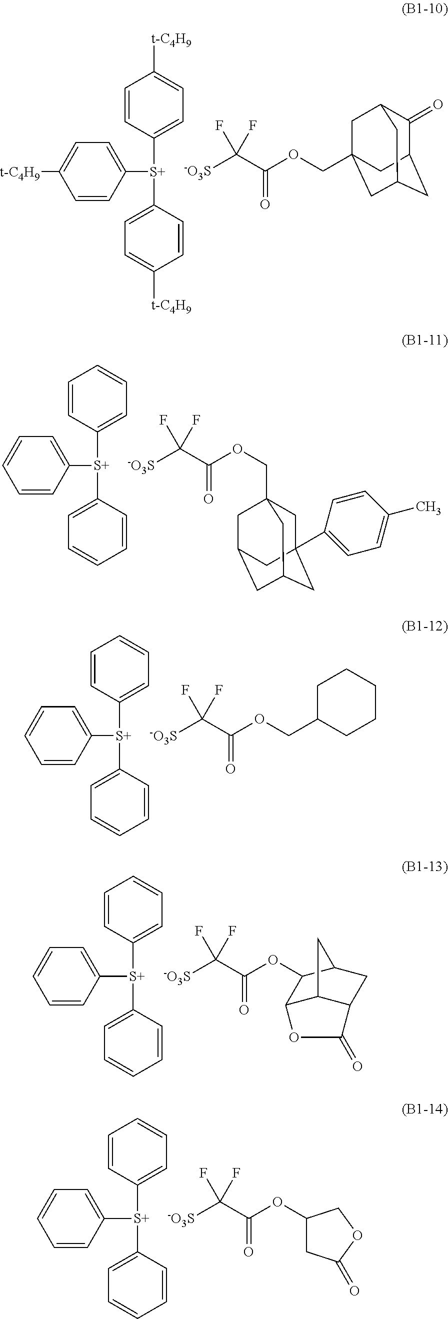

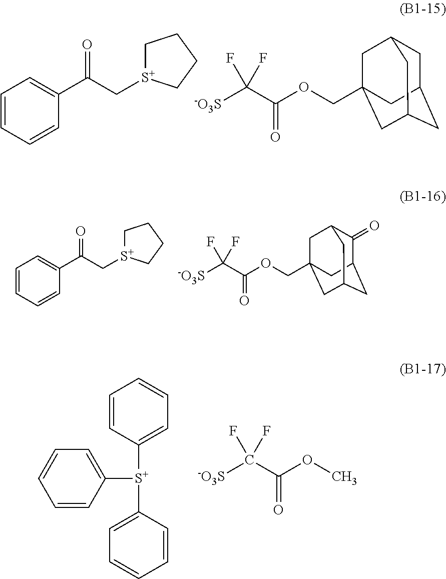

Two or more kinds of the acid generator can be used in combination.

The content of the acid generator is preferably 1 part by weight or more and more preferably 3 parts by weight or more per 100 parts by weight of the resin. The content of the acid generator is preferably 30 parts by weight or less and more preferably 25 parts by weight or less per 100 parts by weight of the resin.

The photoresist composition of the present invention can contain a basic compound as a quencher.

The basic compound is preferably a basic nitrogen-containing organic compound, and examples thereof include an amine compound such as an aliphatic amine and an aromatic amine and an ammonium salt. Examples of the aliphatic amine include a primary amine, a secondary amine and a tertiary amine. Examples of the aromatic amine include an aromatic amine in which aromatic ring has one or more amino groups such as aniline and a heteroaromatic amine such as pyridine. Preferable examples thereof include an aromatic amine represented by the formula (C2):

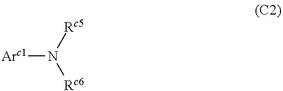

wherein Ar^{c1} represents an aromatic hydrocarbon group, and R^{c5} and R^{c6} each independently represent a hydrogen atom, an aliphatic hydrocarbon group, a saturated cyclic hydrocarbon group or an aromatic hydrocarbon group, and the aliphatic hydrocarbon group, the saturated cyclic hydrocarbon group and the aromatic hydrocarbon group can have one or more substituents selected from the group consisting of a hydroxyl group, an amino group, an amino group having one or two C1-C4 alkyl groups and a C1-C6 alkoxy group.

The aliphatic hydrocarbon group is preferably an alkyl group and the saturated cyclic hydrocarbon group is preferably a cycloalkyl group. The aliphatic hydrocarbon group preferably has 1 to 6 carbon atoms. The saturated cyclic hydrocarbon group preferably has 5 to 10 carbon atoms. The aromatic hydrocarbon group preferably has 6 to 10 carbon atoms.

As the aromatic amine represented by the formula (C2), an amine represented by the formula (C2-1):

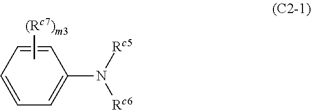

wherein R^{c5} and R^{c6} are the same as defined above, and R^{c7} is independently in each occurrence an aliphatic hydrocarbon group, an alkoxy group, a saturated cyclic hydrocarbon group or an aromatic hydrocarbon group, and the aliphatic hydrocarbon group, the alkoxy group, the saturated cyclic hydrocarbon group and the aromatic hydrocarbon group can have one or more substituents selected from the group consisting of a hydroxyl group, an amino group, an amino group having one or two C1-C4 alkyl groups and a C1-C6 alkoxy group, and m3 represents an integer of 0 to 3, is preferable. The aliphatic hydrocarbon group is preferably an alkyl group and the saturated cyclic hydrocarbon group is preferably a cycloalkyl group. The aliphatic hydrocarbon group preferably has 1 to 6 carbon atoms. The saturated cyclic hydrocarbon group preferably has 5 to 10 carbon atoms. The aromatic hydrocarbon group preferably has 6 to 10 carbon atoms. The alkoxy group preferably has 1 to 6 carbon atoms.

Examples of the aromatic amine represented by the formula (C2) include 1-naphthylamine, 2-naphthylamine, aniline, diisopropylaniline, 2-methylaniline, 3-methylaniline, 4-methylaniline, 4-nitroaniline, N-methylaniline, N,N-dimethylaniline, and diphenylamine, and among them, preferred is diisopropylaniline and more preferred is 2,6-diisopropylaniline.

Other examples of the basic compound include amines represented by the formulae (C3) to (C11):

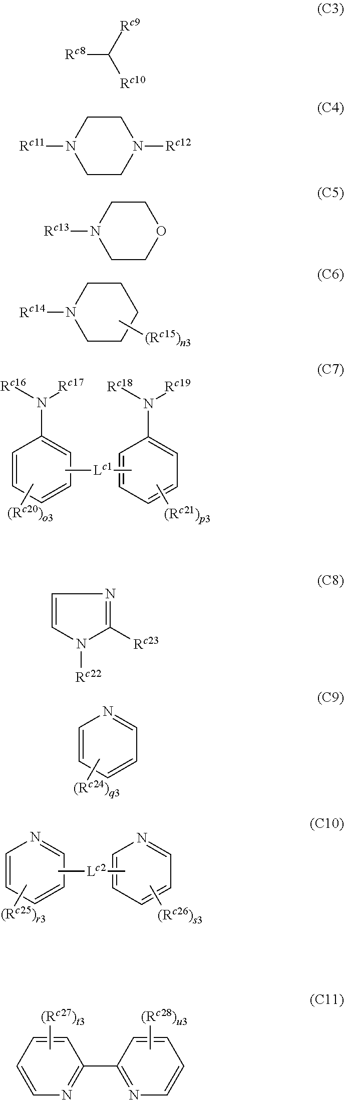

wherein R^{c8}, R^{c20}, R^{c21}, and R^{c23} to R^{c28} each independently represent an aliphatic hydrocarbon group, an alkoxy group, a saturated cyclic hydrocarbon group or an aromatic hydrocarbon group, and the aliphatic hydrocarbon group, the alkoxy group, the saturated cyclic hydrocarbon group and the aromatic hydrocarbon group can have one or more substituents selected from the group consisting of a hydroxyl group, an amino group, an amino group having one or two C1-C4 alkyl groups and a C1-C6 alkoxy group,
R^{c9}, R^{c10}, R^{c11} to R^{c14}, R^{c16} to R^{c19}, and R^{c22} each independently represents a hydrogen atom, an aliphatic hydrocarbon group, a saturated cyclic hydrocarbon group or an aromatic hydrocarbon group, and the aliphatic hydrocarbon group, the saturated cyclic hydrocarbon group and the aromatic hydrocarbon group can have one or more substituents selected from the group consisting of a hydroxyl group, an amino group, an amino group having one or two C1-C4 alkyl groups and a C1-C6 alkoxy group,
R^{c15} is independently in each occurrence an aliphatic hydrocarbon group, a saturated cyclic hydrocarbon group or an alkanoyl group,
L^{c1} and L^{c2} each independently represents a divalent aliphatic hydrocarbon group, —CO—, —C(═NH)—, —C(═NR^{c3})—, —S—, —S—S— or a combination thereof and R^{c3} represents a C1-C4 alkyl group,
O3 to u3 each independently represents an integer of 0 to 3 and n3 represents an integer of 0 to 8.

The aliphatic hydrocarbon group has preferably 1 to 6 carbon atoms, and the saturated cyclic hydrocarbon group has preferably 3 to 6 carbon atoms, and the alkanoyl group has preferably 2 to 6 carbon atoms, and the divalent aliphatic hydrocarbon group has preferably 1 to 6 carbon atoms. The divalent aliphatic hydrocarbon group is preferably an alkylene group.

Examples of the amine represented by the formula (C3) include hexylamine, heptylamine, octylamine, nonylamine, decylamine, dibutylamine, dipentylamine, dihexylamine, diheptylamine, dioctylamine, dinonylamine, didecylamine, triethylamine, trimethylamine, tripropylamine, tributylamine, tripentylamine, trihexylamine, triheptylamine, trioctylamine, trinonylamine, tridecylamine, methyldibutylamine, methyldipentylamine, methyldihexylamine, methyldicyclohexylamine, methyldiheptylamine, methyldioctylamine, methyldinonylamine, methyldidecylamine, ethyldibutylamine, ethydipentylamine, ethyldihexylamine, ethydiheptylamine, ethyldioctylamine, ethyldinonylamine, ethyldidecylamine, dicyclohexylmethylamine, tris[2-(2-methoxyethoxy)ethyl]amine, triisopropanolamine, ethylenediamine, tetramethylenediamine, hexamethylenediamine, 4,4′-diamino-1,2-diphenylethane, 4,4′-diamino-3,3′-dimethyldiphenylmethane and 4,4′-diamino-3,3′-diethyldiphenylmethane.

Examples of the amine represented by the formula (C4) include piperazine. Examples of the amine represented by the formula (C5) include morpholine. Examples of the amine represented by the formula (C6) include piperidine and hindered amine compounds having a piperidine skeleton as disclosed in JP 11-52575 A. Examples of the amine represented by the formula (C7) include 2,2′-methylenebisaniline. Examples of the amine represented by the formula (C8) include imidazole and 4-methylimidazole. Examples of the amine represented by the formula (C9) include pyridine and 4-methylpyridine. Examples of the amine represented by the formula (C10) include di-2-pyridyl ketone, 1,2-di(2-pyridyl)ethane, 1,2-di(4-pyridyl)ethane, 1,3-di(4-pyridyl)propane, 1,2-bis(2-pyridyl)ethene, 1,2-bis(4-pyridyl)ethene, 1,2-di(4-pyridyloxy)ethane, 4,4′-dipyridyl sulfide, 4,4′-dipyridyl disulfide, 2,2′-dipyridylamine and 2,2′-dipicolylamine. Examples of the amine represented by the formula (C11) include bipyridine.

When the basic compound is used, the amount of the basic compound is usually 0.01 to 1 parts by weight per 100 parts by weight of solid component.

The photoresist composition of the present invention usually contains one or more solvents. Examples of the solvent include a glycol ether ester such as ethyl cellosolve acetate, methyl cellosolve acetate and propylene glycol monomethyl ether acetate; a glycol ether such as propylene glycol monomethyl ether; an acyclic ester such as ethyl lactate, butyl acetate, amyl acetate and ethyl pyruvate; a ketone such as acetone, methyl isobutyl ketone, 2-heptanone and cyclohexanone; and a cyclic ester such as γ-butyrolactone.

The amount of the solvent is usually 90% by weight or more, preferably 92% by weight or more preferably 94% by weight or more based on total amount of the photoresist composition of the present invention. The amount of the solvent is usually 99.9% by weight or less and preferably 99% by weight or less based on total amount of the photoresist composition of the present invention.

The photoresist composition of the present invention can contain, if necessary, a small amount of various additives such as a sensitizer, a dissolution inhibitor, other polymers, a surfactant, a stabilizer and a dye as long as the effect of the present invention is not prevented.

The photoresist composition of the present invention is useful for a chemically amplified photoresist composition.

A photoresist pattern can be produced by the following steps (1) to (5):

(1) a step of applying the photoresist composition of the present invention on a substrate,

(2) a step of forming a photoresist film by conducting drying,

(3) a step of exposing the photoresist film to radiation,

(4) a step of baking the exposed photoresist film, and

(5) a step of developing the baked photoresist film with an alkaline developer, thereby forming a photoresist pattern.

The applying of the photoresist composition on a substrate is usually conducted using a conventional apparatus such as spin coater. The photoresist composition is preferably filtrated with filter having 0.2 μm of a pore size before applying. Examples of the substrate include a silicon wafer or a quartz wafer on which a sensor, a circuit, a transistor or the like is formed.

The formation of the photoresist film is usually conducted using a heating apparatus such as hot plate or a decompressor, and the heating temperature is usually 50 to 200° C., and the operation pressure is usually 1 to 1.0*10⁵ Pa.

The photoresist film obtained is exposed to radiation using an exposure system. The exposure is usually conducted through a mask having a pattern corresponding to the desired photoresist pattern. Examples of the exposure source include a light source radiating laser light in a UV-region such as a KrF excimer laser (wavelength: 248 nm), an ArF excimer laser (wavelength: 193 nm) and a F₂ laser (wavelength: 157 nm), and a light source radiating harmonic laser light in a far UV region or a vacuum UV region by wavelength conversion of laser light from a solid laser light source (such as YAG or semiconductor laser).

The temperature of baking of the exposed photoresist film is usually 50 to 200° C., and preferably 70 to 150° C.

The development of the baked photoresist film is usually carried out using a development apparatus. The alkaline developer used may be any one of various alkaline aqueous solution used in the art. Generally, an aqueous solution of tetramethylammonium hydroxide or (2-hydroxyethyl)trimethylammonium hydroxide (commonly known as “choline”) is often used. After development, the photoresist pattern formed is preferably washed with ultrapure water, and the remained water on the photoresist pattern and the substrate is preferably removed.

The photoresist composition of the present invention is suitable for ArF excimer laser lithography, KrF excimer laser lithography, ArF immersion lithography, EUV lithography, EUV immersion lithography, and EB lithography.

### EXAMPLES

The present invention will be described more specifically by Examples, which are not construed to limit the scope of the present invention.

The “%” and “part(s)” used to represent the content of any component and the amount of any material used in the following examples and comparative examples are on a weight basis unless otherwise specifically noted. The weight-average molecular weight of any material used in the following examples is a value found by gel permeation chromatography [HLC-8120GPC Type, Column (Three Columns with guard column): TSKgel Multipore HXL-M, manufactured by TOSOH CORPORATION, Solvent: tetrahydrofuran, Flow rate: 1.0 mL/min., Detector: RI detector, Column temperature: 40° C., Injection volume: 100 μL] using standard polystyrene, manufactured by TOSOH CORPORATION, as a standard reference material. Structures of compounds were determined by NMR (EX-270 Type, manufactured by JEOL LTD.).

### Synthesis Example 1

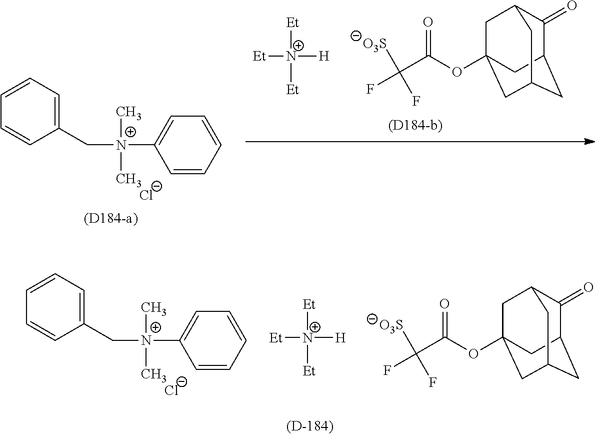

To a solution prepared by mixing 10.0 parts of a compound represented by the formula (D184-b), 50 parts of chloroform and 25 parts of ion-exchanged water, 7.4 parts of a compound represented by the formula (D184-a) was added, and the resultant mixture was stirred at room temperature for 4 hours. The obtained mixture was separated to an organic layer and an aqueous layer. The organic layer was washed with 40 parts of ion-exchanged water and then, concentrated under reduced pressure to obtain 9.8 parts of a compound represented by the formula (D-184). This compound is called as Compound (D-184).

¹H-NMR (dimethyl sulfoxide-d₆): δ (ppm) 7.89-7.77 (2H, m), 7.64-7.53 (3H, m), 7.46-7.24 (3H, m), 7.06-6.97 (2H, m), 5.01 (2H, s), 3.57 (6H, s), 2.56-2.46 (2H, m), 2.37-2.21 (7H, m), 2.03-1.73 (4H, m)

MS (ESI(+)Spectrum): M⁺=212.1 (C₁₅H₁₈N⁺=212.1)

MS (ESI(−)Spectrum): M⁻=323.0 (C₁₂H₁₃F₂O₆S⁻=323.0)

To a solution prepared by mixing 0.05 part of Compound (D-184) with 1 part of dimethyl sulfoxide, ultraviolet rays was irradiated using a UV irradiation apparatus “Spot Cure SP-7” manufactured by USHIO INC. at an intensity of 40 mW for 30 minutes. The solution after irradiation was analyzed with mass spectrometry (Liquid Chromatography: 1100 Type, manufactured by AGILENT TECHNOLOGIES LTD., Mass Spectrometry: LC/MSD Type or LC/MSD TOF Type, manufactured by AGILENT TECHNOLOGIES LTD.). As the result, the generation of N,N-dimethylaniline and Compound (IA-163) was confirmed.

### N,N-dimethylaniline:

MS (ESI(+)Spectrum): [M+H]⁺=122.1 (C₈H₁₁N=121.1)

### Compound (IA-163):

MS (ESI(−)Spectrum): M⁻=323.0 (C₁₂H₁₃F₂O₆S=323.0)

The following components were mixed to prepare a solution, which is called as Solution (Y-184).

| | | |
|---|---|---|
| Resin: Resin A5 | 10 | parts |
| Compound (D): Compound (D-184) | 1.0 | part |
| Solvent: | | |
| propylene glycol monomethyl ether acetate | 60 | parts |
| propylene glycol monomethyl ether | 20 | parts |
| γ-butyrolactone | 3 | parts |

Resin A5 was prepared in the following Resin Synthesis Example 5.

Solution (Y-184) prepared as above was spin-coated over a silicon wafer so that the thickness of the resulting film became 400 nm after prebaking. The silicon wafer thus coated with Solution (Y-184) was prebaked on a direct hotplate at 90° C. for 60 seconds. Using an ArF excimer stepper (“FPA5000-AS3” manufactured by CANON INC., NA=0.75), the whole surface of the wafer formed with the film derived from Solution (Y-184) was subjected to exposure at the exposure amount of 30 mJ/cm².

After the exposure, the wafer was subjected to post-exposure baking at 90° C. for 60 seconds. The film derived from Solution (Y-184) was removed from the wafer by scratching and the obtained film was dissolved in methanol. The obtained methanol solution was analyzed with mass spectrometry (Liquid Chromatography: 1100 Type, manufactured by AGILENT TECHNOLOGIES LTD., Mass Spectrometry: LC/MSD Type or LC/MSD TOF Type, manufactured by AGILENT TECHNOLOGIES LTD.). As the result, the generation of N,N-dimethylaniline and Compound (IA-163) was confirmed.

### N,N-dimethylaniline:

MS (ESI(+)Spectrum): [M+H]⁺=122.1 (C₈H₁₁N=121.1)

### Compound (IA-163):

MS (ESI(−)Spectrum): M⁻=323.0 (C₁₂H₁₃F₂O₆S⁻=323.0)

### Synthesis Example 2

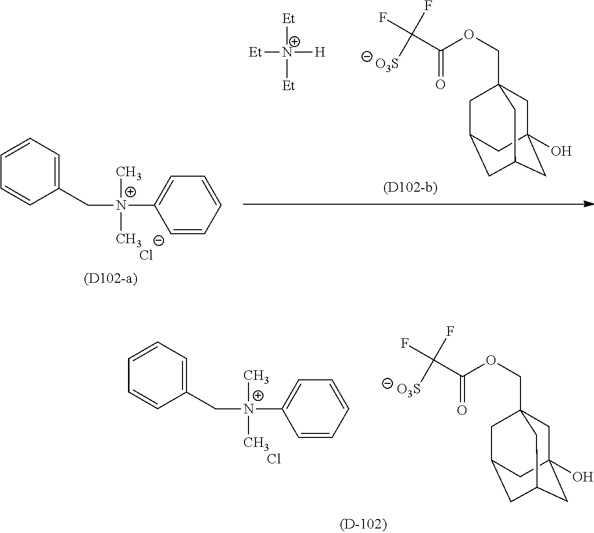

To a solution prepared by mixing 10.0 parts of a compound represented by the formula (D102-b), 50 parts of chloroform and 25 parts of ion-exchanged water, 5.61 parts of a compound represented by the formula (D102-a) was added, and the resultant mixture was stirred at room temperature for 4 hours. The obtained mixture was separated to an organic layer and an aqueous layer. The organic layer was washed with 40 parts of ion-exchanged water and then, concentrated under reduced pressure to obtain 9.9 parts of a compound represented by the formula (D-102). This compound is called as Compound (D-102).

¹H-NMR (dimethyl sulfoxide-d₆): δ (ppm) 7.89-7.77 (2H, m), 7.64-7.53 (3H, m), 7.46-7.24 (3H, m), 7.06-6.97 (2H, m), 5.01 (2H, s), 4.42 (1H, s), 3.84 (2H, s), 3.57 (6H, s), 2.12-2.01 (2H, m), 1.57-1.28 (12H, m)

MS (ESI(+)Spectrum): M⁺=212.1 (C₁₅H₁₈N⁺=212.1)

MS (ESI(−)Spectrum): M⁻=339.1 (C₁₃H₁₇F₂O₆S⁻=339.1)

To a solution prepared by mixing 0.05 part of Compound (D-102) with 1 part of dimethyl sulfoxide, ultraviolet rays was irradiated using a UV irradiation apparatus “Spot Cure SP-7” manufactured by USHIO INC. at an intensity of 40 mW for 30 minutes. The solution after irradiation was analyzed with mass spectrometry (Liquid Chromatography: 1100 Type, manufactured by AGILENT TECHNOLOGIES LTD., Mass Spectrometry: LC/MSD Type or LC/MSD TOF Type, manufactured by AGILENT TECHNOLOGIES LTD.). As the result, the generation of N,N-dimethylaniline and Compound (IA-94) was confirmed.

### N,N-dimethylaniline:

MS (ESI(+)Spectrum): [M+H]⁺=122.1 (C₈H₁₁N=121.1)

### Compound (IA-94):

MS (ESI(−)Spectrum): M⁻=339.1 (C₁₃H₁₇F₂O₆S⁻=339.1)

The following components were mixed to prepare a solution, which is called as Solution (Y-102).

| | | |
|---|---|---|
| Resin: Resin A5 | 10 | parts |
| Compound (D): Compound (D-102) | 1.0 | part |
| Solvent: | | |
| propylene glycol monomethyl ether acetate | 60 | parts |
| propylene glycol monomethyl ether | 20 | parts |
| γ-butyrolactone | 3 | parts |

Resin A5 was prepared in the following Resin Synthesis Example 5.

Solution (Y-102) prepared as above was spin-coated over a silicon wafer so that the thickness of the resulting film became 400 nm after prebaking. The silicon wafer thus coated with Solution (Y-148) was prebaked on a direct hotplate at 90° C. for 60 seconds. Using an ArF excimer stepper (“FPA5000-AS3” manufactured by CANON INC., NA=0.75), the whole surface of the wafer formed with the film derived from Solution (Y-148) was subjected to exposure at the exposure amount of 30 mJ/cm².

After the exposure, the wafer was subjected to post-exposure baking at 90° C. for 60 seconds. The film derived from Solution (Y-148) was removed from the wafer by scratching and the obtained film was dissolved in methanol. The obtained methanol solution was analyzed with mass spectrometry (Liquid Chromatography: 1100 Type, manufactured by AGILENT TECHNOLOGIES LTD., Mass Spectrometry: LC/MSD Type or LC/MSD TOF Type, manufactured by AGILENT TECHNOLOGIES LTD.). As the result, the generation of N,N-dimethylaniline and Compound (IA-94) was confirmed.

### N,N-dimethylaniline:

MS (ESI(+)Spectrum): [M+H]⁺=122.1 (C₈H₁₁N=121.1)

### Compound (IA-94):

MS (ESI(−)Spectrum): M⁻=339.1 (C₁₃H₁₇F₂O₆S⁻=339.1)

### Synthesis Example 3

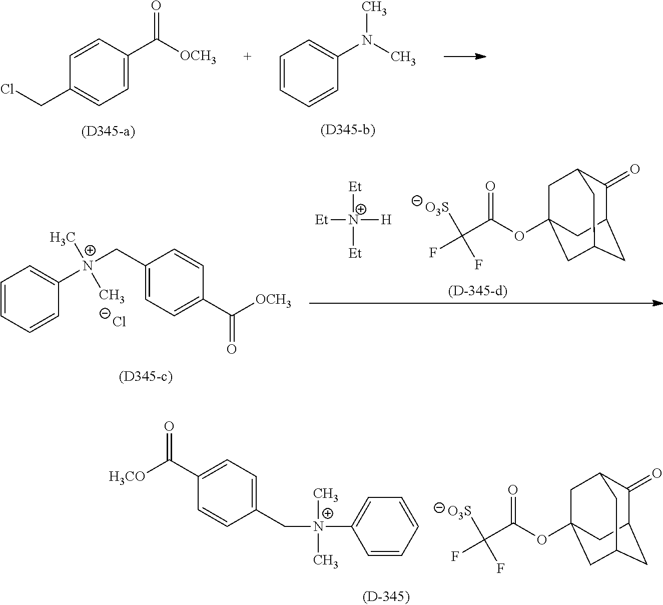

To a solution prepared by mixing 7.0 parts of a compound represented by the formula (D345-a), 35 parts of acetonitrile and 5 parts of ion-exchanged water, 4.6 parts of a compound represented by the formula (D345-b) was added, and the resultant mixture was stirred for 20 hours under reflux. The obtained mixture was mixed with 70 parts of methyl tert-butyl ether and then, the obtained mixture was stirred at room temperature for 1 hour. The precipitate was isolated by filtration to obtain 7.6 parts of a compound represented by the formula (D345-c).

¹H-NMR (dimethyl sulfoxide-d₆): δ (ppm) 7.96-7.79 (4H, m), 7.64-7.52 (3H, m), 7.21 (2H, d, J=8.2 Hz), 5.26 (2H, s), 3.82 (3H, s), 3.66 (6H, s)

To a solution prepared by mixing 5.0 parts of a compound represented by the formula (D345-c), 40 parts of chloroform and 10 parts of ion-exchanged water, 3.6 parts of a compound represented by the formula (D345-d) was added, and the resultant mixture was stirred at room temperature for 20 hours. The obtained mixture was separated to an organic layer and an aqueous layer. The organic layer was washed with 15 parts of ion-exchanged water and then, concentrated under reduced pressure to obtain 5.9 parts of a compound represented by the formula (D-345). This compound is called as Compound (D-345).

¹H-NMR (dimethyl sulfoxide-d₆): δ (ppm) 7.91-7.78 (4H, m), 7.65-7.55 (3H, m), 7.17 (2H, d, J=8.2 Hz), 5.08 (2H, s), 3.83 (3H, s), 3.59 (6H, s), 2.56-2.44 (3H, m), 2.33-2.20 (6H, m), 2.04-1.91 (2H, m), 1.88-1.76 (2H, m)

MS (ESI(+)Spectrum): M⁺=270.1 (C₁₇H₂₀NO₂⁺=270.1)

MS (ESI(−)Spectrum): M⁻=323.0 (C₁₂H₁₃F₂O₆S⁻=323.0)

To a solution prepared by mixing 0.05 part of Compound (D-345) with 1 part of dimethyl sulfoxide, ultraviolet rays was irradiated using a UV irradiation apparatus “Spot Cure SP-7” manufactured by USHIO INC. at an intensity of 40 mW for 30 minutes. The solution after irradiation was analyzed with mass spectrometry (Liquid Chromatography: 1100 Type, manufactured by AGILENT TECHNOLOGIES LTD., Mass Spectrometry: LC/MSD Type or LC/MSD TOF Type, manufactured by AGILENT TECHNOLOGIES LTD.). As the result, the generation of N,N-dimethylaniline and Compound (IA-163) was confirmed.

### N,N-dimethylaniline:

MS (ESI(+)Spectrum): [M+H]⁺=122.1 (C₈H₁₁N=121.1)

### Compound (IA-163):

MS (ESI(−)Spectrum): M⁻=323.0 (C₁₂H₁₃F₂O₆S⁻=323.0)

The following components were mixed to prepare a solution, which is called as Solution (Y-345).

| | | |
|---|---|---|
| Resin: Resin A5 | 10 | parts |
| Compound (D): Compound (D-345) | 1.0 | part |
| Solvent: | | |
| propylene glycol monomethyl ether acetate | 60 | parts |
| propylene glycol monomethyl ether | 20 | parts |
| γ-butyrolactone | 3 | parts |

Resin A5 was prepared in the following Resin Synthesis Example 5.

Solution (Y-345) prepared as above was spin-coated over a silicon wafer so that the thickness of the resulting film became 400 nm after prebaking. The silicon wafer thus coated with Solution (Y-345) was prebaked on a direct hotplate at 90° C. for 60 seconds. Using an ArF excimer stepper (“FPA5000-AS3” manufactured by CANON INC., NA=0.75), the whole surface of the wafer formed with the film derived from Solution (Y-345) was subjected to exposure at the exposure amount of 30 mJ/cm².

After the exposure, the wafer was subjected to post-exposure baking at 90° C. for 60 seconds. The film derived from Solution (Y-345) was removed from the wafer by scratching and the obtained film was dissolved in methanol. The obtained methanol solution was analyzed with mass spectrometry (Liquid Chromatography: 1100 Type, manufactured by AGILENT TECHNOLOGIES LTD., Mass Spectrometry: LC/MSD Type or LC/MSD TOF Type, manufactured by AGILENT TECHNOLOGIES LTD.). As the result, the generation of N,N-dimethylaniline and Compound (IA-163) was confirmed.

### N,N-dimethylaniline:

MS (ESI(+)Spectrum): [M+H]⁺=122.1 (C₈H₁₁N=121.1)

### Compound (IA-163):

MS (ESI(−)Spectrum): M⁻=323.0 (C₁₂H₁₃F₂O₆S=323.0)

Monomers used in the following Resin Synthetic Examples are following Monomer (M-1), Monomer (M-2), Monomer (M-3), Monomer (M-4), Monomer (M-5) and Monomer (M-6).

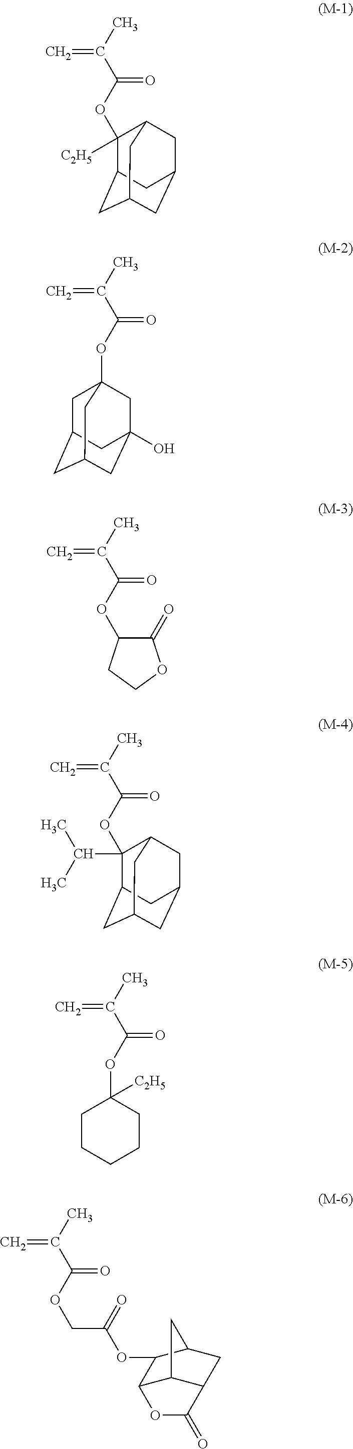

The molar ratio of the structural units in the resin was calculated based on the amount of the unreacted monomers in the reaction mixture, which was measured by liquid chromatography analysis.

### Resin Synthesis Example 1

Into a flask, 15.00 parts of Monomer (M-1), 4.89 parts of Monomer (M-2), 11.12 parts of Monomer (M-6) and 8.81 parts of Monomer (M-3) (molar ratio: Monomer (M-1)/Monomer (M-2)/Monomer (M-6)/Monomer (M-3)=35/12/23/30) were charged, and 1,4-dioxane of which amount was 1.5 times part based on total parts of all monomers was added thereto to prepare a solution. To the solution, 2,2′-azobisisobutyronitrile as an initiator in a ratio of 1 mol % based on all monomer molar amount and 2,2′-azobis(2,4-dimethylvaleronitrile) as an initiator in a ratio of 3 mol % based on all monomer molar amount were added, and the obtained mixture was heated at 77° C. for about 5 hours. The reaction mixture obtained was poured into a large amount of a mixture of methanol and water to cause precipitation. The precipitate was isolated and mixed with a large amount of a mixture of methanol and water followed by filtration. This operation wherein the precipitate was isolated and mixed with a large amount of a mixture of methanol and water followed by filtration was repeated three times for purification. As the result, a resin having a weight-average molecular weight of about 8.1×10³ and a dispersion degree (Mw/Mn) of 1.79 was obtained in a yield of 78%. This resin had the structural units represented by the followings. This resin is called as Resin A1.

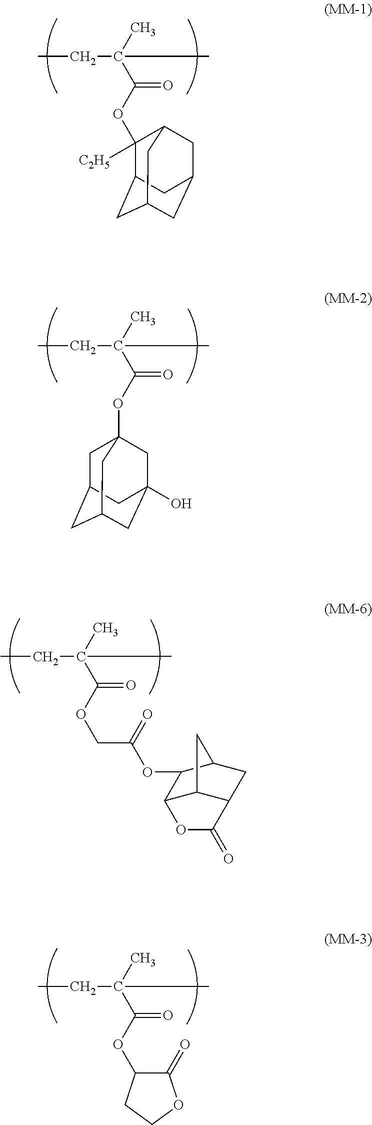

The molar ratio of the structural units represented by the formulae (MM-1), (MM-2), (MM-6) and (MM-3) ((MM-1)/(MM-2)/(MM-6)/(MM-3)) was 28/13/25/34.

### Resin Synthesis Example 2

Into a flask equipped with a condenser and a thermometer, 72.77 parts of 72.77 parts of 1,4-dioxane was charged, and a nitrogen gas was blown into it for 30 minutes. After heating it up to 75° C. under nitrogen, a solution prepared by mixing 76.30 parts of Monomer (M-4), 11.42 parts of Monomer (M-5), 11.74 parts of Monomer (M-2), 52.16 parts of Monomer (M-6), 0.96 parts of 2,2′-azobisisobutyronitrile, 4.33 parts of 2,2′-azobis(2,4-dimethylvaleronitrile) and 109.16 parts of 1,4-dioxane was added dropwise thereto over 2 hour at 75° C. The resultant mixture was stirred for 5 hours at 75° C. After cooling the reaction mixture down to room temperature, the reaction mixture was diluted with 212.26 parts of 1,4-dioxane and the resultant solution was poured into a mixture of 536 parts of methanol and 394 parts of water to cause precipitation. The precipitate was isolated and mixed with 985 parts of methanol. The resultant mixture was stirred followed by filtrating to obtain the precipitate. The operation wherein the precipitate was mixed with 985 parts of methanol and the resultant mixture was stirred followed by filtrating to obtain the precipitate was repeated three times for purification. The obtained precipitate was dried under reduced pressure to obtain 112 parts of a resin having a weight-average molecular weight (Mw) of 7.4×10³ and a dispersion degree (Mw/Mn) of 1.83 in a yield of 74%. This resin had the structural units represented by the followings. This is called as Resin A2.

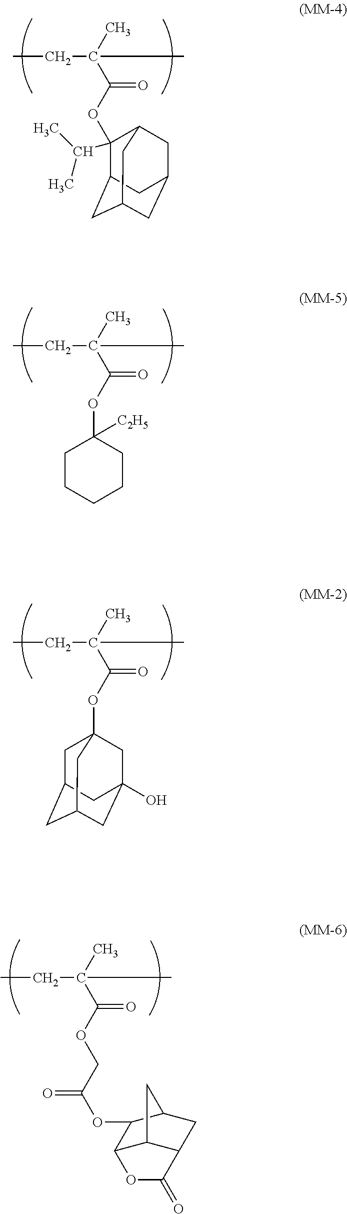

The molar ratio of the structural units represented by the formulae (MM-4), (MM-5), (MM-2) and (MM-6) ((MM-4)/(MM-5)/(MM-2)/(MM-6)) was 40/10/10/40.

### Resin Synthesis Example 3

A solution prepared by dissolving 59.6 parts of 2-ethyl-2-adamantyl methacrylate and 90.8 parts of p-acetoxystyrene in 279 parts of isopropanol was heated up to 75° C. To a solution, a solution prepared by dissolving 11.05 parts of dimethyl 2,2′-azobis(2-methylpropinonate) in 22.11 parts of isopropanol was added dropwise, and the resultant mixture was stirred for 0.3 hour at 75° C. and then, was further stirred for 12 hours under reflux. The obtained reaction mixture was diluted with acetone. The resultant mixture was poured into methanol to cause precipitation. The precipitate was collected by filtration to obtain 250 parts of a crude resin derived from 2-ethyl-2-adamantyl methacrylate and p-acetoxystyrene (molar ratio: structural unit derived from 2-ethyl-2-adamantyl methacrylate/structural unit derived from p-acetoxystyrene=30/70).

Into a flask, 250 parts of the crude resin, 10.8 parts of 4-dimethylaminopyridine and 239 parts of methanol were charged, and the resultant mixture was refluxed for 20 hours. The obtained mixture was cooled and then, was neutralized with 8.0 parts of glacial acetic acid. The obtained mixture was poured into water to cause precipitation. The precipitate was collected by filtration and dissolved in acetone. The obtained solution was poured into water to cause precipitation, and the precipitate was collected by filtration. This operation was repeated three times followed by drying to obtain 102.8 parts of a resin having a weight-average molecular weight of about 8.2×10³ and a dispersion degree (Mw/Mn) of 1.68. This resin had the structural units represented by the followings. This resin is called as Resin A3.

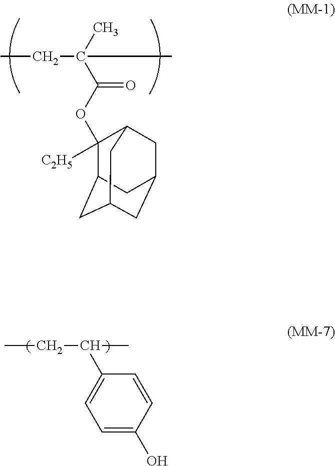

### Resin Synthesis Example 4

A solution prepared by dissolving 14.06 parts of 2-methyl-2-adamantyl methacrylate, 27.26 parts of p-(ethoxyethyl)styrene and 4.73 parts of 3-hydroxy-1-adamantyl methacrylate in 62.78 parts of 1,4-dioxane was heated up to 87° C. To a solution, 2.96 parts of 2,2′-azobisisobutyronitrile was added, and the resultant mixture was stirred for 6 hours at 87° C. The obtained reaction mixture was cooled and then, was poured into a mixture of 389.89 parts of methanol and 163.24 parts of ion-exchanged water to cause precipitation. The precipitate was collected by filtration. The obtained precipitate and 4.10 parts of 4-dimethylaminopyridine were added to methanol of which amount was the same as that of the obtained precipitate, and the resultant mixture was refluxed for 15 hours. The obtained mixture was cooled and then, was neutralized with 2.16 parts of glacial acetic acid. The obtained mixture was poured into a large amount of water to cause precipitation. The precipitate was collected by filtration and dissolved in acetone. The obtained solution was poured into a large amount of water to cause precipitation, and the precipitate was collected by filtration. This operation was repeated three times followed by drying to obtain 32.15 parts of a resin having a weight-average molecular weight of about 4.8×10³ and a dispersion degree (Mw/Mn) of 1.53. This resin had the structural units represented by the followings. This resin is called as Resin A4.

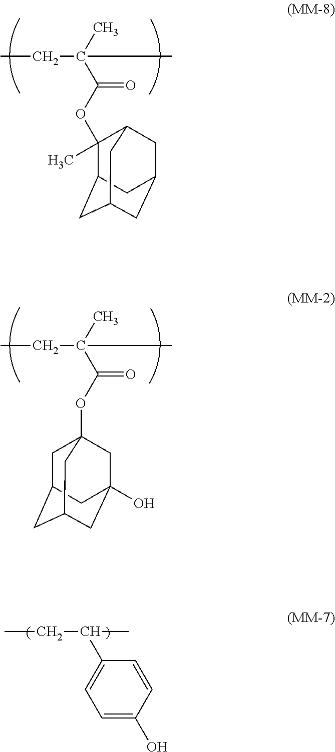

The molar ratio of the structural units represented by the formulae (MM-8), (MM-7) and (MM-2) ((MM-8)/(MM-7)/(MM-2)) was 30/60/10.

Monomers used in the following Resin Synthetic Example 5 are following Monomer (M-2), Monomer (M-6) and Monomer (M-9).

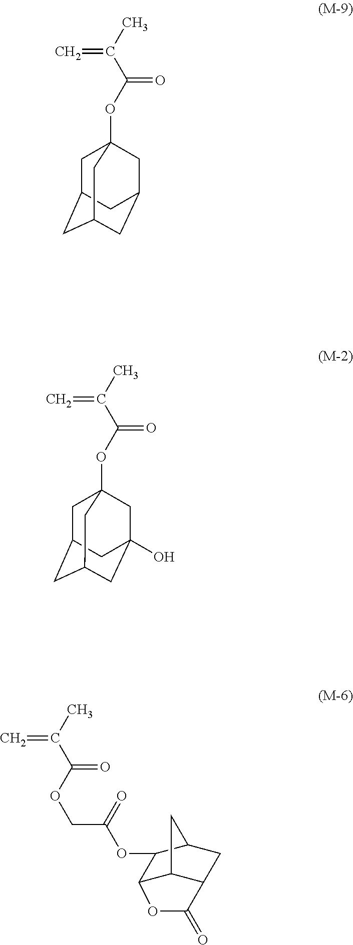

The molar ratio of the structural units in the resin was calculated based on the amount of the unreacted monomers in the reaction mixture, which was measured by liquid chromatography analysis.

### Resin Synthesis Example 5

Into a four-necked flask equipped with a condenser, a stirrer and a thermometer, 24.0 parts of 1,4-dioxane was added to heat up to 72° C. To this, a solution prepared by dissolving 20.00 parts of Monomer (M-9), 4.29 parts of Monomer (M-2), 15.73 parts of Monomer (M-6), 0.27 part of 2,2′-azobisisobutyronitrile and 1.23 parts of 2,2′-azobis(2,4-dimethylvaleronitrile) in 36.02 parts of 1,4-dioxane was added dropwise over 1 hour at 72° C. The obtained mixture was stirred at 72° C. for 5 hours. The reaction mixture obtained was poured into 520 parts of methanol to cause precipitation. The precipitate was isolated and dried at 40° C. under reduced pressure. As the result, 35.42 parts of a resin having a weight-average molecular weight of 1.0×10⁴ and a dispersion degree (Mw/Mn) of 1.7 was obtained. This resin had the structural units represented by the followings. This resin is called as Resin A5.

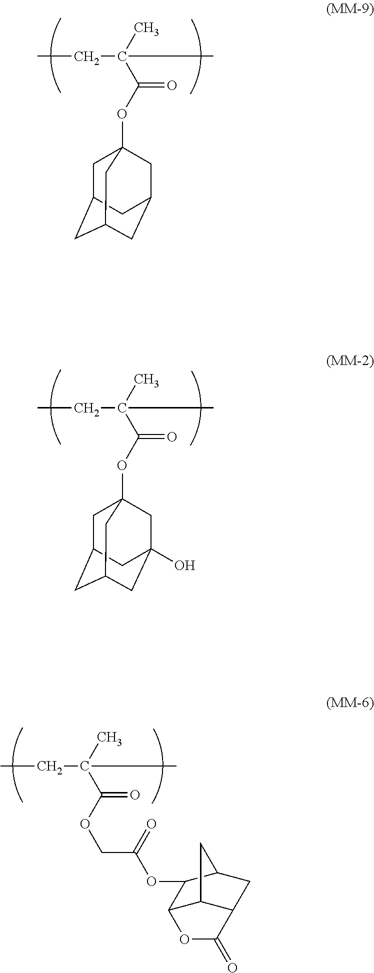

The molar ratio of the structural units represented by the formulae (MM-9), (MM-2) and (MM-6) ((MM-9)/(MM-2)/(MM-6)) was 55/11/34.

### Examples 1 to 4 and Reference Example 1

### Resin

### Resin A1

### <Acid Generator>

### B1:

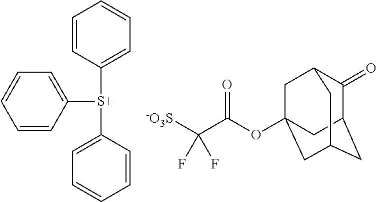

### <Compound (D)>

### D-184: Compound (D-184)

### D-102: Compound (D-102)

### D-345: Compound (D-345)

### <Quencher>

C1: 2,6-diisopropylaniline

### <Solvent>

### S1:

| | | |
|---|---|---|
| propylene glycol monomethyl ether acetate | 115 | parts |
| propylene glycol monomethyl ether | 20 | parts |
| 2-heptanone | 25 | parts |
| γ-butyrolactone | 3 | parts |

The following components were mixed and dissolved to prepare photoresist compositions.

Resin (kind and amount are described in Table 13)

Acid generator (kind and amount are described in Table 13)

Compound (D) (kind and amount are described in Table 13)

Quencher (kind and amount are described in Table 13)

Solvent S1

TABLE 13

| | Resin | Acid generator | Compound (D) | Quencher |
|---|---|---|---|---|
| | (kind/amount | (kind/amount | (kind/amount | (kind/amount |
| Ex. No. | (part)) | (part)) | (part)) | (part)) |
| Ex. 1 | A1/10 | B1/0.50 | D-184/0.2 | C1/0.065 |
| Ex. 2 | A1/10 | B1/0.50 | D-184/0.4 | C1/0.065 |
| Ex. 3 | A1/10 | B1/0.50 | D-102/0.2 | C1/0.065 |
| Ex. 4 | A1/10 | B1/0.50 | D-345/0.2 | C1/0.065 |
| Ref. Ex. 1 | A1/10 | B1/0.50 | — | C1/0.0325 |

Silicon wafers were each coated with “ARC-29”, which is an organic anti-reflective coating composition available from Nissan Chemical Industries, Ltd., and then baked under the conditions: 205° C., 60 seconds, to form a 780 Å-thick organic anti-reflective coating. Each of the photoresist compositions prepared as above was spin-coated over the anti-reflective coating so that the thickness of the resulting film became 0.16 μm after drying. The silicon wafers thus coated with the respective photoresist compositions were each prebaked on a direct hotplate at 100° C. for 60 seconds. Using an ArF excimer stepper (“FPA5000-AS3” manufactured by CANON INC., NA=0.75, 2/3 Annular), each wafer thus formed with the respective photoresist film was subjected to line and space pattern exposure, with the exposure quantity being varied stepwise.

After the exposure, each wafer was subjected to post-exposure baking on a hotplate at 105° C. for 60 seconds and then to paddle development for 60 seconds with an aqueous solution of 2.38 wt % tetramethylammonium hydroxide.

Each of line and space patterns developed on the organic anti-reflective coating substrate after the development was observed with a scanning electron microscope, the results of which are shown in Table 14.

Effective Sensitivity (ES): It was expressed as the amount of exposure that the line pattern and the space pattern of 100 nm become 1:1 after exposure and development.

Line Width Roughness (LWR): The line widths of the line and space pattern at the exposure amount of ES were measured and the values of 3σ thereof were calculated based on the results of the measurement and shown in Table 11. The value of 3σ is one of index showing a variability of the line width and the smaller the value of 3σ is, the better LWR is. When the value of 3σ is 10 nm or less, LWR is good, and its evaluation is marked by “◯”, and when the value of 3σ is more than 10 nm, LWR is bad, and its evaluation is marked by “X”.

TABLE 14

| Ex. No. | LWR |
|---|---|
| Ex. 1 | ◯ |
| Ex. 2 | ◯ |
| Ex. 3 | ◯ |
| Ex. 4 | ◯ |
| Ref. Ex. 1 | X |

### Examples 5 to 6 and Comparative Example 2

### Resin

### Resin A3, A4

### <Acid Generator>

### B2:

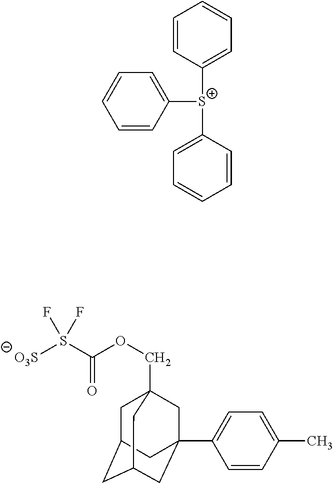

### <Compound (D)>

### D-184: Compound (D-184)

### D-345: Compound (D-345)

### <Quencher>

C1: 2,6-diisopropylaniline

### <Solvent>

### S2:

| | |
|---|---|
| propylene glycol monomethyl ether acetate | 150 parts |
| propylene glycol monomethyl ether | 420 parts |
| γ-butyrolactone | 5 parts |

The following components were mixed and dissolved to prepare photoresist compositions.

Resin (kind and amount are described in Table 15)

Acid generator (kind and amount are described in Table 15)

Compound (D) (kind and amount are described in Table 15)

Quencher (kind and amount are described in Table 15)

Solvent S2

TABLE 15

| | Resin | Acid generator | Compound (D) | Quencher |
|---|---|---|---|---|
| | (kind/amount | (kind/amount | (kind/amount | (kind/amount |
| Ex. No. | (part)) | (part)) | (part)) | (part)) |
| Ex. 5 | A4/10 | B2/2.0 | D-184/0.79 | C1/0.1 |
| Ex. 6 | A4/10 | B2/2.0 | D-345/0.88 | C1/0.1 |
| Ref. Ex. 2 | A3/10 | B2/1.5 | — | C1/0.1 |

Silicon wafers were each contacted with hexamethyldisilazane at 90° C. for 60 seconds on a direct hotplate and each of the photoresist compositions prepared as above was spin-coated over the silicon wafer to give a film thickness after drying of 0.06 μm. After application of each of the photoresist compositions, the silicon wafers thus coated with the respective resist compositions were each prebaked on a direct hotplate at 110° C. for 60 seconds. Using a writing electron beam lithography system (“HL-800D” manufactured by Hitachi, Ltd., 50 KeV), each wafer on which the respective resist film had been thus formed was exposed to a line and space pattern, while changing stepwise the exposure quantity.

After the exposure, each wafer was subjected to post-exposure baking on a hotplate at 110° C. for 60 seconds and then to paddle development with an aqueous solution of 2.38% by weight tetramethylammonium hydroxide for 60 seconds.

Each of a photoresist pattern developed on the silicon substrate after the development was observed with a scanning electron microscope, and the results of which are shown in Table 16.

Resolution: The amount of exposure that each photoresist pattern became 1:1 line and space pattern was as effective sensitivity. When line and space pattern having 50 nm or less of the line width was developed at effective sensitivity, resolution is good and its evaluation is marked by “◯”, and when line and space pattern having 50 nm of the line width was not developed at effective sensitivity, resolution is bad and its evaluation is marked by “X”.

TABLE 16

| Ex. No. | Resolution |
|---|---|
| Ex. 5 | ◯ |
| Ex. 6 | ◯ |
| Com. Ex. 2 | X |

### Examples 7 and 8 and Reference Example 3

### Resin

### Resin A1

### <Acid Generator>

### B3:

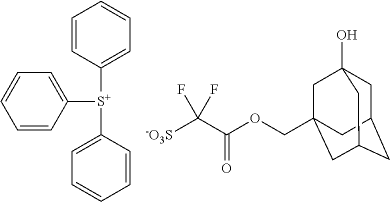

### <Compound (D)>

### D-184: Compound (D-184)

### D-345: Compound (D-345)

### <Quencher>

C1: 2,6-diisopropylaniline

### <Solvent>

### S3:

| | | |
|---|---|---|
| propylene glycol monomethyl ether acetate | 180 | parts |
| propylene glycol monomethyl ether | 20 | parts |
| 2-heptanone | 10 | parts |
| γ-butyrolactone | 3 | parts |

The following components were mixed and dissolved to prepare photoresist compositions.

Resin (kind and amount are described in Table 17)

Acid generator (kind and amount are described in Table 17)

Compound (D) (kind and amount are described in Table 17)

Quencher (kind and amount are described in Table 17)

Solvent S3

TABLE 17

| | Resin | Acid generator | Compound (D) | Quencher |
|---|---|---|---|---|
| | (kind/amount | (kind/amount | (kind/amount | (kind/amount |
| Ex. No. | (part)) | (part)) | (part)) | (part)) |
| Ex. 7 | A1/10 | B1/0.95 | D-184/0.2 | C1/0.012 |
| Ex. 8 | A1/10 | B1/0.95 | D-345/0.2 | C1/0.012 |
| Ref. Ex. 3 | A1/10 | B1/0.95 | — | C1/0.012 |

Silicon wafers were each coated with “ARC-29SR”, which is an organic anti-reflective coating composition available from Nissan Chemical Industries, Ltd., and then baked under the conditions: 205° C., 60 seconds, to form a 93 nm-thick organic anti-reflective coating. Each of the photoresist compositions prepared as above was spin-coated over the anti-reflective coating so that the thickness of the resulting film became 100 nm after prebaking. The silicon wafers thus coated with the respective photoresist compositions were each prebaked on a direct hotplate at 90° C. for 60 seconds. Using an ArF excimer stepper (“XT: 1900Gi” manufactured by ASML, NA=1.35, 3/4 Annular, σ OUTER=0.9, σ INNER=0.675), each wafer thus formed with the respective resist film was subjected to contact hole pattern exposure with the exposure quantity being varied stepwise.

After the immersion exposure, each wafer was subjected to post-exposure baking on a hotplate at 90° C. for 60 seconds and then to paddle development for 60 seconds with an aqueous solution of 2.38 wt % tetramethylammonium hydroxide.

Effective Sensitivity (ES): It is expressed as the amount of exposure that the a contact hole pattern having a hole diameter of 50 nm is obtained after exposure through a contact hole pattern mask having a diameter of 70 nm and development.

CD uniformity (CDU): The photoresist pattern at the exposure amount of the effective sensitivity was observed with a scanning electron microscope. The hole-diameter of the hole of the contact hole pattern was twenty four times measured and its average diameter was calculated. The average diameters of 496 holes on the same wafer were respectively measured. When population was the average diameters of 496 holes, the standard deviation was calculated. When the standard deviation is less than 2.20 nm, CDU is good and its evaluation is marked by “◯”, when the standard deviation is 2.20 nm or more and less than 2.50 nm, CDU is usual and its evaluation is marked by “Δ”, and when the standard deviation is 2.50 nm or more, CDU is usual and its evaluation is marked by “X”. The smaller the standard deviation is, the better pattern profile is.

TABLE 18

| Ex. No. | CDU |
|---|---|
| Ex. 7 | ◯ |
| Ex. 8 | ◯ |
| Ref. Ex. 3 | Δ |

The present photoresist composition provides a good resist pattern having good resolution and good pattern profile such as Line width roughness and CD uniformity, and is especially suitable for ArF excimer laser lithography, EB lithography and EUV lithography.

What is claimed is:

1. A photoresist composition comprising a compound capable of generating an acid and a base by irradiation, a resin having an acid-labile group and being insoluble or poorly soluble in an aqueous alkali solution but becoming soluble in an aqueous alkali solution by the action of an acid, and an acid generator.

2. The photoresist composition according to claim 1, wherein the base generated by irradiation to the compound capable of generating an acid and a base by irradiation is a base represented by the formula (IB): 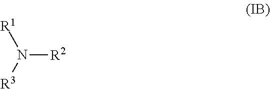
wherein R¹, R² and R³ each independently represent C1-C12 hydrocarbon group which can have one or more substituents, and two or three selected from the group consisting of R¹, R² and R³ can be bonded each other to form a ring together with the nitrogen atom to which they are bonded.

3. The photoresist composition according to claim 1, wherein the acid generated by irradiation to the compound capable of generating an acid and a base by irradiation is an acid represented by the formula (IA): 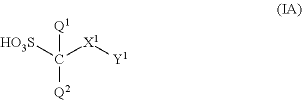
wherein Q¹ and Q² each independently represent a fluorine atom or a C1-C6 perfluoroalkyl group, X¹ represents a single bond or a C1-C17 divalent saturated hydrocarbon group in which one or more —CH₂— can be replaced by —O— or —CO—,
Y¹ represents a C1-C36 aliphatic hydrocarbon group which can have one or more substituents, a C3-C36 saturated cyclic hydrocarbon group which can have one or more substituents, or a C6-C36 aromatic hydrocarbon group which can have one or more substituents, and one or more —CH₂— in the aliphatic hydrocarbon group, the saturated cyclic hydrocarbon group and the aromatic hydrocarbon group can be replaced by —O— or —CO—.

4. The photoresist composition according to claim 1, wherein the compound capable of generating an acid and a base by irradiation is a salt represented by the formula (ID): 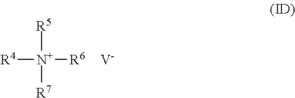
wherein R⁴, R⁵ and R⁶ each independently represent C1-C12 hydrocarbon group which can have one or more substituents, and two or three selected from the group consisting of R⁴, R⁵ and R⁶ can be bonded each other to form a ring together with the nitrogen atom to which they are bonded, R⁷ represents an organic group having an aromatic hydrocarbon group, and V⁻ represents an organic counter anion.

5. The photoresist composition according to claim 4, wherein R⁷ is a benzyl group which can have one or more substituents or a phenylethyl group which can have one or more substituents.

6. A salt represented by the formula (I-1): 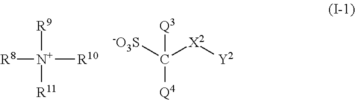
wherein R⁸, R⁹ and R¹⁰ each independently represent C1-C12 hydrocarbon group which can have one or more substituents, and two or three selected from the group consisting of R⁸, R⁹ and R¹⁰ can be bonded each other to form a ring together with the nitrogen atom to which they are bonded, R¹¹ represents a benzyl group which can have one or more substituents or a phenylethyl group which can have one or more substituents, Q³ and Q⁴ each independently represent a fluorine atom or a C1-C6 perfluoroalkyl group, X² represents a single bond or a C1-C17 divalent saturated hydrocarbon group in which one or more —CH₂— can be replaced by —O— or —CO—,
Y² represents a C1-C36 aliphatic hydrocarbon group which can have one or more substituents, a C3-C36 saturated cyclic hydrocarbon group which can have one or more substituents, or a C6-C36 aromatic hydrocarbon group which can have one or more substituents, and one or more —CH₂— in the aliphatic hydrocarbon group, the saturated cyclic hydrocarbon group and the aromatic hydrocarbon group can be replaced by —O— or —CO—.

7. A process for producing a photoresist pattern comprising the following steps (1) to (5):
(1) a step of applying the photoresist composition according to claim 1 on a substrate,
(2) a step of forming a photoresist film by conducting drying,
(3) a step of exposing the photoresist film to radiation,
(4) a step of baking the exposed photoresist film, and
(5) a step of developing the baked photoresist film with an alkaline developer, thereby forming a photoresist pattern.
